(19) United States
(12) Patent Application Publication
(10) Pub. No.: US 2025/0001217 A1
(43) Pub. Date: Jan. 2, 2025

(54) SYSTEMS AND METHODS FOR IMAGING AND MODULATING THE NERVOUS SYSTEM USING AN ULTRASOUND-BASED BRAIN-COMPUTER INTERFACE

(71) Applicant: Forest Neurotech, LLC, Cambridge, MA (US)

(72) Inventors: Sumner Lee NORMAN, Los Angeles, CA (US); Tyson Nikiyas Sunyata AFLALO, Claremont, CA (US); William BIEDERMAN, Menlo Park, CA (US); Gianmarco PINTON, Chapel Hill, NC (US); Claire NASTASKIN, Los Angeles, CA (US); Sean FRICK, San Francisco, CA (US); Charles GUAN, Emeryville, CA (US); Phil Joseph GRISILLO, Danville, CA (US)

(21) Appl. No.: 18/759,817

(22) Filed: Jun. 29, 2024

Related U.S. Application Data
(60) Provisional application No. 63/511,617, filed on Jun. 30, 2023.
(60) Provisional application No. 63/598,886, filed on Nov. 14, 2023.

(51) Int. Cl. A61N 7/00

(52) U.S. Cl. CPC A61N 7/00; A61N 2007/0026; A61N 2007/0047; A61N 2007/0052; A61N 2007/0078

(57) ABSTRACT

Devices, methods, and systems related to ultrasound imaging or modulating of the nervous system are described. The devices may comprise, for example, one or more ultrasound transducers, wherein an ultrasound transducer from the one or more ultrasound transducers can comprise an implantable ultrasound transducer, wherein the implantable ultrasound transducer can comprise a sonolucent window. The methods and systems may also comprise, for example, a method of imaging and/or modulating the nervous system of a subject using the one or more ultrasound transducers. The method of imaging and/or modulating the nervous system of the subject can be based on a closed-loop operation, wherein an iteration of the imaging and/or the modulating of the nervous system is based on a prior iteration. The closed-loop operation can comprise ultrasound imaging and ultrasound-based modulating or electrophysiological modulating. The methods can further comprise methods of analyzing ultrasound data, such as via an artificial neural network.

### CROSS-REFERENCE TO RELATED APPLICATIONS

This application claims the priority benefits of U.S. Provisional Patent Application Ser. No. 63/511,617, filed Jun. 30, 2023, and U.S. Provisional Patent Application Ser. No. 63/598,886, filed Nov. 14, 2023, the contents of which are incorporated herein by reference in their entirety.

### FIELD OF THE DISCLOSURE

The present disclosure relates generally to systems and methods for imaging and modulating a physiology, e.g., a nervous system, of a subject, and more specifically to, systems and methods for using implantable ultrasound transducers to image and modulate the nervous system of a subject.

### BACKGROUND

Debilitating brain disorders and diseases that are resistant to treatment or drugs are prevalent. Existing neurotechnology solutions fall short of tackling the complex and individualized nature of human brain dysfunction. Existing solutions can be highly invasive, limited in spatial or temporal resolution, limited in spatial or temporal scope, or can be physically cumbersome to the extent that orthologous measurements of the subject are not readily procured. Advanced monitoring and therapeutic tools are needed to address the limitations of currently available drugs and neurotechnologies.

For instance, neuropsychiatric and cognitive disorders, including depression and neuropathic pain, share common traits. Such disorders occur within circuits and systems distributed spatially throughout the nervous system. As another example, brain states associated with disorders evolve slowly over time, ranging from hours to months. Furthermore, the brain states can vary between people, even across persons diagnosed with identical brain dysfunctions. The distributed and time-evolving nature of the disorders can benefit from a broadscale and long-term approach to imaging and modulating the nervous system, for example, for monitoring or treating pathological brain function.

### BRIEF SUMMARY

The methods and systems discussed herein address a technological issue: the lack of suitable systems and methods for monitoring and manipulating neural activity at sufficiently broad temporal and spatial scales and resolutions for human subjects. Existing systems and methods struggle with addressing this issue, because of fundamental physical and neurophysiological constraints inherent to the technology. The methods and systems disclosed herein comprise an ultrasound-based technology that can monitor and manipulate neural activity in humans at mesoscale or macroscale coverage, including, but not limited to, whole-brain level scales. The methods and systems disclosed herein leverage ultrasound-based physics for achieving macroscale-level interfacing with the brain. The macroscale-level brain computer interface described herein can observe and modulate neural circuit dynamics at scales as broad as brain-wide levels. The methods and systems herein comprise ultrasound-based neurotechnology platforms, which can further comprise a digital diagnostic and therapeutic ecosystem that can support the ultrasound-based platform. The systems and methods disclosed herein can make use of mesoscopic or macroscopic access across brain areas, such as, but not limited to, whole brain access, to achieve improved treatments for brain dysfunctions.

In addition to improving the sensitivity and resolutions over existing methods, functional ultrasound imaging, as described herein, can be packaged into an implantable form factor, unlike, for example, functional magnetic resonance imaging (fMRI). In doing so, the described ultrasound imaging systems can promote high-resolution neuroimaging while subjects are engaged in natural and clinically relevant behaviors. In addition to clinically relevant neuroimaging applications, the disclosed systems can also accomplish neurostimulation of dysfunctional brain circuits, while the subject is engaged in clinically relevant behaviors. The neurostimulation of the subject can be in a closed loop (as discussed further below), such that when a relevant neural activity pattern and/or clinically relevant behavior is observed, therapeutic stimulation of a relevant brain circuit can be achieved. The device packaging described for the systems disclosed herein can also be repurposed for more general applications outside of the interfacing with neural activity, such as for the monitoring and stimulation of non-brain physiological systems.

The systems and methods disclosed herein are overall compact and minimally invasive. The systems and methods comprise at least one, but usually multiple, small ultrasound transducers herein referred to as “implantable transducers,” “implantable sensors,” or “pucks.” The puck is designed to fit in a craniotomy (e.g., a drill hole of diameter 30 mm or smaller) in the skull, which enhances the puck's longevity and minimizes infection risk. The hardware ecosystem supporting the puck can comprise implantable technology, such as, but not limited to, rechargeable batteries and wireless data streaming. In such an implementation, the deployment of the puck for functional ultrasound in the brain can be rapid and relatively inexpensive. In other implementations, the hardware ecosystem controlling the pucks can comprise tailored solutions, such as a custom controller that employs a specialized management scheme for coordinating the constellation of pucks. The software ecosystem supporting the disclosed systems for monitoring and manipulating the brain can comprise tools that provide analyses, visualization, and quantification of relevant metrics and neurological biomarkers. The systems and methods disclosed herein allow for the monitoring and manipulation of neural activity at improved spatial and temporal scales and resolutions, even while the subject engages in clinically relevant behaviors.

The systems and methods for using ultrasound to image and/or modulate the nervous system, as described herein, can be operated in a closed loop, such that the nervous system of a subject is modulated based on the ultrasound-imaged activity of the nervous system. Modulating the nervous system based on the imaging can be performed iteratively, such that the iterated imaging and modulating directs the subject's neural activity towards a target neural activity state, e.g., brain state. The target neural activity state can be a normative neural state, e.g., a neural activity state that does not correspond with a neural state observed or indicative of a subject with a psychiatric pathology.

As an example, neural activity in a region of a subject's brain can be observed with ultrasound data, such as ultrasound imaging using an implantable transducer. The ultrasound data can then be analyzed, such as by a trained machine learning model, so that a set of modulation parameters (e.g., instructions for modulating the subject's neural activity) can be determined. The model can determine the modulation parameters, such that when performing the neuromodulation in accordance with the determined parameters, the observed region would achieve the target neural activity state. The modulation parameters may be communicated to a system for performing the neuromodulations.

The neuromodulation effects can then be observed, for example, via ultrasound imaging, at selected brain regions. The differences between the neural activity (observed via ultrasound imaging) resulting from the modulation and the target neural activity state may be analyzed by a trained machine learning model. The difference can be used to determine updated modulation parameters, such that subsequent neuromodulation, in accordance with the new parameters, can cause the subject's neural activity to converge toward the target neural activity state. When the difference between the observed activity and the target neural activity are smaller than a threshold (e.g., indicating that the observed activity sufficiently reached the target neural activity), the modulation parameters may no longer be updated.

The alternating sequence of observing the subject's neural activity followed by updating of the subject's neural activity modulation can be done in real time. In some embodiments, the alternating sequence of observing and modulating continues for a period of time based on, for example, clinical and biomedical limitations. In some aspects, each iteration of observing and analyzing the subject's neural activity and modulating the subject's neural activity based on the observations, may result in the observed neural activity being closer to the target neural activity, allowing the subject to be more efficiently and accurately treated in a minimally invasive manner and in a more individually tailored manner.

Although examples of methods and systems for imaging and modulating the nervous system are described with respect to the brain, it should be appreciated that the methods and systems may be performed on other parts of the nervous system. The methods and systems can be used on other parts of the nervous system, such as non-brain parts of the central nervous system (e.g., the spinal cord) or the peripheral nervous system. Although examples of methods and systems for imaging and modulating the nervous system are described with respect to ultrasound images, it should be appreciated that the methods and systems may be performed using other kinds of ultrasound data, such as radiofrequency (RF) data.

In some embodiments, the method can further comprise: emitting, via the one or more implantable transducers, ultrasound waves, wherein the ultrasound waves are configured to modify the subject's physiological activity. In any of the embodiments herein, the system can comprise between one and ten implantable transducers.

In some aspects, disclosed herein is an implantable transducer comprising: a housing; a sonolucent window disposed, at least in part, at a first end of the housing; and an ultrasound array disposed within the housing proximate the first end, the ultrasound array configured to emit ultrasound waves to an outside environment via the sonolucent window.

In some embodiments, the implantable transducer can further comprise one or more one circuit boards disposed within the housing, the one or more circuit boards comprising one or more electronic components disposed thereon, the one or more electronic components and configured to send one or more signals to the ultrasound array.

In some embodiments, the one or more electronic components disposed on the circuit board are configured to process data received from the ultrasound array, the data indicative of brain function in a subject.

In any of the embodiments herein, the data comprises image data indicative of anatomical features of a subject.

In any of the embodiments herein, the implantable transducer is configured to be disposed in a hole in a skull of a subject.

In any of the embodiments herein, the implantable transducer is positioned in contact with a soft tissue of a subject.

In any of the embodiments herein, the implantable transducer is configured to cause an increase in local body temperature of less than 2° C.

In any of the embodiments herein, the implantable transducer is configured to limit absolute local brain temperature to less than 39° C.

In any of the embodiments herein, the implantable transducer is positioned in contact with a dura mater of the subject.

In any of the embodiments herein, the implantable transducer is located outside a brain parenchyma of the subject.

In any of the embodiments herein, the housing comprises a lip disposed at a second end of the housing, the lip configured to be mounted to an outer surface of a skull of a subject.

In any of the embodiments herein, the implantable transducer comprises a cable configured to transmit power or data to or from the implantable transducer.

In any of the embodiments herein, the cable protrudes through the housing of the implantable transducer.

In any of the embodiments herein, the sonolucent window comprises a biocompatible polymer.

In some embodiments, the biocompatible polymer is polymethyl methacrylate (PMMA), or Poly(ether) ether ketone (PEEK), polychlorotrifluoroethylene (PCTFE), polytetrafluoroethylene (PTFE), ultra-high-molecular-weight polyethylene (UHMWPE), polyethylene terephthalate (PET), low density polyethylene (LDPE), polyether block amide (PEBAX), and/or high-density polyethylene (HDPE).

In any of the embodiments herein, the ultrasound array is fabricated on a complementary metal-oxide semiconductor (CMOS) application specific integrated circuit (ASIC).

In any of the embodiments herein, the ultrasound array comprises a capacitive micromachined ultrasonic transducer (CMUT), piezoelectric micromachined ultrasonic transducer (PMUT) array or a Lead Zirconate Titanate (PZT) array.

In any of the embodiments herein, the implantable transducer is configured to couple to one or more wires, the implantable transducer configured to send and further configured to receive data via the one or more wires.

In any of the embodiments herein, the implantable transducer is configured to receive a plurality of ultrasound waves.

In any of the embodiments herein, the ultrasound array comprises a plurality of transducer elements.

In some embodiments, the plurality of transducer elements comprises 100-199, 200-399, 400-999, 1,000-1,499, 1,500-9,999, 10,000-11,999, 12,000-99,000, or 100,000-120,000 transducer elements.

In some embodiments, the ultrasound array comprises an n×m matrix, wherein n is in a range of 16-256 transducer elements and m is in a range of 1-256 transducer elements.

In some aspects, disclosed herein is a system for monitoring or modulating a physiological activity of the subject, comprising: one or more implantable transducers, an implantable transducer of the one or more implantable transducers corresponding to the implantable transducer of any of the embodiments herein; and a controller coupled to each of the one or more implantable transducers, the controller comprising a power source and a processor, wherein the power source is configured to power each of the one or more implantable transducers, and wherein the processor is configured to execute a method, the method comprising: sending one or more signals to the one or more implantable transducers; and receiving data from the one or more implantable transducers.

In some embodiments, the system can further comprise: emitting, via the one or more implantable transducers, ultrasound waves, wherein the ultrasound waves are configured to modify the physiological activity of the subject.

In any of the embodiments herein, the one or more signals are configured to specify the amplitude or the timing of one or more transducer elements of the plurality of transducer elements.

In any of the embodiments herein, the system can further comprising between one and ten implantable transducers.

In any of the embodiments herein, the system can further comprise a remote hub, the remote hub configured to receive the data from the controller and further configured to transmit external data to the controller.

In some embodiments, the remote hub can be configured to communicate with a display to provide a user interface for controlling the system.

In any of the embodiments herein, the implantable transducer and the controller are configured to communicate wirelessly.

In some embodiments, the implantable transducer and the controller are configured to communicate via Bluetooth, Bluetooth low energy, WiFi, or a combination thereof.

In any of the embodiments herein, the one or more signals are configured to coordinate emission of ultrasound waves via the implantable transducer and further configured to coordinate receipt of the ultrasound waves.

In any of the embodiments herein, the controller comprises a clock, and wherein the one or more signals are sent based on predetermined intervals associated with the clock.

In some embodiments, the controller comprises a central clock and the one or more implantable transducers each comprise the clock, and wherein the signals correspond to reset signals associated with the central clock.

In any of the embodiments herein, the subject engages in a clinically relevant behavior while the implantable transducer obtains data.

In some embodiments, the clinically relevant behavior comprises activities of daily living, estimates of movement, motion capture, facial expression and response time, self-reported mood, self-reported cognitive state, heart rate, heart rate variability, breathing rate, oxygenation, galvanic skin response, inertial monitoring, or a combination thereof.

In any of the embodiments herein, the system is configured to modify the physiological activity of the subject based on the data.

In some embodiments, modifying the physiological activity of the subject based on the data occurs in real-time.

In some embodiments, the real-time occurrence comprises a response time of 5 seconds or less, after receiving the data.

In some embodiments, modifying the physiological activity of the subject occurs at regular pre-determined intervals.

In any of the embodiments herein, the physiological activity of the subject is neural activity.

In some embodiments, the subject's neural activity is neural activity of the central nervous system.

In some embodiments, the subject's neural activity is neural activity of the brain.

In some embodiments, the neural activity of the brain comprises neural activity from a distributed neural network of the brain.

In any of the embodiments herein, the subject has, or is suspected of having, a neural dysfunction.

In some embodiments, the neural dysfunction is clinical depression, clinical anxiety, neuropathic pain, or a combination thereof.

In some aspects, disclosed herein is a method for monitoring a physiological activity of a subject, the method comprising: sending, via a controller, one or more signals to one or more implantable transducers, wherein the controller is located remotely from the one or more implantable transducers and wherein the one or more implantable transducers are mounted to the skull of the subject; and receiving data from the one or more implantable transducers.

In some embodiments, the method further comprises emitting, via the one or more implantable transducers, ultrasound waves based on the one or more signals, wherein the ultrasound waves are configured to modify the physiological activity of the subject.

In any of the embodiments herein, the method further comprises modifying a physiological activity of the subject based on the ultrasound waves.

In some aspects, disclosed herein is a method for determining instructions for modulating neural activity of a nervous system of a subject, comprising: receiving, from an implantable transducer, ultrasound data of the nervous system, wherein the ultrasound data indicate physiological state of the nervous system; processing the ultrasound data of the nervous system; and transmitting the processed ultrasound data, wherein the instructions for modulating neural activity of the nervous system are determined based on the processed ultrasound data.

In some embodiments, a region of the neural activity being modulated is determined based on the ultrasound data.

In any of the embodiments herein, the method can further comprise receiving one or more of data associated with the physiological state and data associated with the neural activity.

In any of the embodiments herein, the physiological state comprises neurophysiological state.

In some embodiments, the neurophysiological state comprises hemodynamic activity.

In some embodiments, the hemodynamic activity is indicated by power Doppler intensity associated with the ultrasound data.

In any of the embodiments herein, the hemodynamic activity comprises cerebral blood volume (CBV) activity, and changes in the CBV activity are proportional to changes in the power Doppler intensity.

In any of the embodiments herein, the modulating the neural activity comprises stimulating one or more regions of the nervous system.

In some embodiments, the one or more regions of the nervous system comprise one or more regions of a peripheral nervous system, one or more regions of a central nervous system, or a combination thereof.

In some embodiments, the one or more regions of the central nervous system comprise a brain.

In any of the embodiments herein: the stimulating the one or more regions of the nervous system comprises electrical stimulation via one or more electrodes and the instructions for the modulating the neural activity comprise instructions for controlling the electrical stimulation via the one or more electrodes.

In some embodiments, the electrical stimulation is controlled via electrical modulation parameters comprising amplitude, frequency, pulse width, intensity, waveform, polarity, acoustic pressure, or any combination thereof.

In any of the embodiments herein, the electrical stimulation comprises deep brain stimulation (DBS), transcranial magnetic stimulation (TMS), repetitive TMS (rTMS), vagus nerve stimulation (VNS), transcranial direct current stimulation (tDCS), electrocorticography (ECoG), or any combination thereof.

In any of the embodiments herein, the modulating the neural activity comprises ultrasound neuromodulation, and the method further comprises: receiving, by the implantable transducer, the instructions for the modulating the neural activity; and performing, by the implantable transducer, the ultrasound neuromodulation.

In any of the embodiments herein, the modulating the neural activity comprises ultrasound neuromodulation, and the method further comprises: receiving, by the implantable transducer, the ultrasound neuromodulation.

In any of the embodiments herein, the method is performed over a period of seconds, minutes, hours, days, weeks, months, or years.

In any of the embodiments herein, the instructions for the modulating the neural activity are associated with a longitudinal treatment or a longitudinal study.

In any of the embodiments herein, the instructions for the modulating the neural activity are determined further based on pre-trial physiological state information.

In some embodiments, the pre-trial physiological state information comprises pre-trial ultrasound information, functional magnetic resonance imaging (fMRI) information, electrophysiological recordings, structural magnetic resonance imaging scans, diffusion tensor imaging (DTI) information, computed tomography (CT) scan information, or any combination thereof.

In any of the embodiments herein, the instructions for the modulating the neural activity are determined based on an output of a machine learning algorithm.

In some embodiments, the output of the machine learning algorithm is based on the ultrasound data provided to the machine learning algorithm.

In any of the embodiments herein, the machine learning algorithm is trained via pre-trial physiological state information.

In some embodiments, the pre-trial physiological state information comprises ultrasound information, functional magnetic resonance imaging (fMRI) information, electrophysiological recordings, structural magnetic resonance imaging scans, diffusion tensor imaging (DTI) information, computed tomography (CT) scan information, or any combination thereof.

In any of the embodiments herein, the machine learning algorithm comprises reinforcement learning, Bayesian optimization, a generalized linear model, a support vector machine, a deep neural network, or any combination thereof.

In any of the embodiments herein, the machine learning algorithm is trained offline, tested offline, validated offline, or any combination thereof.

In any of the embodiments herein, the ultrasound data comprise radiofrequency (RF) data or in-phase and quadrature (IQ) data.

In any of the embodiments herein, the ultrasound data comprise one or more ultrasound images.

In any of the embodiments herein, the one or more ultrasound images comprise a two-dimensional image, a three-dimensional image, or any combination thereof.

In some embodiments, a resolution of the one or more ultrasound images is 100 microns to 4 mm.

In any of the embodiments herein, an imaging volume of the one or more ultrasound images comprises a spherical sector having a cone radius.

In any of the embodiments herein, the one or more ultrasound images is received at 10 Hz-257 kHz.

In any of the embodiments herein, the instructions for the modulating the neural activity are determined based on a target neural activity.

In some embodiments, the target neural activity is determined via ultrasound imaging, fMRI imaging, electrophysiological recordings, structural magnetic resonance imaging scans, diffusion tensor imaging (DTI), or any combination thereof.

In some embodiments, the target neural activity is determined via the ultrasound data.

In any of the embodiments herein, the target neural activity is determined based on an output of a transfer learning algorithm.

In any of the embodiments herein, the target neural activity is expressed as a composite time-independent state.

In any of the embodiments herein, the target neural activity is expressed as a multi-dimensional timeseries data.

In some embodiments, the multi-dimensional timeseries data comprise temporal resolution or spatial resolution equal to or less than those of the ultrasound data.

In any of the embodiments herein, the method can further comprise: receiving, from the implantable transducer, second ultrasound data of the nervous system, wherein the second ultrasound data indicate a second physiological state of the nervous system;
processing the second ultrasound data; and transmitting the second processed ultrasound data, wherein second instructions for modulating second neural activity of the nervous system are determined based on the second processed ultrasound data.

In some embodiments, the second instructions for modulating the second neural activity comprise adjusted first instructions for the modulating the first neural activity.

In some embodiments, the adjusting the first instructions for the modulating the first neural activity comprises adjusting electrical modulation parameters, spatial modulation parameters, temporal modulation parameters, or any combination thereof.

In some embodiments, the electrical modulation parameters comprise amplitude, frequency, pulse width, intensity, waveform, polarity, acoustic pressure, or any combination thereof.

In any of the embodiments herein, the spatial modulation parameters comprise electrode configuration, electrode position, electrode size, electrode placement, directionality, coil orientation, coil position, stimulation focality, stimulation bilaterality, montage, focus size, target location, or any combination thereof.

In any of the embodiments herein, the temporal modulation parameters comprise bursting, cycling, ramping, frequency, pulse duration, train duration, inter-train interval, total number of pulses, stimulation patterning, duration, inter-stimulus interval, session frequency, pulse repetition frequency, duty cycle, or any combination thereof.

In some embodiments, the methods can further comprise iterating the receiving step, the processing step, and the transmitting step, wherein: instructions for modulating a respective neural activity of the nervous system are determined based on the respective ultrasound data, and
the method ceases after a predetermined number of iterations.

In any of the embodiments herein, the methods can further comprise iterating the receiving step, the processing step, and the transmitting step, wherein: instructions for modulating a respective neural activity of the nervous system are determined based on the respective ultrasound data, and
the method ceases after a predetermined number of iterations.

In some embodiments, the method ceases in accordance with a determination that the subject exhibits a target neural activity for at least a predetermined duration.

In any of the embodiments herein, the method can, in response to the modulating the neural activity, receive, from the implantable transducer, second ultrasound data of the nervous system.

In any of the embodiments herein, the method can further comprise associating the second ultrasound data of the nervous system with the modulated neural activity.

In any of the embodiments herein, the second ultrasound data of the nervous system are received a predetermined period of time after the modulating the neural activity.

In any of the embodiments herein: the neural activity is modulated at a first region of the nervous system, and in response to the modulating the first region of the nervous system, second instructions for modulating second neural activity of the nervous system at a second region of the nervous system are determined.

In any of the embodiments herein, the second instructions are determined a predetermined period of time after the modulating the neural activity.

In any of the embodiments herein, the second instructions are performed a second predetermined period of time after the determining the second instructions.

In any of the embodiments herein, the determining the instructions for the modulating the neural activity comprises determining a region of the nervous system of the modulation based on the ultrasound data.

In any of the embodiments herein, the instructions for the modulating the region of the neural activity are further based on a second physiological state.

In some embodiments, the second physiological state is received with the first physiological state of the nervous system.

In any of the embodiments herein, the second physiological state comprises a behavior of the subject, ocular measurements of the subject, hematological measurements of the subject, or any combination thereof.

In some embodiments, the behavior of the subject is determined based on the subject's response to a questionnaire, a mood assessment, or both.

In any of the embodiments herein, the ocular measurements of the subject comprise eye-tracking or pupil dilation measurements.

In any of the embodiments herein, the hematological measurements of the subject comprise blood pressure, blood glucose levels, blood cholesterol levels, blood hormone levels, or any combination thereof.

In any of the embodiments herein, the second physiological state is determined via a camera, a microphone, a wearable device, or any combination thereof.

In any of the embodiments herein, the wearables device comprises an electronic watch, an electronic ring, or an electronic glasses.

In any of the embodiments herein, the second physiological state is associated with a positive or a negative valence.

In some embodiments, the positive or the negative valence is determined based on pre-trial physiological state observation, the ultrasound data, or both.

In any of the embodiments herein, the positive or the negative valence is determined via experiment.

In any of the embodiments herein, the positive or the negative valence is used, in part, to determine a target neural activity.

In any of the embodiments herein, the modulating the neural activity is associated with treating chronic pain, depression and anxiety, compulsion disorder, Parkinson's Disease, essential tremor, epilepsy, post-traumatic stress disorder, a memory disorder or any combination thereof.

In some embodiments, the compulsion disorder is obsessive compulsive disorder, substance abuse disorder, or both.

In any of the embodiments herein, the subject is a human.

In any of the embodiments herein, the instructions for the modulating the neural activity are transmitted to a neuromodulation system via an interfacing device.

In any of the embodiments herein, the instructions for the modulating the neural activity are transmitted to a neuromodulation system via a communications protocol.

In some embodiments, the communication protocol can comprise USB.

In some aspects, disclosed herein is a method for determining instructions for modulating neural activity of a nervous system of a subject, comprising: receiving, from an implantable transducer, ultrasound data of the nervous system, wherein the ultrasound data indicate physiological state of the nervous system; processing the ultrasound data of the nervous system; transmitting the processed ultrasound data, wherein the instructions for modulating neural activity of the nervous system are determined based on the processed ultrasound data; and receiving, by the implantable transducer, the instructions for the modulating the neural activity; and performing, by the implantable transducer, the ultrasound neuromodulation, on the subject.

In some aspects, disclosed herein is a sonolucent window adjacent to an ultrasound array, comprising: a biocompatible polymer; and configured to permit the transmitting of ultrasound waves through the sonolucent window.

In some embodiments, the biocompatible polymer comprises polymethyl methacrylate (PMMA), polyether ether ketone (PEEK), polychlorotrifluoroethylene (PCTFE), polytetrafluoroethylene (PTFE), ultra-high-molecular-weight polyethylene (UHMWPE), polyethylene terephthalate (PET), low density polyethylene (LDPE), polyether block amide (PEBAX), and/or high-density polyethylene (HDPE).

In any of the embodiments herein, the biocompatible polymer comprises a density greater than or equal to a lower density and less than or equal to a higher density.

In some embodiments, the lower density is approximately 0.31 g/cm3.

In any of the embodiments herein, the higher density is approximately 2.75 g/cm3.

In any of the embodiments herein, the biocompatible polymer is configured to permit the transmission of ultrasound waves at a speed greater than or equal to a predetermined lower speed of sound and less than or equal to a predetermined higher speed of sound.

In some embodiments, the predetermined lower speed of sound is approximately 896 meters per second.

In some embodiments, the predetermined higher speed of sound is approximately 3680 meter per second.

In any of the embodiments herein, the biocompatible polymer comprises an attenuation coefficient greater than or equal to a predetermined lower attenuation coefficient and less than or equal to a higher attenuation coefficient.

In some embodiments, the predetermined lower attenuation coefficient is approximately 0.15 dB/cm/MHz.

In some embodiments, the predetermined higher attenuation coefficient is approximately 9.27 dB/cm/MHz.

In any of the embodiments herein, the biocompatible polymer comprises an impedance greater than or equal to a lower impedance and less than or equal to a higher impedance.

In some embodiments, the lower impedance is approximately 0.685 MRayls.

In some embodiments, the higher impedance is approximately 2.765 MRayls.

In any of the embodiments herein, the biocompatible polymer comprises an impedance ratio greater than or equal to a predetermined lower impedance ratio and less than or equal to a predetermined higher impedance ratio.

In some embodiments, the predetermined lower impedance ratio is approximately 0.625.

In some embodiments, the predetermined higher impedance ratio is approximately 2.765.

In any of the embodiments herein, the biocompatible polymer comprises a reflection coefficient greater than or equal to a lower reflection coefficient and less than or equal to a higher reflection coefficient.

In some embodiments, the lower reflection coefficient is approximately 0.005.

In some embodiments, the higher reflection coefficient is approximately 0.215.

In any of the embodiments herein, the biocompatible polymer comprises a transmission coefficient greater than or equal to a lower transmission coefficient and less than or equal to a higher transmission coefficient.

In some embodiments, the lower transmission coefficient is approximately 0.785.

In some embodiments, the higher transmission coefficient is approximately 0.995.

In any of the embodiments herein, the biocompatible polymer comprises a total attenuation at a predetermined frequency greater than or equal to a predetermined lower total attenuation at the predetermined frequency and less than or equal to a predetermined higher total attenuation at the predetermined frequency.

In some embodiments, the predetermined lower total attenuation is approximately 0.01 dB/cm.

In some embodiments, the predetermined higher total attenuation is approximately 40.95 dB/cm.

In any of the embodiments herein, the predetermined frequency is approximately 5 MHz.

In any of the embodiments herein, the proximity of the enclosed ultrasound array to the sonolucent window comprises the enclosed ultrasound array being adjacent to the sonolucent window.

In any of the embodiments herein, an implantable transducer comprises the sonolucent window, the ultrasound array, and a housing.

In any of the embodiments herein, the implantable transducer comprises a cable configured to transmit power or data to or from the implantable transducer.

In some aspects, disclosed herein is a method of assembling an implantable transducer, comprising: placing an ultrasound array proximate a sonolucent window; and joining a housing to the placed ultrasound array, wherein the placed ultrasound array is disposed, at least in part, in the housing.

In some embodiments, the housing comprises one or more housing components.

In any of the embodiments herein, the joining the housing to the placed ultrasound array comprises joining the one or more of housing components to the placed ultrasound array.

In any of the embodiments herein, the assembling or the joining comprises using a bonding method.

In some embodiments, the bonding method comprises laser welding, electron beam welding, TIG welding, thermal welding, epoxy sealing, or a combination thereof.

In some aspects, disclosed herein is a method of assembling an implantable transducer, comprising: casting a unibody sonolucent housing, wherein the unibody sonolucent housing comprises a sonolucent window and an ultrasound array, and the ultrasound array is disposed within the casted unibody.

In any of the embodiments herein, the sonolucent housing or the sonolucent window comprises a biocompatible polymer.

In some embodiments, the biocompatible polymer comprises polymethyl methacrylate (PMMA), polyether ether ketone (PEEK), polychlorotrifluoroethylene (PCTFE), polytetrafluoroethylene (PTFE), ultra-high-molecular-weight polyethylene (UHMWPE), polyethylene terephthalate (PET), low density polyethylene (LDPE), polyether block amide (PEBAX), high density polyethylene (HDPE), or any combination thereof.

In any of the embodiments herein, the housing comprises a sonolucent material that is not the sonolucent window.

In any of the embodiments herein, the housing comprises a non-sonolucent material.

In any of the embodiments herein, the assembling comprises assembling the implantable transducer in a dry gas environment.

In any of the embodiments herein, the assembling comprises sterilizing the implantable transducer.

In some embodiments, the sterilizing comprises gamma irradiation, autoclaving, ethylene oxide treatment, and/or a combination thereof.

In some aspects, disclosed herein is a method of training a machine learning model comprising: receiving one or more ultrasound data from one or more samples from one or more subjects, obtained from an implantable transducer, and one or more functional ultrasound image data corresponding to the one or more ultrasound data; converting the one or more ultrasound data into one or more ultrasound arrays; converting the one or more functional ultrasound image data into one or more functional ultrasound arrays; and training the machine learning model with the one or more ultrasound arrays and the one or more functional ultrasound arrays, to predict one or more inferred functional ultrasound arrays from inputted one or more ultrasound data or inputted one or more ultrasound arrays.

In some embodiments, the machine learning model is retrained for one or more training iterations, based on additional one or more ultrasound data, additional one or more ultrasound arrays, additional one or more functional ultrasound image data, or additional one or more functional ultrasound arrays.

In some embodiments, the retraining of the machine learning model comprises finetuning the machine learning model based on the additional one or more ultrasound data, the additional one or more ultrasound arrays, the additional one or more functional ultrasound image data, or the additional one or more functional ultrasound arrays.

In any of the embodiments herein, the training the machine learning model further comprises determining one or more metrics that describe a relationship between the one or more inferred functional ultrasound arrays and behavioral data of the one or more subjects.

In any of the embodiments herein, the one or more metrics comprises a correlation metric, a regression metric, a classification metric, a model performance metric, an information theory metric, a temporal metric, or a cross-validated metric.

In some embodiments, the correlation metric comprises a Pearson correlation coefficient, a Spearman's rank correlation coefficient, or a canonical correlation coefficient (CCA).

In some embodiments, the regression metric comprises an R-squared metric, an adjusted R-squared metric, a t-statistic from a generalized linear model (GLM), or an f-statistic from a GLM.

In any of the embodiments herein, the classification metric comprises a decoding accuracy metric, an accuracy metric, a precision metric, a recall metric, an F1 score metric, an area under the receiver operating characteristic curve (AUC-ROC) metric, an area under the precision-recall curve (AUC-PR) metric, or a confusion matrix metric.

In some embodiments, the model performance metric comprises a mean squared error (MSE) metric, a root mean squared error (RMSE) metric, a mean absolute error (MAE) metric, an explained variance metric, or a log-loss metric.

In some embodiments, the information theory metric comprises a mutual information metric or a transfer entropy metric.

In some embodiments, the temporal metric comprises a temporal signal-to-noise ratio (tSNR) metric or a latency of detection metric.

In any of the embodiments herein, the cross-validated metric comprises the metric wherein the metric is cross-validated.

In any of the embodiments herein, the training the machine learning model comprises jointly optimizing the metric and an error between the one or more inferred functional ultrasound arrays and the one or more functional ultrasound arrays.

In any of the embodiments herein, the one or more metrics is used as a part of a cost function, during the training of the machine learning model.

In some embodiments, the cost function includes a weighted sum of the one or more metrics.

In some embodiments, the weighted sum of the one or more metrics are dynamically adjusted during the training of the machine learning model.

In any of the embodiments herein, the behavioral data comprises movement data, cognitive task performance data, emotional state data, or any combination thereof.

In some embodiments, the movement data is obtained from accelerometers, gyroscopes, or motion capture systems.

In some embodiments, the cognitive task performance data is based on reaction times, error rates, or task completion times.

In some embodiments, the emotional state data is obtained from physiological signals such as heart rate, galvanic skin response, surveys, or facial expressions.

In any of the embodiments herein, the training the machine learning model comprises using a regularization technique.

In some embodiments, the regularization technique includes dropout, L1 regularization, or L2 regularization.

In any of the embodiments herein, the training the machine learning model comprises a human-in-the-loop technique.

In some embodiments, the human-in-the-loop technique comprises the assessing of the inferred functional ultrasound array by a medical professional.

In any of the embodiments herein, the machine learning model comprises an attention mechanism.

In any of the embodiments herein, the ultrasound data or the functional ultrasound image data are subject to image enhancing.

In some embodiments, the image enhancing comprises deconvolving or applying super-resolution techniques.

In any of the embodiments herein, the ultrasound data or the functional ultrasound image data for the one or more subjects are paired to clinical metadata corresponding to the one or more subjects.

In some embodiments, the clinical metadata comprises the age, gender, or medical history, of the subject.

In some aspects, disclosed herein is a method of inferring a functional ultrasound array from one or more ultrasound data, comprising: receiving the one or more ultrasound data from one or more samples from one or more subjects; converting the one or more ultrasound data into one or more ultrasound arrays providing the one or more ultrasound arrays to a trained machine learning model; and outputting one or more inferred functional ultrasound arrays, based on the received one or more ultrasound data.

In some embodiments, the method can further comprise converting the one or more inferred functional ultrasound arrays into one or more inferred functional ultrasound image data.

In any of the embodiments herein, the one or more functional ultrasound arrays comprise a power Doppler image.

In any of the embodiments herein, the one or more ultrasound data comprise a sequence of ultrasound data.

In any of the embodiments herein, the ultrasound data comprise radiofrequency data, in-phase and quadrature (IQ) data, B-mode image data, compound image data, or any combination thereof.

In any of the embodiments herein, the ultrasound data comprise radiofrequency data, in-phase and quadrature (IQ) data, compound image data, or any combination thereof, and not B-mode image data.

In any of the embodiments herein, the trained machine learning model is trained on training data comprising the ultrasound data and functional ultrasound image data.

In any of the embodiments herein, at least a portion of the training data comprise normalized image data or augmented image data.

In some embodiments, the normalized image data comprise color-normalized image data.

In some embodiments, the augmented image data comprise image data that have been augmented by removing noise from the image data, improving contrast of the image data, adjusting brightness of the image data, performing convolution against an image kernel and/or geometric transformation.

In some embodiments, convolution against an image kernel comprises convolving against a Gaussian blurring kernel, a box blurring kernel, an edge detection kernel, a sharpening kernel, an unsharp masking kernel, or any combination thereof.

In some embodiments, the geometric transformation comprises affine transformation, elastic transformation, flipping, cropping, grid distortion, optical distortion, perspective transformation, transposition, or any combination thereof.

In some embodiments, the affine transformation comprises translation, rotation, scaling, shearing, or any combination thereof.

In any of the embodiments herein, the training data is split into a first training data fraction, a first test data fraction, and a validation data fraction.

In some embodiments, the first training data fraction comprises 70%, 75%, 80%, 85%, or 90% of the training data, the first test data fraction comprises 20%, 18%, 15%, 13%, 10%, or 5% of the training data, and the validation data fraction comprises 20%, 18%, 15%, 13%, 10%, or 5% of the training data.

In any of the embodiments herein, the validation data fraction comprises one or more training image patches, and the first training data fraction comprises all training images excluding the one or more training images in the validation data fraction.

In any of the embodiments herein, the training data is split into a second training data fraction, and a second test data fraction.

In some embodiments, the second training data fraction comprises 60%, 65%, 70%, 75%, or 80% of the training data and the second test data fraction comprises 40%, 35%, 30%, 25%, or 20% of the training data.

In any of the embodiments herein, the training data is subject to a cross-validation.

In some embodiments, the cross-validation comprises k-fold cross-validation, leave-p-out cross-validation, leave-one-out cross-validation, stratified k-fold cross-validation, repeated k-fold cross-validation, nested k-fold cross-validation, or Monte Carlo cross-validation.

In any of the embodiments herein, the machine learning model comprises an encoder-decoder architecture.

In any of the embodiments herein, the machine learning model comprises a 3-D convolutional filter.

In some embodiments, the 3-D convolutional filter is configured to extract one or more spatiotemporal features from the one or more ultrasound data, the one or more ultrasound arrays, the one or more functional ultrasound image data, or the one or more functional ultrasound arrays.

In any of the embodiments herein, the machine learning model comprises residual blocks.

In any of the embodiments herein, the machine learning model comprises a convolutional neural network (CNN).

In some embodiments, the CNN comprises a convolution function, an activation function, a pooling function, or any combination thereof.

In some embodiments, the convolution function comprises convolving a matrix from the input against a kernel.

In some embodiments, the kernel is initialized randomly and learned from training the neural network.

In some embodiments, the learning comprises backpropagating and optimizing.

In some embodiments, the optimizing comprises gradient descent, stochastic gradient descent, batch gradient descent, mini-batch gradient descent, Adam optimization, AdaGrad optimization, RMSprop optimization, momentum optimization, or any combination thereof.

In any of the embodiments herein, the activation function is a rectified linear unit (ReLU) function, a leaky ReLU function, a linear activation function, a non-linear activation function, a sigmoid activation function, or a hyperbolic tangent activation function.

In any of the embodiments herein, the pooling function is a max pooling function, an average pooling function, or an attention-based pooling function.

In any of the embodiments herein, the machine learning model further comprises a softmax function or an argmax function.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

The patent or application file contains at least one drawing executed in color. Copies of this patent or patent application publication with color drawings will be provided by the Office upon request and payment of the necessary fee.

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a schematic representing an example system that can be used for ultrasound-based imaging or modulating of physiological activity, according to some embodiments.

FIG. 2A provides an example non-implanted configuration of a peripheral controller and transducer devices (e.g., ultrasound transducers) that can deliver ultrasound pulses, according to some embodiments.

FIG. 2B provides an example implanted configuration of a peripheral controller and transducer devices (e.g., ultrasound transducers) that can deliver ultrasound pulses, according to some embodiments.

FIGS. 3A and 3B provides examples of an implantable transducer that can deliver ultrasound pulses, according to some embodiments.

FIGS. 4A-4C provide external views of an example implantable transducer that can deliver ultrasound pulses, according to some embodiments.

FIG. 5 provides a schematic illustrating an example circuit of an ultrasound transducer that can deliver ultrasound pulses, according to some embodiments.

FIGS. 6A and 6B provide renderings of an example ultrasound transducer device, according to some embodiments.

FIG. 7 provides example ultrasound transducer devices, according to some embodiments.

FIG. 8A provides a cutaway rendering of an example ultrasound transducer device, according to some embodiments.

FIG. 8B provides a rendering of an exterior of an example ultrasound transducer device, according to some embodiments.

FIG. 8C provides a line drawing of an exterior of an example ultrasound transducer device, according to some embodiments.

FIG. 8D provides a line drawing of an exterior of an example ultrasound transducer device, according to some embodiments.

FIG. 8E provides a rendering of an exterior of an example ultrasound transducer device.

FIGS. 9A and 9B provide exploded view renderings of an example ultrasound transducer device, according to some embodiments.

FIGS. 10A and 10B provide renderings of an example ultrasound transducer device, according to some embodiments.

FIG. 11 depicts an exemplary workflow for assembling an ultrasound transducer device, according to some embodiments.

FIG. 12 provides a schematic depicting an ultrasound transducer device connected to a personal computer, according to some embodiments.

FIG. 13A provides an example peripheral controller that can control one or more ultrasound transducer devices that can deliver ultrasound pulses, according to some embodiments.

FIG. 13B provides an example peripheral controller that can control one or more non-implantable transducers that can deliver ultrasound pulses, according to some embodiments.

FIG. 13C provides an example peripheral controller that can control one or more implantable transducers that can deliver ultrasound pulses, according to some embodiments.

FIGS. 14A and 14B provide example peripheral controllers connected to multiple transducers that can deliver ultrasound pulses, according to some embodiments.

FIG. 15A provides a diagram illustrating an example use of an implantable transducer, according to some embodiments.

FIG. 15B provides a diagram illustrating an example implementation of multiple implantable transducers in the skull of a subject, according to some embodiments.

FIG. 15C provides example ultrasound transmit regimes for imaging and neuromodulation applications, according to some embodiments.

FIG. 15D provides a diagram illustrating an example use of multiple implantable transducers, according to some embodiments.

FIG. 16A depicts an exemplary workflow for iteratively modulating the nervous system based on observed neural activity, according to some embodiments.

FIG. 16B depicts an exemplary workflow for iteratively modulating the nervous system with ultrasound neuromodulation, based on observed neural activity, according to some embodiments.

FIGS. 17A and 17B provide schematics representing example neural activity states, according to some embodiments.

FIG. 18 provides schematics representing example neural activity states following neuromodulating in accordance with modulation parameters, according to some embodiments.

FIG. 19 depicts an exemplary schematic depicting adjustment of neuromodulation parameters to achieve desired brain state.

FIG. 20 depicts an exemplary workflow for iteratively modulating the brain based on observed neural activity, according to some embodiments.

FIG. 21 depicts an exemplary workflow for iteratively modulating the brain based on observed neural activity, according to some embodiments.

FIG. 22 provides an example diagnostic and/or therapeutic platform, according to some embodiments.

FIG. 23A depicts an exemplary method for training a machine learning model for reconstructing one or more functional ultrasound images, according to some embodiments.

FIG. 23B depicts an exemplary method for reconstructing a functional ultrasound image from anatomical ultrasound images, according to some embodiments.

FIG. 24 depicts an example computing device or system, according to some embodiments.

FIG. 25 depicts an example computer system or computer network, according to some embodiments.

FIG. 26 provides an example of data depicting the simulation results, according to some embodiments.

FIGS. 27A-27J provide examples of data associated with the simulation-based imaging design, according to some embodiments.

FIG. 28 provides examples of data depicting the results of a simulation, according to some embodiments.

FIG. 29A provides an example plot of pressures applied to the brain of a subject, when delivering pulsed wave amplitudes with ultrasound transducer devices.

FIG. 29B provides an example plot of materials used for delivering the pulsed wave amplitudes with the ultrasound transducer devices shown in FIG. 29A, such that FIG. 29B can be overlaid onto FIG. 29A.

FIG. 30A provides an example 3D plot of pressures when applying ultrasound from two non-implantable ultrasound transducer devices to a subject, according to some embodiments.

FIG. 30B provides an example 3D plot of pressures when applying ultrasound from three implantable ultrasound transducer devices to a subject, according to some embodiments.

FIGS. 31A-31G provides example data describing the ultrasound imaging quality and transparency of an ultrasound transducer comprising one of several sonolucent materials, according to some embodiments.

FIG. 32 provides example tabular data describing the physical properties of ultrasound waves passing through one of several sonolucent materials, according to some embodiments.

FIG. 33 provides an image of an example ultrasound transducer configured to deliver ultrasound waves, being tested in an experimental chamber, according to some embodiments.

FIGS. 34A-34E provides data describing properties of one or more ultrasound pulse delivered by an ultrasound transducer device in an experimental chamber, according to some embodiments.

FIGS. 35A-35C provide example hydrophone data of one or more ultrasound pulses as a function of space, when delivered by a non-implantable ultrasound transducer device, according to some embodiments.

FIGS. 36A-36C provide example simulation data of one or more ultrasound pulses as a function of space, when delivered by an implantable ultrasound transducer device, according to some embodiments.

FIG. 37A provides an example activity map resulting from ultrasound imaging of a brain, according to some embodiments.

FIG. 37B provides an example neural activity trace, based on the ROI indicated in FIG. 37A.

FIG. 38 provides an image of an example ultrasound transducer applying one or more ultrasound pulses to a subject, according to some embodiments.

FIG. 39A provides a schematic of an example coronal section of the human brain, according to some embodiments.

FIG. 39B provides example imaging data of neural activity from a subject's brain, obtained from ultrasound imaging with an ultrasound transducer, according to some embodiments.

FIGS. 40A and 40B provide example imaging data of neural activity from a subject's brain, obtained from ultrasound imaging with an ultrasound transducer, according to some embodiments.

FIGS. 41A-41E provide example imaging data of neural activity from subjects' brains, obtained from ultrasound imaging with an ultrasound transducer, according to some embodiments.

FIGS. 42A-42B provide example imaging data of neural activity from a subject's brain, obtained from ultrasound imaging with an ultrasound transducer, according to some embodiments.

FIGS. 43A-43D provide example power Doppler imaging data of neural activity from a mouse brain, including reconstructed imaging data of the mouse brain, based on a trained artificial neural network model, according to some embodiments.

### DETAILED DESCRIPTION

Disclosed herein are devices, methods, and systems for imaging and stimulating the brain, using ultrasound. Neurotechnology, based on devices that read from or write to the brain, holds the promise to cure neurological and psychiatric conditions, enhance human experience through improved cognition, memory, and sleep, and enable high bandwidth communication with technology and to each other. Neurotechnologies have not yet, however, delivered on this promise because the current approaches are either incomplete or poorly matched to the biology of the brain. For example, current neurotechnologies often sacrifice temporal and/or spatial scale and resolution. The methods and systems disclosed herein address this technological shortcoming within the field. Namely, the methods and systems disclosed herein provide the ability to monitor and manipulate neural activity at sufficiently broad, but precise, temporal and spatial scales and resolutions for human subjects, even during clinically relevant behaviors without penetrating the brain of a subject.

Existing systems and methods struggle with providing appropriate scales and resolutions for monitoring and manipulating neural activity, because of fundamental physical and neurophysiological constraints inherent to the technology. For example, electrophysiological systems, while providing sufficient temporal resolution in terms of sampling frequencies, often cannot be used for extended recording sessions, because of the technique's surgical invasiveness (e.g., implantation directly in the brain of the subject). The feasibility of monitoring uninterrupted electrophysiological activity across weeks or months often results in extended durations of contact between the probe and the brain of the subject, and can increase the risk of infection. Electrophysiological methods also tend to provide very specific spatial resolutions, but at the expense of coverage. Multiple recording probes may often be implemented to achieve even mesoscale monitoring of neural activity. The invasiveness and poor scaling properties of electrophysiological methods limit the impact of such technologies.

Functional magnetic resonance imaging (fMRI) is another existing technology that is often non-ideal for reading and writing neural activity. Magnetic fields, like those produced by an fMRI machine, can easily penetrate and image deep brain structures, but fMRI machines and fMRI-based technologies are difficult to miniaturize. As a result, fMRI technologies are often unsuitable for making real-time localized interactions with neuronal circuits. In addition, fMRI technologies often fail to record neural activity during clinically relevant behaviors, because the unwieldiness of fMRI machines often require the subject to be immobile. In short, the existing modalities capable of reading and writing neural activity struggle to balance invasiveness with performance.

The methods and systems disclosed herein can bridge the gap between the invasiveness and performance of current neurotechnologies. The methods and systems discussed herein leverage the physical properties of ultrasound. Ultrasound possesses wavelengths of approximately 100 microns, and travels at speeds similar to that of sound, in soft tissue, at approximately 1.5 km/s. These physical properties allow for ultrasound energy to propagate throughout the brain at about 100 microns of spatial resolution and about 1 ms of temporal resolution. Ultrasound technology can be focused deeply within tissues. For example, focused ultrasound (FUS) is a rapidly growing therapeutic method for neuromodulation and tissue ablation. Ultrasound's affordability, portability, and safety make it suitable for use in clinical medicine and facilitate its application for macro-scale brain imaging and modulation. Technology based on ultrasound physics can bridge the gap between invasiveness and performance. The methods and systems described herein relate to a brain-computer interface (BCI) that can image and/or modulate the neural activity of a subject's nervous system, using ultrasound. The imaging and/or modulating of the neural activity can be achieved via implantable ultrasound transducers. The transducers can propagate ultrasound waves, and record the reflected wave patterns of the propagations, to infer the neural activity of the subject and/or modulate the neural activity of the subject. The transducers can be connected to a peripheral controller, which can provide power and/or organize the activities of the multiple transducers (e.g., the constellation of the transducers). The peripheral controller can offload the collected neural activity data to an external server for further processing, which can include analyzing the data based on algorithms, including machine learning algorithms.

BCIs based on developments in ultrasound physics, as described herein, can be implemented in closed-loop methods comprising strategic neuromodulation of a subject, based on ultrasound imaging of the subject. For example, the imaging and the modulating of the nervous system may complement one another, such that a target neural activity state (e.g., for treating a nervous system disorder or disease) is achieved via iterations of imaging and modulating. The iterations of imaging and modulating can both be based on ultrasound physics, such as via implantable ultrasound transducers. In some examples, the iterations of imaging and modulating can be based on both ultrasound-based and electrophysiological methods, such as by using implantable ultrasound transducers for imaging the subject's neural activity and using electrodes for modulating the subject's neural activity.

In addition to providing high-performing and relatively non-invasive techniques of imaging and manipulating neural activity, the systems and methods disclosed herein can also further the development of other fields. For example, advances in the field of molecular biology and gene therapy are paving the way for advances that would be further accelerated by the methods and systems discussed herein. For example, when paired with intravenous microbubbles, ultrasound can temporarily open the blood-brain barrier for the precision delivery of drugs in the brain. Precision delivery can also be achieved by using ultrasound to uncage drugs. Sonogenetic approaches can also be used to leverage the interactions between ultrasound waves and genetically modified cells, to promote targeted manipulation of neural activity. The methods and systems disclosed herein can accelerate the development of these methods, and open new avenues in neurological and personalized medicine.

The systems and methods disclosed herein can also promote the advances in silicon manufacturing. When coupled with state-of-the art low-power integrated circuits, ultrasound-on-chip technologies enable systems and methods for reading and writing neural activity, such as those described herein. The methods and systems described herein synergize with recent advancements with other fields, and can provide macroscale longitudinal recording and modulating of neural activity at highly improved temporal and spatial resolutions.

The section headings used herein are for organizational purposes only and are not to be construed as limiting the subject matter described.

### Definitions

Unless otherwise defined, all of the technical terms used herein have the same meaning as commonly understood by one of ordinary skill in the art in the field to which this disclosure belongs.

As used in this specification and the appended claims, the singular forms “a,” “an,” and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain’), are inclusive or open-ended and do not exclude additional, un-recited additives, components, integers, elements, or method steps.

As used herein, ordinal terms such as “first,” “second,” “third,” etc., in the claims to modify a claim element does not by itself connote any priority, precedence, or order of one claim element over another, or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, “modulating,” in reference to a subject, can refer to the process of altering the activity of the nervous system of the subject. The modulating of the nervous system can be referred to as “neuromodulating.” Neuromodulating with ultrasound can involve the delivery of focused ultrasound waves to specific regions of the nervous system to influence neural activity. Modulation can result in a variety of effects on the subject, such as stimulating or inhibiting neural firing, altering synaptic transmission, modifying the release of neurotransmitters, and influencing intracellular signaling pathways. The modulating may be used to enhance or suppress neural signals, and/or alter how the modulated region may respond to exogenous or endogenous neural signals, which may not comprise the direct exciting or suppressing of the subject's neural activity.

As used herein, “imaging” via ultrasound waves, e.g., ultrasound imaging, can refer to various types of imaging, including non-functional imaging (e.g., anatomical imaging), and/or functional imaging. The ultrasound imaging can comprise qualitative ultrasound imaging or quantitative ultrasound imaging. Non-functional imaging, e.g., anatomical imaging, can be optimized for capturing detailed anatomical structures of the nervous system, without capturing time-varying values of physiological processes, e.g., dynamics. Functional imaging can be optimized for capturing dynamic physiological processes, such as blood flow, and can be used to infer the blood flow of a subject, such as cerebral blood flow, which in turn can be used to infer the neural activity of the subject. Quantitative ultrasound imaging can involve the measurement and analysis of ultrasound wave properties, providing additional information about tissue characteristics and composition. Moreover, “imaging” via ultrasound waves, as used herein, need not refer strictly to the obtaining of images, e.g., visualized data that can be interpreted as mapping onto physiological landmarks via visual correspondences. Imaging via ultrasound waves can refer to the obtaining of any data resulting from the transmitting of ultrasound waves to a subject, which can be visualized downstream via operations to the data. Such data can include, but is not limited to, radiofrequency data and/or its derivatives, or in-phase and quadrature (IQ) data and/or its derivatives.

### Ultrasound-Based Brain-Computer Interface (BCI) System

The methods and systems described herein for monitoring and modulating neural activity can comprise several parts. They can comprise, but are not limited to, at least one implantable transducer (e.g., ultrasound transducer), a peripheral controller for the implantable transducer(s), sonolucent materials for ultrasound-based medical device packaging of the implantable transducer, and methods of analyses related to ultrasound imaging and stimulating, such as, but not limited to, multi-transducer imaging algorithms. Combinations of the aforementioned parts can comprise a system or method relating to a macroscale BCI.

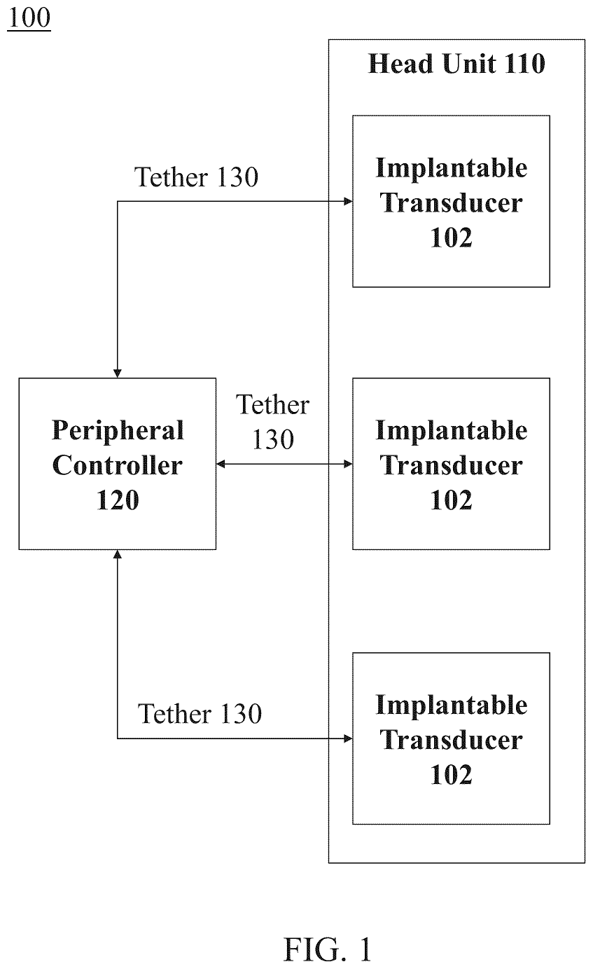

FIG. 1 illustrates an exemplary macroscale BCI system 100. The exemplary system 100 may include at one or more implantable transducers 102, a peripheral controller 120, and one or more tethers 130, the tethers 130 may be configured to be coupled between each implantable transducer 102 and the peripheral controller 120. In some embodiments, the tether 130 can be attached to a rigid body of the implantable transducer 102 and be configured to transmit and receive data from a peripheral controller 120. In some embodiments, the tether 130 may be configured to deliver power from a battery residing in the peripheral controller 120 to the implantable transducer 102. In some embodiments, the one or more implantable transducers can be configured to be mounted on the head of a subject, and may collectively be referred to as a head unit 110. In some embodiments, the peripheral controller 120 is configured to be an implantable unit and configured to be implanted in the chest of the subject for controlling the one or more implantable transducers 102 (e.g., head unit 110). The implantable transducer may comprise a rigid body configured to be mounted to the skull such that the device minimally impacts activities of the subject's living body. The implantable transducer can be configured to detect and/or modulate blood flow or other aspects related to the brain tissue.

In some embodiments, the implantable transducers 102 can be tethered to an external unit for power and data processing, and can function and be compatible with existing technologies, for efficient scaling and implementation. In another embodiment, the macroscale BCI system can comprise a fully integrated system capable of implantation, making the system suitable for chronic free-living clinical research and treatment.

### Implantable Transducers 102

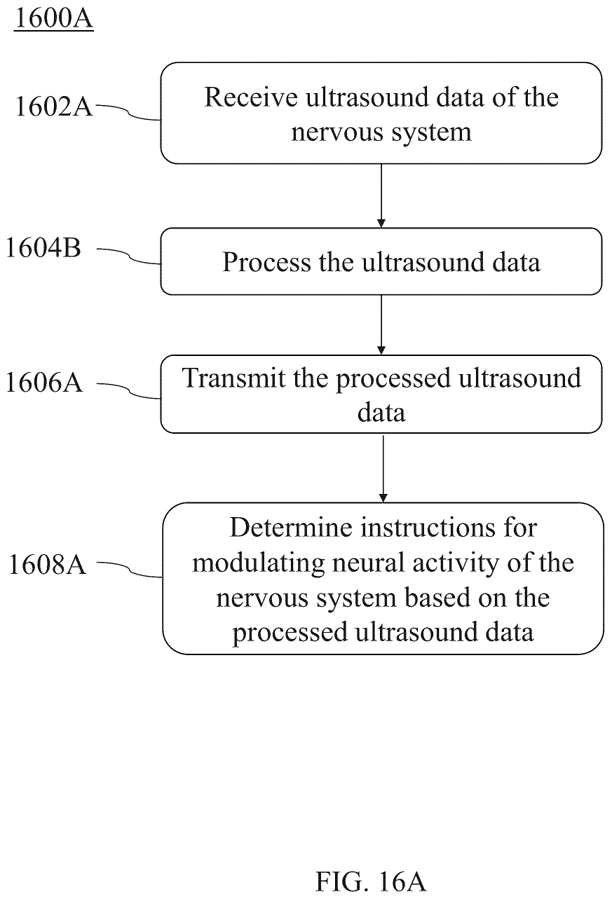

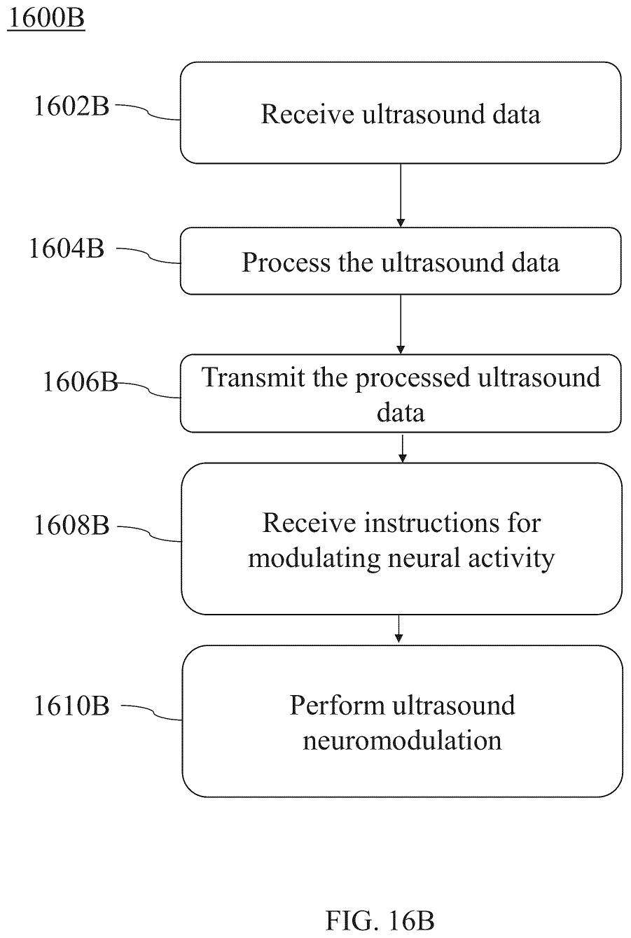

In addition to the broader scale that ultrasound data affords, such as those data that are acquired according to the embodiments described herein, including methods shown in FIGS. 16-21, ultrasound-based imaging allows for greater flexibility when used for multi-modal or multi-dimensional measurements. Ultrasound data can be received using one or more implantable transducers comprising ultrasound arrays.

The implantable transducers 102 may be minimally invasive, in that each transducer may be installed in a small burr hole in the skull of a subject, and may not require excessive penetration into brain tissue. The transducers 102 may be located atop the brain, in line with the skull, such that the physical dimensions of the transducers 102 may not penetrate past the subarachnoid space and into the brain of a subject.

In addition, implantable ultrasound transducers 102 can be highly miniaturized. For example, the total volume of a transducer 102 may be comparable to that of a coin. The convenient form factor of ultrasound transducers 102 allows maintaining a broad and ethological behavioral repertoire. In contrast, traditional methods of observing neural activity such as fMRI and some electrophysiological techniques may be cumbersome and invasive and abrogate a subject's natural behavioral repertoire. For example, fMRI may require that the subject lie relatively still in a small imaging chamber. As a result, the ability to identify neural activity states indicative of a brain disorder may be limited. Baseline behaviors that are known indicators of brain disorders cannot be correlated to ongoing neural activity patterns, because the confines of the fMRI machine may preclude the subject from displaying the known behavioral indicators of the brain disorders. Similarly, many electrophysiological systems for observing neural activity may restrict a subject's natural behaviors. For example, they often require invasive surgical implantations that can require one or more electrodes to pierce into the subject's brain. Some traditional methods, such as stereo electroencephalography (sEEG), are reserved for subjects with intractable forms of epilepsy. Ultrasound-based imaging is less invasive and less restrictive of a subject's natural behavioral repertoire. In addition, the implantable ultrasound transducers 102 can penetrate deep into the brain tissues of a subject, such that less superficial regions of the brain can be more clearly observed. The combination of the broader imaging scale and the flexibility of ultrasound-based imaging provides advantages over traditional technologies for observation-modulation paradigms of neural activity. These advantages also allow the disclosed system to integrate with neurosensing or neuromodulation systems, such as MRI, fMRI, PET, EEG, TMS, or optogenetic tools.

In addition to the implantable transducers' capacity for miniaturization, the implantable transducers 102 can provide a quantity of data that allows for the determining of neuromodulation instructions. For example, multiple aspects of the ultrasound data may allow instructions for neuromodulation to be determined: imaging resolution, image volume, and frequency of capture, as described in more detail herein.

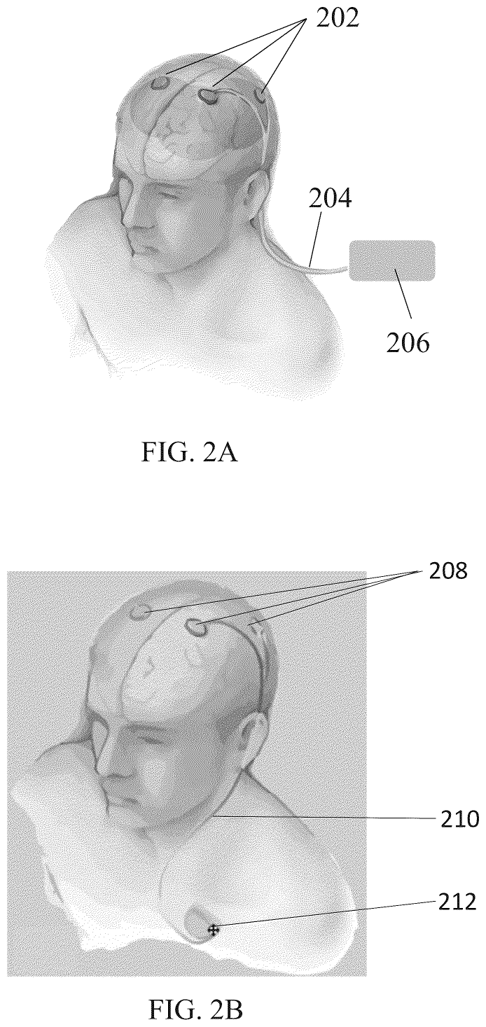

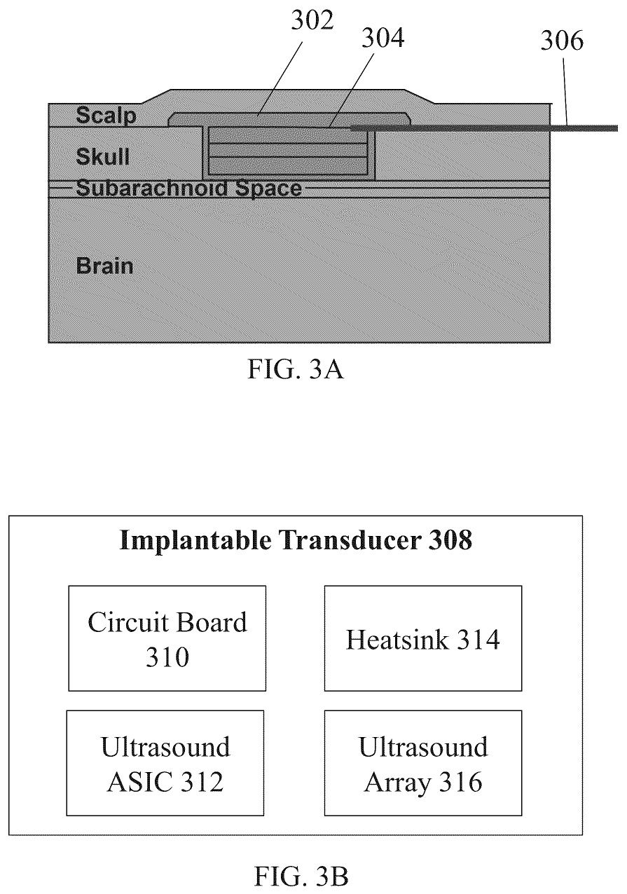

The methods and systems described herein can comprise an ultrasound neural sensing and stimulation device enclosed by a rigid housing designed to mount to the skull of a living human, as shown in FIGS. 2A-3A. FIGS. 2A-2B show two configurations by which ultrasound transducers and a peripheral controller that controls the ultrasound transducers, e.g., a chest unit, can function together to provide a platform for ultrasound-based imaging and modulating. In FIG. 2A, the ultrasound transducers 202 and peripheral controller 206 are not implanted in a way that may permit the free behavior of the subject. For example, the subject may not necessarily be able to behave post-operatively with the ultrasound transducers 202 and peripheral controller 206, given the configuration depicted in FIG. 2A. In FIG. 2A, the cable 204 leads away from the subject, and towards the peripheral controller 206, which is also not implanted in the subject. The cable may be provided to the subject, percutaneously. In accordance with the non-implanted peripheral controller, the ultrasound transducers 202 need not be implanted, but may be provided during a surgery, e.g., for the duration of only the surgery.

In contrast, in FIG. 2B, the ultrasound transducers 208, cable 210, and peripheral controller 212 are implanted into the subject, such that the subject can behave freely post-surgery. The ultrasound transducers 208 can be implantable transducers, given that the ultrasound transducers are implanted, and the peripheral controller 212 can be a chest unit, given that the peripheral controller is implanted, e.g., proximate the chest of the subject. An ultrasound transducer of the ultrasound transducers 208 can be configured to fit within a hole in the skull (e.g., a burr hole), and sit in contact with the dura mater, completely outside the brain parenchyma. The burr hole need not be strictly circular in shape, and can be any shape, such that the burr hole can optimally accommodate for the physical positioning of the ultrasound transducer of the ultrasound transducers 208, e.g., the ultrasound transducer can mate with the burr hole. Accordingly, the form factor of the ultrasound transducer is not intended to limit the scope of this disclosure and other shapes may be contemplated.

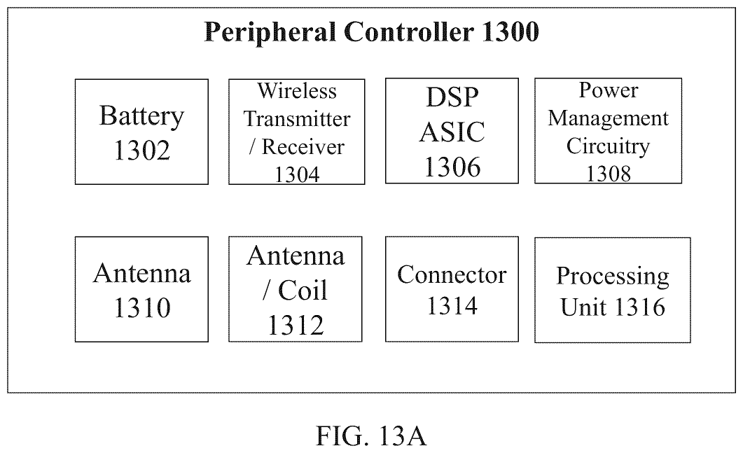

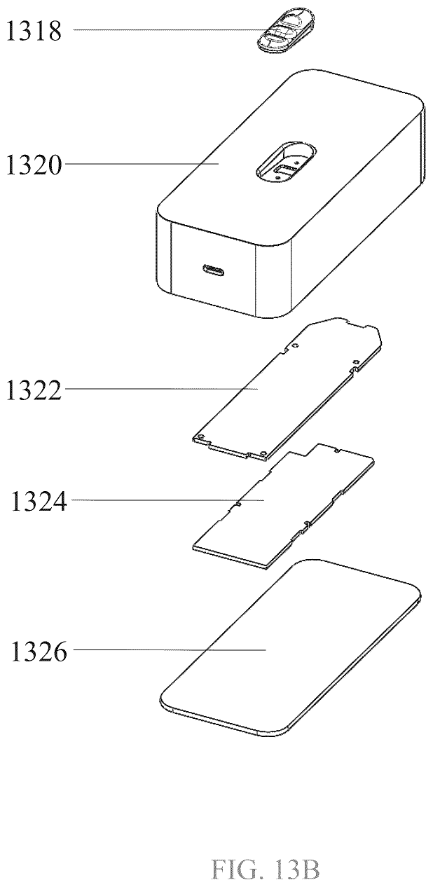

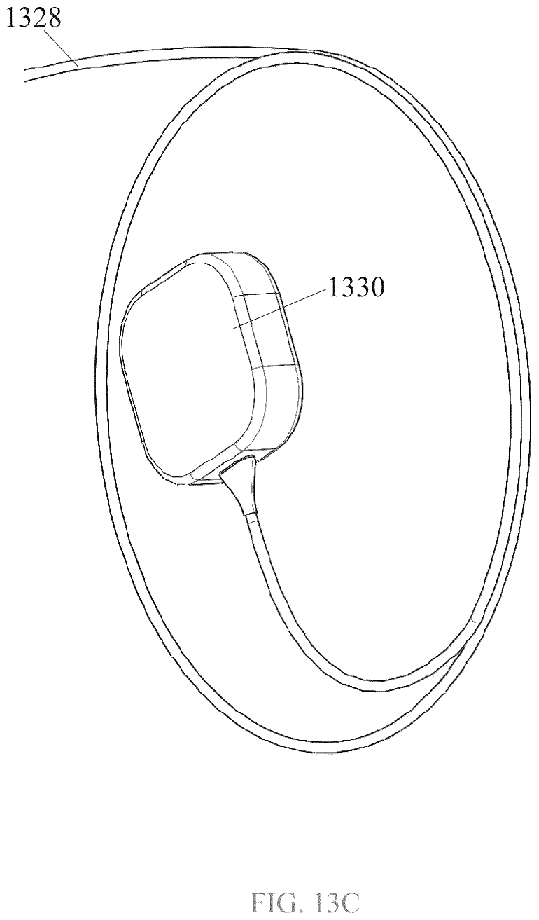

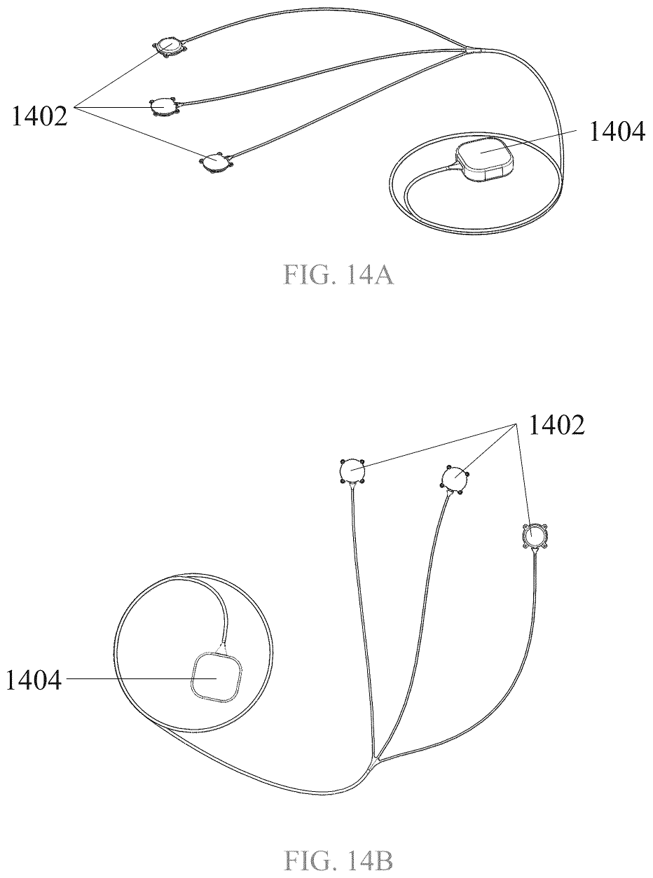

FIGS. 3A and 3B provide examples of an implantable transducer that can deliver ultrasound pulses. In some implementations, as shown in FIG. 3A, the implantable ultrasound transducer 304 can be positioned to be in contact with a soft tissue of a subject, such as, but not limited to, muscles, fat, blood vessels, nerves, tendons, or any combination thereof. Due to its use within the body, the implantable ultrasound transducer 304 can be hermetically sealed and sterilized within a housing 302. The implantable transducer 304 can be connected to a cable 306, which can provide power and/or data to and/or from the implantable transducer 302. The cable 306 can connect the implantable transducer 202 to a peripheral controller, as shown in further detail in FIGS. 13A-13C, and/or other implantable transducers, as shown in FIGS. 14A and 14B.

FIG. 3B illustrates an exemplary schematic of an implantable transducer 312 in accordance with embodiments of this disclosure. As indicated in FIG. 3B, the implantable transducer 308 can comprise a circuit board (PCB) 310, an ultrasound ASIC 312, a heat sink, 314, and a microelectromechanical system (MEMS) ultrasound array 316, all of which can be comprised within a housing, e.g., enclosure, which can include a sonolucent window. The sonolucent window can comprise one or more layers disposed in a housing of the implantable transducer 308. In some embodiments, the mechanical housing may be constructed from biocompatible, chemically stable, and corrosion-resistant materials, e.g., polymers, to withstand the physiological environment imposed by the subject, and/or to possess optimal thermal dissipation properties. In some examples, the housing may be constructed from medical grade stainless steel 316L or titanium. The mechanical shape of the housing can be approximately cylindrical, but the cylindrical form factor is not intended to limit the scope of this disclosure, and other shapes may be contemplated. The implantable transducer 312 can also possess a power source, which can be charged via inductive charging.

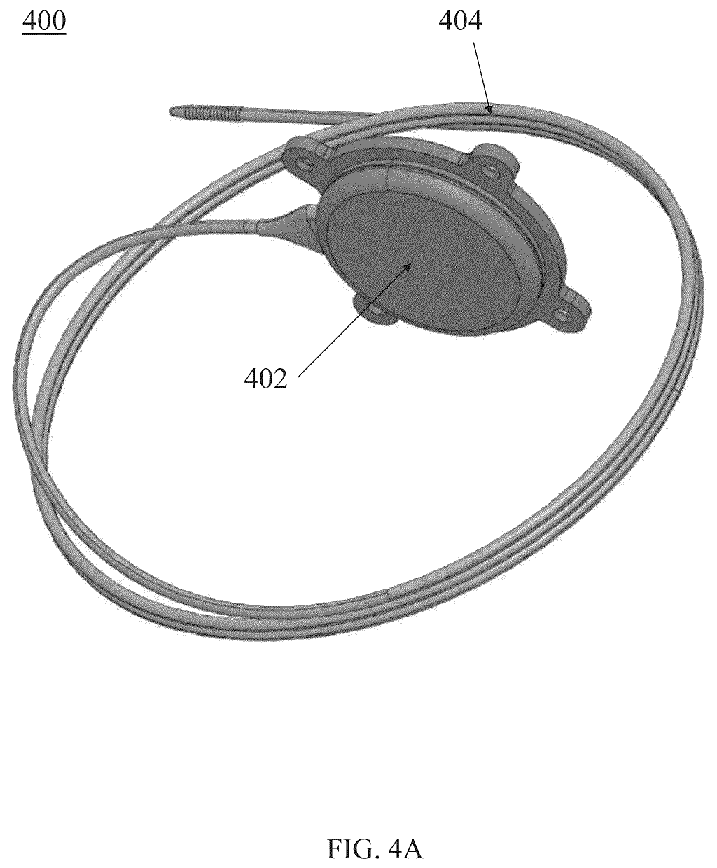

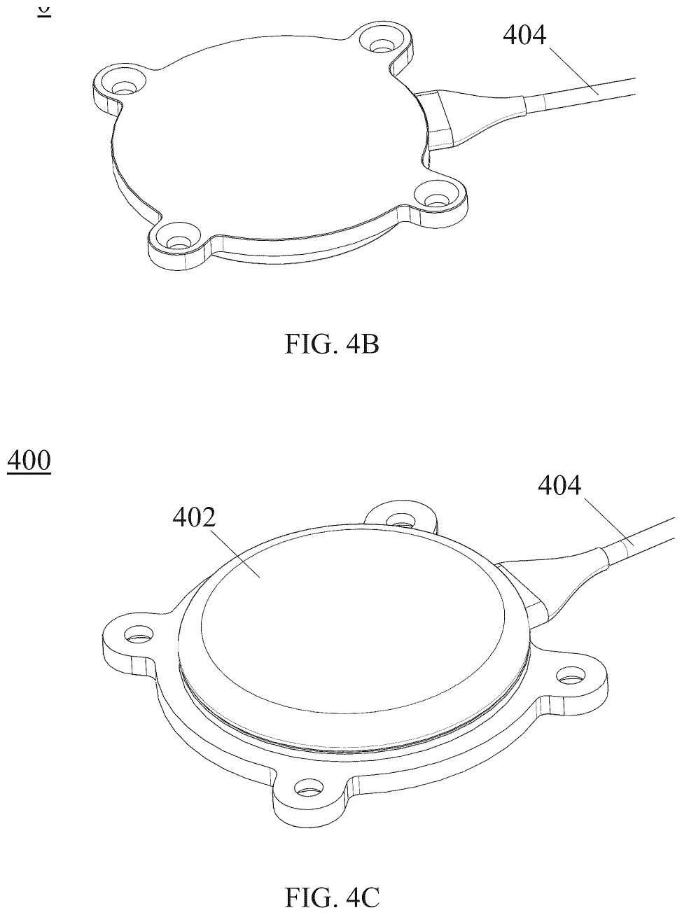

FIGS. 4A-4C provide external views of an implantable ultrasound transducer, which can be in accordance with the implantable ultrasound transducer shown in FIGS. 3A-3B. The implantable ultrasound transducer 400 shown in FIGS. 4A-4C can accommodate a dual-function closed-loop system for both imaging and modulating the nervous system of a subject. The implantable ultrasound transducer 400 shown in FIG. 4A-4C can comprise a cable 404, which can provide bidirectional data and/or unidirectional power from the peripheral controller to an ultrasound transducer 400. The data communication protocol can operate in accordance with a standardized communications protocol, such as a USB protocol, e.g., USB 3 or a subversion of USB 3. Alternatively, the data communication protocol can operate in accordance with a non-standardized, such as proprietary, communications protocol. FIG. 4A-4C also depicts a sonolucent window 402, through which ultrasound waves pass from the ultrasound array (e.g., MEMS ultrasound array) contained inside the housing of the implantable ultrasound transducer 400. The sonolucent window 402 can provide a barrier between the ultrasound array contained in the housing of the implantable ultrasound transducer 400, and the physiological environment, e.g., the subarachnoid space or brain of the subject, such that the ultrasound (e.g., ultrasonic) waves are permitted to transmit through the sonolucent window 402 of the housing without significant attenuation or distortion, while the internal electronics of the ultrasound transducer remain protected. In some embodiments, the sonolucent materials can comprise polymethyl methacrylate (PMMA), polyether ether ketone (PEEK), polychlorotrifluoroethylene (PCTFE), polytetrafluoroethylene (PTFE), ultra-high-molecular-weight polyethylene (UHMWPE), polyethylene terephthalate (PET), low density polyethylene (LDPE), polyether block amide (PEBAX), and/or high density polyethylene (HDPE), or other biocompatible polymers with low acoustic impedance and minimal ultrasound attenuation—that is, sonolucent materials—may be used for the sonolucent window 402.

### Implantable Transducer: Ultrasound ASIC 312

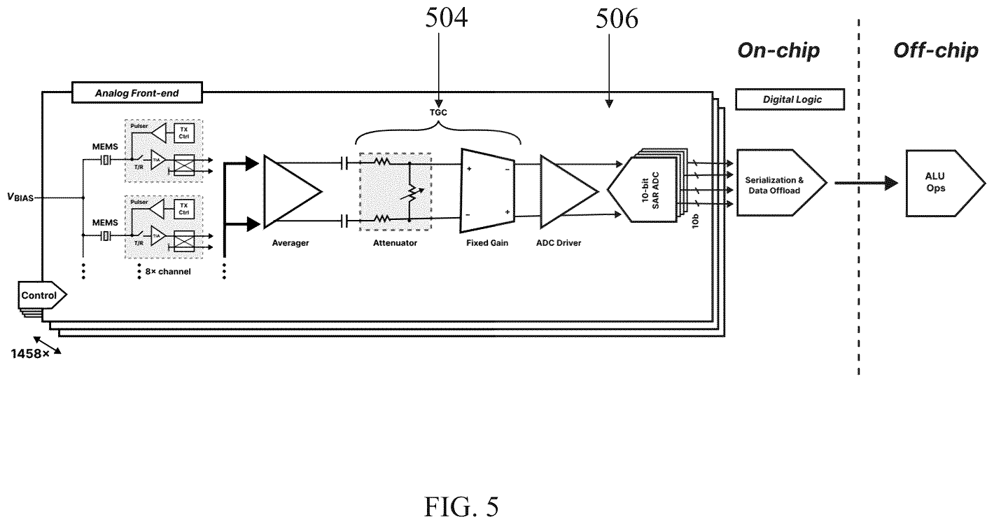

In one or more embodiments, the ultrasound ASIC 312 may comprise one or more analog front end (AFE) circuits, as shown in FIG. 5. In one or more embodiments, at least one AFE circuit can be on a single ASIC. In some embodiments, the AFE circuits may be disposed across two or more ASICs. In some embodiments, the AFE circuits, or a portion thereof, may be distributed across one or more ASICs and/or the circuit board 310. For instance, one or more circuit board components can contribute to AFE functionality. The AFE circuits can be responsible for transmitting ultrasound energy as well as amplifying and digitizing the signals received by the ultrasound ASIC 312.

The AFE circuit may be designed based on one or more factors. For example, power consumption and heating can be a concern because excessive temperature rise can be detrimental to the surrounding tissue (there may be a limit of 2° C. increase allowable to surrounding tissue). The AFE circuit may be designed to minimize power consumption, to reduce the overhead or need for heat dissipation in the head unit 110, and/or to be able to meet the limited battery capacity available in the implantable transducer 304 and/or head unit 110.

Additionally or alternatively, the AFE circuit may be designed based on the amount of data that may be offloaded. In some instances, the disclosed methods and systems can preserve as much of the raw data as possible for scientific discovery, as well as for image processing and algorithm development. Raw data rates from a 10b Nyquist sampling ADC of 10k CMUT (capacitive micromachined ultrasound transducer) elements can produce 1 Tbps of data, for example. This amount of data can be excessive, depending on the intended application, both compared to the fundamental information the disclosed methods and systems attempt to measure as well as the technical challenge to offload and process.

The choice of semiconductor technology for the AFE circuit can directly impact the circuit's overall performance, power consumption, and/or high voltage requirements. In addition, the methods and systems discussed herein can use a MEMS-compatible process on wafers. The wafers can be of any size, such as, but not limited to, approximately 1, 2, 3, 4, 5, 6, 7, 8, 9, or 10 inch wafers. A BCD (Bipolar-CMOS-DMOS) or a Silicon on Insulator (SOI) process can be suitable for manufacturing the AFE circuit, because either the BCD or SOI process can allow for high-voltage devices on a traditional CMOS wafer, while still offering the benefits of a smaller process node. The process can be manufactured in accordance with one or more foundries, and the process can be manufactured in accordance with one of several different options, for the given foundry. For example, a 180 nm BCD 8″ process can meet requirements for high-voltage devices wafer size and overall analog performance.

The system-level architecture of the AFE, shown in FIG. 5, enhances the overall performance and efficiency of the ultrasound imaging system. The system-level architecture of the AFE can be designed to optimally interface with highly capacitive input of the capacitive micromachined ultrasound transducer (CMUT) array and amplify signals with time-varying and beamforming requirements. The AFE can comprise at least the transmitter (TX), the low-noise amplifier (LNA), time-gain control (TGC), and successive approximation register analog-to-digital converter (SAR ADC). These components can be arrayed across transducer elements and work together to form a coherent and efficient signal processing chain for this purpose.

### AFE Circuits: Data Transfer

In some examples, the low-noise amplifier (LNA) can be the first stage of the receiver chain in the AFE, responsible for amplifying the signals received from the CMUT array without introducing significant noise or distortion. For a CMUT ultrasonic transducer, a transimpedance amplifier (TIA) architecture can be appropriate due to its ability to convert the small capacitive current generated by the CMUT elements into a voltage signal, while providing sufficient gain and bandwidth for the application. The TIA input impedance can be designed to match the CMUT array to minimize the input-referred noise.

Following the LNA, the time-gain control (TGC) stage 504 can be used to compensate for the signal attenuation caused by ultrasound waves traveling through different tissue layers. The TGC 504 can apply a time-varying gain to the amplified signals, compensating for distance dependent attenuation of ultrasound signals by boosting the weaker signals that have traveled deeper into the tissue while preserving the overall dynamic range. Applying a time-varying gain to the amplified signals can help ensure that the signals from different depths are appropriately balanced, allowing for a more accurate and detailed reconstruction of the ultrasound image.

Following the TGC stage, an analog-to-digital converter (ADC) 506 can be used to digitize the data for further processing before offloading to an external device. A successive approximation register (SAR) ADC can offer a combination of high resolution, high-speed conversion, and low power consumption, making this form of technology well-suited for ultrasound imaging applications.

Given the imaging approaches disclosed herein, an estimated raw data rate can be a value within a range, such as, but not limited to, approximately 100, 120, 140, 160, 180, 200, 220, 240, 260, 280, 300, 320, 340, 360, 380, 400, 420, 440, 460, 480, 500 Mb/s per functional image. In some embodiments, the ultrasound transducer can use a wireline transceiver to send the data to an external processing unit for scientific research and more advanced algorithm development. In some embodiments, wireless techniques for data transfer may be used.

### Implantable Transducer: AFE Circuit for Signal Transmission

The AFE circuit may further comprise transmitter circuitry for an ultrasonic array in accordance with embodiments of this disclosure. The design of transmitter circuitry for the ultrasonic array can involve a combination of analog components and digital control logic to provide precise beamforming and high-resolution imaging. The basic components of the analog circuitry can include a pulse generator, a high-voltage switch matrix, and a digital-to-analog converter (DAC). The pulse generator can produce high-frequency, short-duration electrical pulses required to excite the CMUT elements. These pulses can be selectively routed to individual elements in the array through a high-voltage switch matrix. The DAC can generate finely-tuned voltage waveforms to control the amplitude and phase of the pulses for each transducer element, thus allowing for precise control of the acoustic pressure fields.

The control logic for beamforming can steer and focus the transmitted ultrasonic wavefront. The control of the transmitted ultrasonic wavefront can be achieved by adjusting the time delays and the amplitude of the excitation pulses applied to each element in the array. The control logic can use a beamforming algorithm, which can incorporate the desired transmit focus and steering angle, along with the geometric configuration of the CMUT array. By calculating the appropriate time delays and amplitudes for each element, the control logic can ensure that the acoustic waves emitted by individual elements constructively interfere at the desired focal point, thereby creating a well-defined, steerable acoustic beam.

In ultrasound, transmit beamforming is a type of signal processing technique used to control the propagation of acoustic waves emitted by multiple transducer elements in an array. The central idea behind transmit beamforming is to adjust the phase and amplitude at each transducer element to create constructive interference at a specific focal point or desired spatio-temporal acoustic profile. The timing delay for each transducer can be calculated based on the intended focus depth and the speed of sound in the tissue. For a wave to constructively interfere at a particular focus point, it should reach that point at the same time from one or more (e.g., all) transducers. Transducers farther from the focus point may be activated slightly earlier than those closer. These delays may be achieved using a delay line in the beamformer circuit.

The process of controlling the timing and amplitude of the signals to achieve, e.g., a focused beam, is not limited to a single point. By changing these parameters dynamically, it is possible to steer the focus point over time. Furthermore, more complex wave shapes can be formed by optimizing these parameters, such as planar waves or diverging waves, for specific imaging tasks.

### Implantable Transducer: MEMS Ultrasound Array

The ultrasound array is structured to optimize performance and precision for both the ultrasound imaging and neuromodulation. The acoustical design of ultrasound imaging arrays is defined in terms of center frequency, total and active channel counts, array shape and geometry, number of arrays, element distribution, bandwidth, and angular sensitivity. Additionally or alternatively, the neuromodulation array designs can be defined in terms of focal volume size and distribution as a function of location within the acoustical envelope, achievable acoustic outputs such as the mechanical index, spatial and temporally averaged intensities and how they vary as a function generate to these outputs using acoustical simulations of the arrays, propagating in both homogenous and heterogenous in silico models of the brain.

As shown in FIG. 3B, the implantable ultrasound transducer can include a MEMs ultrasound array 316 configured to be coupled to custom circuitry (e.g., circuit board 310 and/or ultrasound ASIC 312). The MEMs ultrasound array 316 can be configured to perform imaging of the nervous system, e.g., brain, and can provide neuromodulation to targeted areas for clinical treatment. In one or more embodiments, the MEMs ultrasound array 316 can comprise, for example, a capacitive micromachined ultrasound transducer (CMUT) array or lead zirconate titanate (PZT) transducer array that can be directly fabricated upon, bonded, or otherwise electronically connected, to a complementary metal-oxide semiconductor (CMOS) application-specific integrated circuit (ASIC).

In one or more embodiments, the ultrasound array technology choice can vary, depending on the intended application, to ensure performance, reliability, and scalability. For instance, CMUTs can offer several advantages over traditional piezoelectric transducers, including higher receive sensitivity, broader bandwidth, better integration with electronics, lower acoustic impedance, and reduced crosstalk. These benefits, combined with CMUTs' abilities to be monolithically integrated with CMOS and manufactured at scale, make CMUTs suitable for use in the systems and methods disclosed herein. In some instances, large scale commercial MEMS fabrication facilities can be used to manufacture and/or source the components used for the systems disclosed herein. By leveraging state-of-the-art CMUT technology and integrating it with CMOS, the transducer design can meet the long-term requirements for the head implantable unit.

In one or more examples, the system level specifications for the CMUT array can be tailored to meet the unique imaging requirements in the brain. For instance, a center frequency of 5 MHz, CMUTs can demonstrate bandwidths greater than 100%, which can be tunable from 2.5 MHz to 7.5 MHz, for the methods and systems disclosed herein. These properties can allow for users to selectively configure imaging resolution and depth, depending on application requirements. To achieve a sufficient field of view for macroscale, e.g., near whole-brain imaging and modulation, an aperture of 17 mm with a pitch of 150 microns between elements of the CMUT array can be used, which can result in an element count of 11,660 (imaging/modulation solutions may not require all elements to be used simultaneously). The pitch between elements for the CMUT array, e.g., 150 microns, can be based on the

$$( \frac{λ}{2} )$$

requirement for a maximum coverage angle (e.g., 90 degrees of steerability) and the wavelength of the nominal operational frequency can be 5 MHz. For example, the following calculation can be used to approximate the suitable pitch between elements of the CMUT array: given that a) λ/2, where λ is wavelength; b) v=fλ, where v is the speed of sound, which can be approximately 1540 m/s; c) f is frequency, which in some embodiments, can be approximately 5 MHz; then, given the information in a), b), and c), λ/2 can have a value of approximately 150 μm, which can translate to the pitch between elements of the CMUT array being 150 μm, at a 5 MHz center frequency. In accordance with embodiments of this disclosure, a CMUT array can deliver high-resolution images with broad coverage and optimal performance, while remaining compatible with CMOS manufacturing processes.

The design of the implantable transducer disclosed herein can incorporate various power optimization techniques, such as clock gating and power gating. Such techniques can help to minimize power consumption by selectively turning off or reducing power to parts of the implantable transducer (e.g., circuit board, ASIC, ultrasound array) that are not in use or can operate at a lower performance level.

### Implantable Transducer: Mechanical Assembly

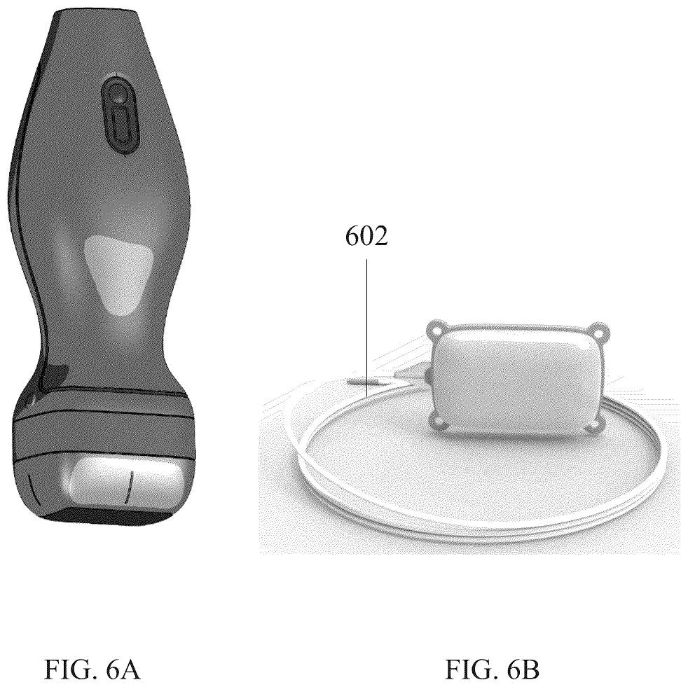

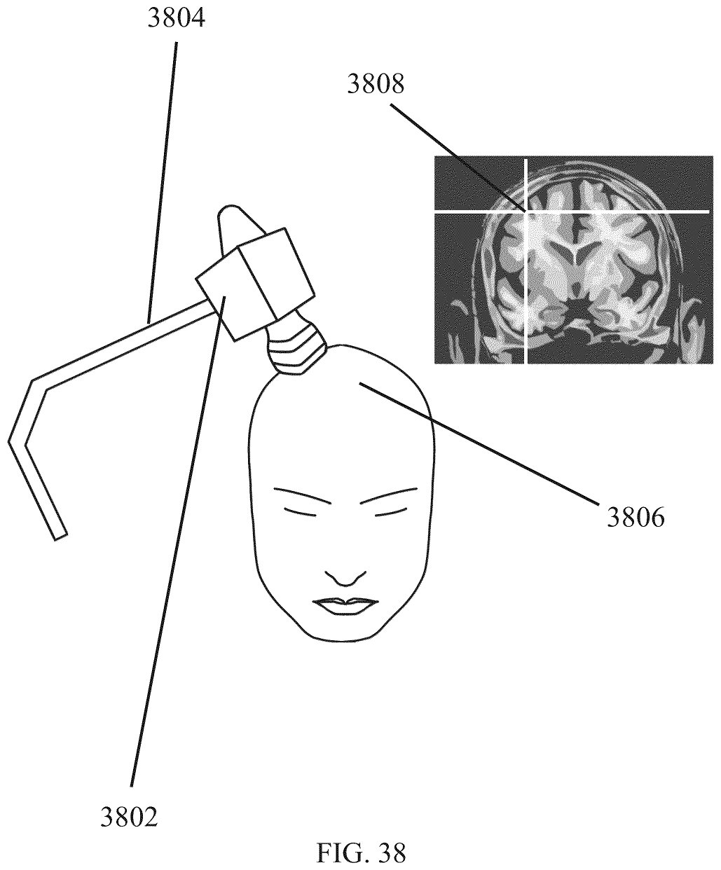

The ultrasound transducer, e.g., the implantable transducer, and its production method can encompass a hermetically sealed housing made from sonolucent materials, such as, but not limited to, polymethyl methacrylate (PMMA), polyether ether ketone (PEEK), polychlorotrifluoroethylene (PCTFE), polytetrafluoroethylene (PTFE), ultra-high-molecular-weight polyethylene (UHMWPE), polyethylene terephthalate (PET), low density polyethylene (LDPE), polyether block amide (PEBAX), and/or high density polyethylene (HDPE). FIGS. 4A-4C depict physical prototypes of an implantable ultrasound transducer. The ultrasound transducer for ultrasound-based BCI can be built in accordance with one or more form factors, as depicted in FIGS. 6A-10B, depending on the application of the ultrasound transducer. For example, FIG. 6A depicts a CAD rendering of a large form factor ultrasound transducer. The large form factor ultrasound transducer may not necessarily be implantable with respect to a subject, but may allow a clinician, e.g., a surgeon, a high degree of manual control over positioning the ultrasound transducer, as depicted in FIG. 38.

In some embodiments, the ultrasound transducer can be an implantable transducer, as shown in FIG. 6B. FIG. 6B depicts a CAD rendering of a small form factor ultrasound transducer. The small form factor ultrasound transducer can be of a form factor that is small enough to permit the transducer to be implanted into the skull of the subject, for which the positioning of the transducer relative to the skull is depicted in FIG. 3A. The ultrasound transducer may operate in accordance with a data transmission protocol, may be connected to a power supply, and may function in accordance with a thermal management that is compatible with cranial implants in humans. The development boards, e.g., printed circuit boards (PCBs) used for the ultrasound transducer, can comprise a multi-part, e.g., two-part, design that can accommodate for an approximate communication rate of, for example, 1, 2, 3, 4, 5, 6, 7, 8, 9, 10, 11, 12, 13, 14, 15, or 16 Gb/s across a cable 602 that can, for example, be an approximate length of 0.5, 0.8, 1.0, 1.5, 2.0, 2.5, 3.0 m.

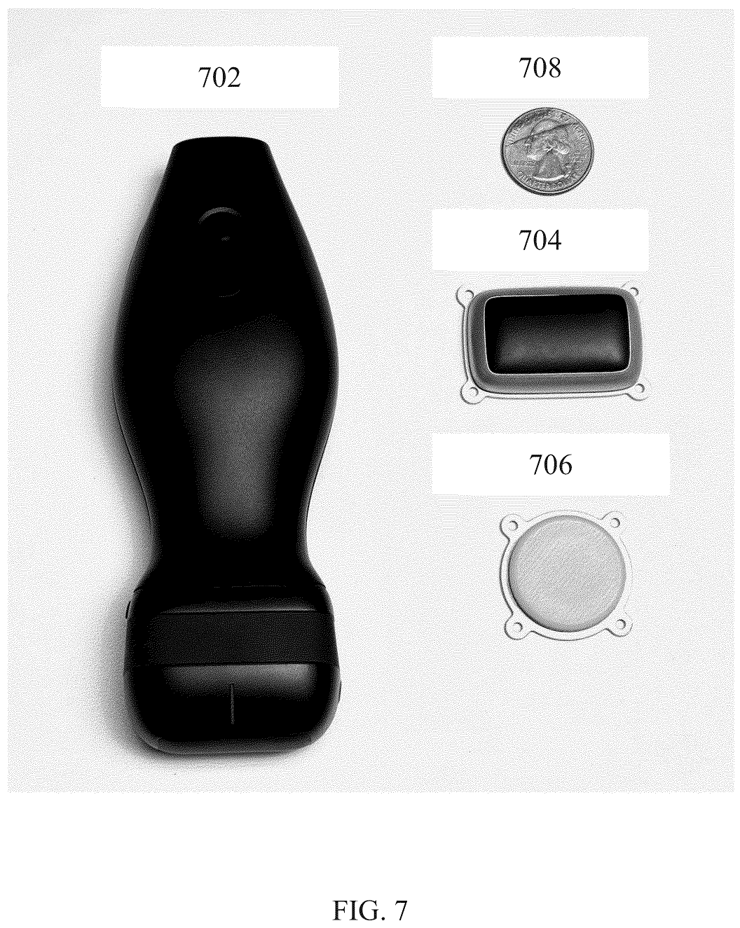

FIG. 7 provides an image depicting various form factors for the ultrasound transducer, such as a large form factor ultrasound transducer 702 (similar to the large form factor ultrasound transducer shown in FIG. 6A), a small form factor implantable ultrasound transducer 704 (similar to the small form factor ultrasound transducer shown in FIG. 6B), and a miniature form factor implantable transducer 706. In some aspects, the miniature form factor implantable transducer 706 can be of a smaller form factor than the small form factor implantable ultrasound transducer. FIG. 7 also depicts a quarter 708, to provide an understanding of scale, relative to the ultrasound transducers 702, 704, and 706.

The implantable transducer can comprise a form factor that is small enough to permit implanting the transducer into the skull of a subject. Accordingly, the form factor of the implantable transducer can be tailored to the cranial geometry of the subject, such that the dimensions of the transducer can range between approximately 3 to 20 mm in thickness and 14 to 50 mm in width and/or length. Optionally, the enclosure may include a cable capable of transmitting both power and data to and/or from the device, ensuring efficient operation. The cable can permit relay of data, e.g., data communication, at an approximate communication rate of, for example, 1, 2, 3, 4, 5, 6, 7, 8, 9, 10, 11, 12, 13, 14, 15, or 16 Gb/s across a cable that can, for example, be an approximate length of 0.5, 0.8, 1.0, 1.5, 2.0, 2.5, 3.0 m

The electronics included within the ultrasound transducer, including any of the implantable transducers depicted in FIGS. 6A-10B, can comprise at least two development boards that can constitute a multi-part (e.g., two part) assembly. More generally, the implantable transducer can be manufactured by securely joining multiple sections of the housing, e.g., in a dry gas environment, to safeguard the internal electronics. Alternatively, the implantable transducer can be manufactured not by constructing a multi-part assembly of the housing, but by constructing a single part, e.g., unibody housing that can directly encapsulate, e.g., hermetically seal, the included electronics, thereby forming a cohesive interface, which can be sterilized. The sterilizing can comprise gamma irradiation, autoclaving, ethylene oxide treatment, and/or a combination thereof. The assembly of the ultrasound transducer, including the forming of the hermetic seal, can be accomplished, at least in part, via a bonding method, such as laser welding, electron beam welding, TIG welding, thermal welding, epoxy sealing, or a combination thereof. The housing can be made of any of the sonolucent materials described above, such as the PMMA, PEEK, PCTFE, PTFE, UHMWPE, PET, PEBAX, LDPE, and/or HDPE material.

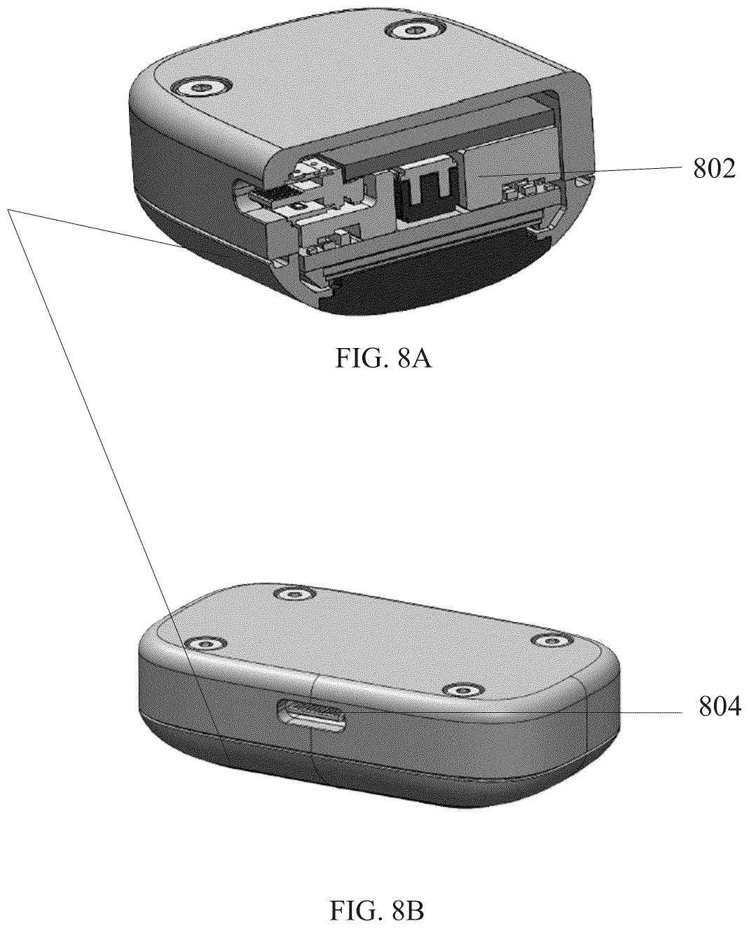

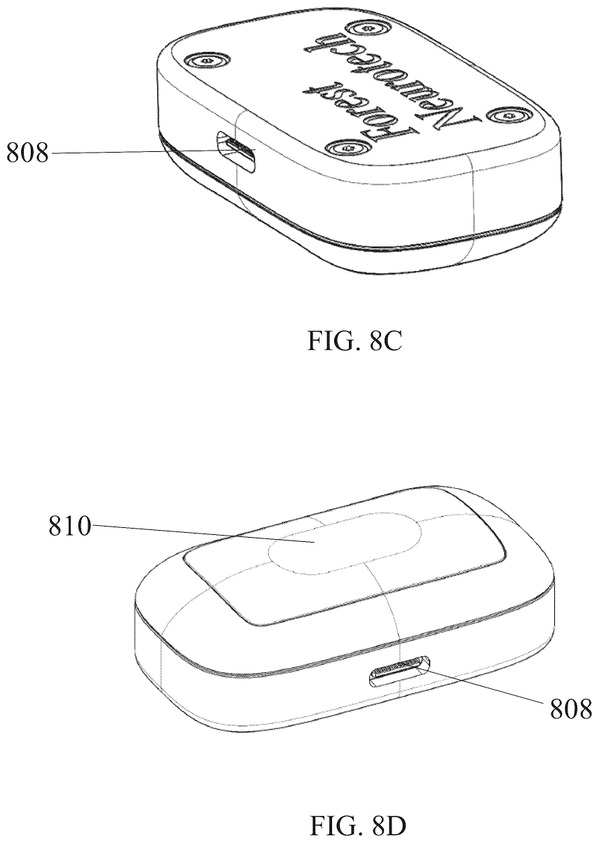

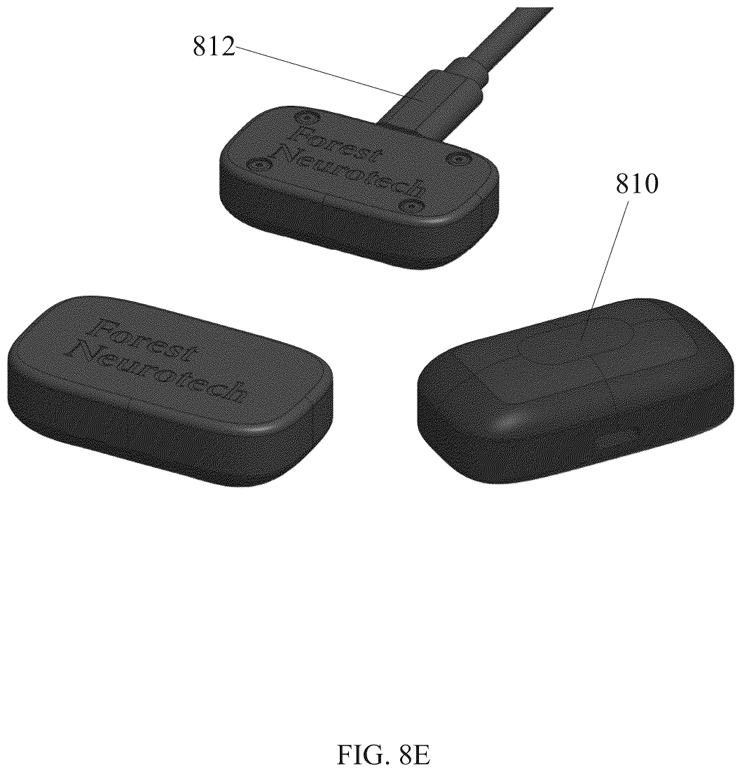

FIGS. 8A and 8B depict additional images demonstrating the assembly of the small form factor ultrasound transducer. FIG. 8A depicts a cutaway rendering of a non-implantable small form factor ultrasound transducer, which reveals the positioning of the inside electronics, including the MEMS ultrasound assembly 802, inside the housing of the ultrasound transducer. FIG. 8B depicts an exterior rendering of the ultrasound transducer, which is of the small form factor depicted in FIG. 7. FIG. 8B also depicts a port 804 for a data cable, such as a USB cable or a proprietary cable that may operate in accordance with a non-standardized data communication protocol. FIGS. 8A and 8B each depict a sonolucent material 806, through which the ultrasound waves pass through. The sonolucent materials can, for example, comprise the PMMA, PEEK, PCTFE, PTFE, UHMWPE, PET, LDPE, PEBAX, and/or HDPE material.

FIGS. 8C and 8D depict line drawings of the exterior of the small form factor non-implantable ultrasound transducer. FIGS. 8C and 8D depict views of the exterior of the small form factor non-implantable ultrasound transducer comprising a port 808 for a cable, which can receive and/or provide bidirectional data and/or unidirectional power from an external device, such as a peripheral controller, as shown in FIGS. 13A-13B, or a computer, to the ultrasound transducer. FIGS. 8A-8E depict a version of the small form factor non-implantable ultrasound transducer that is manufactured according to multiple housing components. FIG. 8D depicts a sonolucent window 810, through which ultrasound waves, e.g., ultrasound pulses, can pass through the sonolucent window 810. The ultrasound waves can emanate from a MEMS array located underneath the sonolucent window 810, inside the housing of the ultrasound transducer.

FIG. 8E provides additional renderings of the exterior of the small form factor non-implantable ultrasound transducer. FIG. 8E depicts views of the exterior of the small form factor non-implantable ultrasound transducer comprising a cable 812 that can electronically and mechanically mate with the port 808 shown in FIGS. 8C and 7D. The cable 812 can receive and/or provide bidirectional data and/or power from the ultrasound transducer to an external device, such as a peripheral controller, as shown in FIGS. 13A-13C, or a computer. FIG. 8E depicts a version of the small form factor non-implantable ultrasound transducer that is manufactured according to multiple housing components. FIG. 8E depicts a sonolucent window 810, through which ultrasound waves, e.g., ultrasound pulses, can pass through the sonolucent window 810. The ultrasound waves can emanate from the MEMS array located adjacent to the sonolucent window 810, inside the housing of the ultrasound transducer. The sonolucent window 810 can comprise a biocompatible material that permits minimal acoustic impedance and minimal ultrasound attenuation, such as polymethyl methacrylate (PMMA), polyether ether ketone (PEEK), polychlorotrifluoroethylene (PCTFE), polytetrafluoroethylene (PTFE), ultra-high-molecular-weight polyethylene (UHMWPE), polyethylene terephthalate (PET), low density polyethylene (LDPE), polyether block amide (PEBAX), and/or high density polyethylene (HDPE).

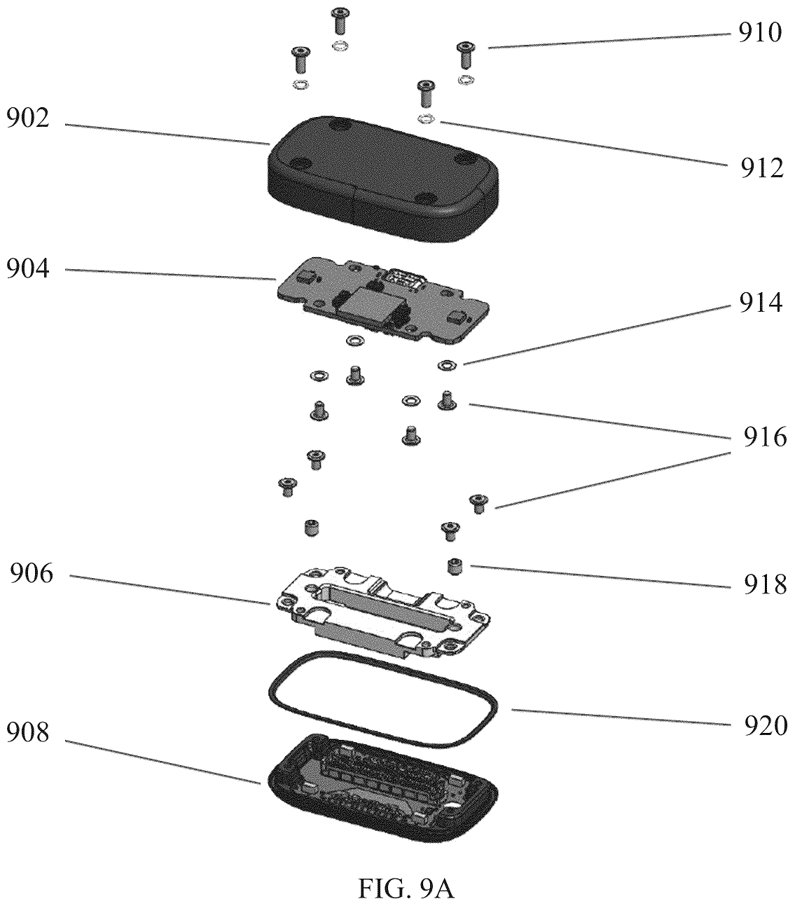

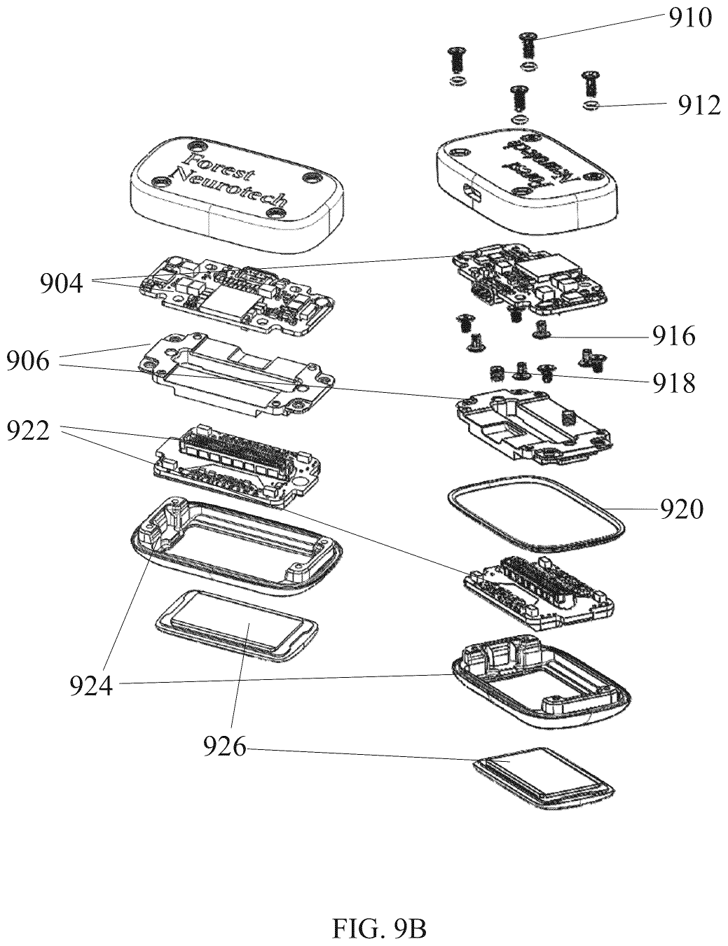

FIGS. 9A and 9B provide an exploded view rendering of the small form factor non-implantable ultrasound transducer. The parts 902-926 depicted in FIGS. 9A-9B need not describe exclusively the small form factor non-implantable ultrasound transducer, but can also describe an ultrasound transducer of any form factor, including the small and miniature form factors, and the ultrasound transducer can be implantable or non-implantable. The ultrasound transducer parts 902-926 depicted in FIGS. 9A-9B can be applicable for ultrasound transducers configured for imaging and/or modulating the nervous system of a subject. As shown in FIGS. 9A and 9B, the ultrasound transducer can comprise one or more of: an upper housing 902, which can be made of hard anodized (type III) 6061-alloy aluminum; an FPGA circuit board 904; a heat sink 906 (e.g., made of 6061-alloy aluminum); a lower housing assembly 908 comprising an ultrasound transducer; wafer head screws 910 (e.g., M2×5 mm wafer head screws made of 316 stainless steel); an O-ring 912 (e.g., made of FKM rubber); an M2 washer 914 (e.g., made of PTFE or Nylon 6); wafer head screws 916 (e.g., M2×3 mm wafer head screws made of 316 stainless steel); a screw set 918 (e.g., an M3×3 mm set screw comprising a 316 stainless steel); and a housing seal 920 (e.g., made of an elastomer).

FIG. 8B further depicts a populated circuit board 922 comprising a MEMS array, contained within the lower housing assembly 908, a housing component 924 of the lower housing assembly 908, and a sonolucent window 926 comprising a biocompatible material that permits minimal acoustic impedance and minimal ultrasound attenuation, such as PMMA, PEEK, PCTFE, PTFE, UHMWPE, PET, LDPE, PEBAX, and/or HDPE. The MEMS array comprised in the populated circuit board 922 can be a capacitive micromachined ultrasound transducer (CMUT) array built with MEMS technology, or alternatively, can be a piezo micromachined ultrasound transducer (PMUT) array, built with MEMS technology. The ultrasound transducer can be any array built with MEMS technology, and need not be limited to a CMUT or PMUT array.

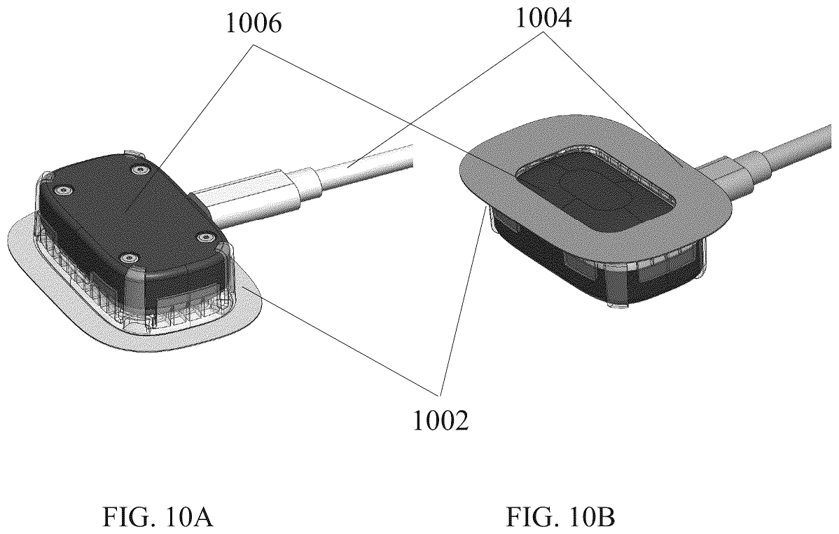

FIGS. 10A and 10B provide renderings of a version of the small form factor ultrasound transducer that is non-implantable. FIG. 10A depicts a render of the transducer that is rotated 180 degrees along a roll axis, relative to the render of the transducer shown in FIG. 10B. The transducer can comprise a brim structure 1002, which can be used to secure, e.g., adhere, the transducer against the top of a subject's scalp. That is, the brim structure 1002 secures the positioning of the transducer against the subject, given that the transducer depicted in FIGS. 10A and 10B are not implanted into the subject. The transducer can comprise a cable 1004, e.g., a USB 2 cable or any of its subversions or a USB 3 cable or any of its subversions or a cable that operates on a standardized data communication protocol or a non-standardized data communication protocol. The transducer can comprise a housing 1006, which can comprise the MEMS ultrasound array, e.g., the CMUT or the PMUT array, as well as the corresponding electronics contained within the housing.

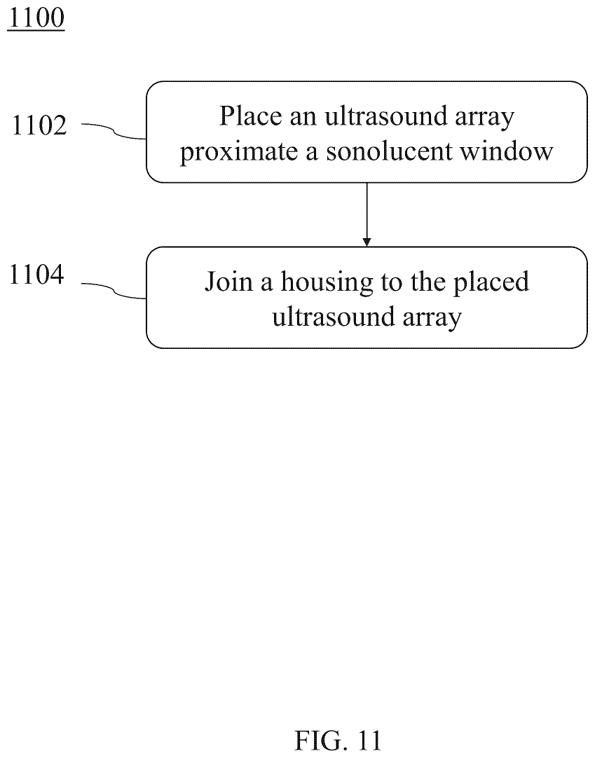

FIG. 11 depicts an exemplary workflow for assembling an implantable transducer, according to some embodiments. Steps of method 1100 can be performed, for example, using one or more electronic devices implementing a software platform and controlling a robotic apparatus. In some embodiments, at step 1102, an ultrasound array can be placed proximate a sonolucent window. In some embodiments, at step 1104, a housing can be joined to the placed ultrasound array, wherein the placed ultrasound array can be disposed, at least in part, in the housing.

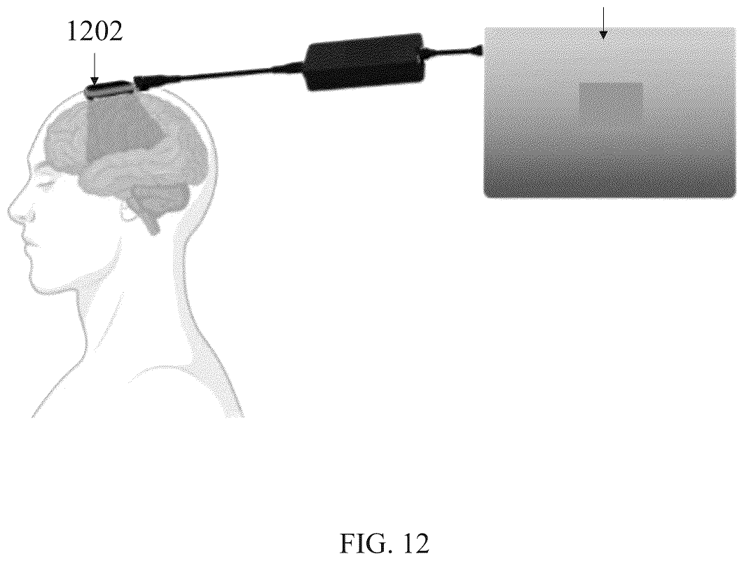

FIG. 12 provides a schematic depicting an ultrasound transducer device 1202 connected to a personal computer 1204. The schematic illustrates an implantable portion, e.g., an implantable ultrasound transducer, of the BCI system, which is connected via a cable that can handle bi-directional data transmission and unidirectional power delivery from an external device. The external device can range from custom electronics designed to program and power the implant to standard computing devices like mobile phones, tablets, and computers, any of which can enhance accessibility and ease of use.

### Peripheral Controller 120

A head mounted ultrasound neural sensing and stimulation system (e.g., macroscopic BCI system 100) may comprise multiple head implantable transducers that use a common peripheral controller for power, synchronization, and data transmission. In some embodiments, the peripheral controller can be configured to support three head implantable transducers. In some embodiments, the peripheral controller may be configured to support one to eight controllers. The number of head implantable transducers supported by the peripheral controller and used in the macroscopic BCI system is not intended to limit the scope of the disclosure and the peripheral controller.

FIGS. 13A-13C illustrate exemplary peripheral controllers. As shown in FIG. 13A, the peripheral controller 1300 may comprise, at least, a battery 1302, a wireless transmitter/receiver 1304, a DSP ASIC 1306, power management circuitry 1308, an antenna 1310 (e.g., for wireless communications), an antenna or coil 1312 for wireless charging, a connector 1314 coupling to at least one tether, and a processing unit 1316 (e.g., microcontroller or CPU). FIGS. 13B and 13C illustrate a 3-dimensional rendering of the peripheral controller 1300.

In some embodiments, the peripheral controller may not be implanted into the subject. FIG. 13B provides an exploded view of an example peripheral controller than can control one or more ultrasound transducers, e.g., non-implantable ultrasound transducers. The non-implantable peripheral controller shown in FIG. 13B can comprise a programmable push button 1318, an enclosure 1320, which can further comprise entry and exit points for a cable that can provide data and/or power, boards 1322 and 1324, which can comprise, for example, a printed circuit board (PCB), a battery, a processor, or an FPGA, and an enclosure base 1326. In the case that boards 1322 or 1324 comprise a PCB, the PCB can facilitate onboard digital signal processing (DSP), and can include one or more of the processing unit, e.g., processing unit 1316. The PCB can additionally or alternatively facilitate wireless data transmission, e.g., by incorporating the wireless transmitter/receiver 1304, and/or power management, e.g., by incorporating the power management circuitry 1308.

In some embodiments, the peripheral controller may be implanted into the subject. FIG. 13C provides an exterior view of an example implantable peripheral controller. The implantable peripheral controller can be implanted proximate to the chest cavity of the subject, similar to a pacemaker or a deep brain pulse stimulator, and thus, can be referred to as a chest unit. The implantable peripheral controller can also further comprise a power source, e.g., a battery, an enclosure, which can be made of titanium, and a PCB. The PCB can facilitate onboard digital signal processing (DSP) and can include one or more of the processing unit, e.g., processing unit 1316. The PCB can additionally or alternatively facilitate wireless data transmission, e.g., by incorporating the wireless transmitter/receiver 1304, and/or power management, e.g., by incorporating the power management circuitry 1308. In addition, a wireless charging coil can be used to charge the power source, e.g., battery, of the implantable peripheral controller, e.g., chest unit, via inductive charging. The peripheral controller, e.g., chest unit, can accommodate for the physical constraints of the subject's chest cavity, and can be within approximately 70 mm length×50 mm width×15 mm height. Alternatively, the peripheral controller dimensions can be marginally larger, such as having a length of 75 mm, 80 mm, 85 mm, 90 mm, or 100 mm; a width of 55 mm 60 mm, 65 mm, 70 mm, or 75 mm; and/or a height of 16 mm, 17 mm, 18 mm, or 19 mm. In some aspects, the peripheral controller can much smaller, such as a length of 68 mm, 66 mm, 64 mm, 62 mm, 60 mm, 58 mm, 56 mm, 54 mm, 52 mm, or 50 mm; a width of 48 mm, 46 mm, 44 mm, 42 mm, 40 mm, 38 mm, 36 mm, 34 mm, 32 mm, or 30 mm; and/or a height of 14 mm, 13 mm, 12 mm, 11 mm, 10 mm, 9 mm, 8 mm, 7 mm, 6 mm, and 5 mm, wherein the listed lengths, widths, and heights can be in any permutation to describe the dimensions of the peripheral controller. The peripheral controller can receive a tether attached to one or multiple sensing devices. In some embodiments, the peripheral controller can provide power management, as well as data transmission (e.g., configuration data, synchronization or other data) to the implantable transducers. The peripheral controller can receive data from the implantable transducers and can perform any subsequent data handling or post processing techniques before transmitting the image data to an external hub device. The external hub device can be any device that can operate as a standalone receiver, such as a device that can connect to a wireless network, such as, but not limited to, a WiFi network. In some embodiments, the external hub device can be a server, a computer, or a mobile device, such as, but not limited to, a phone or a tablet. In some embodiments, the image data may be transmitted wirelessly to the external hub device, can be connected wirelessly with the peripheral controller, and can facilitate a human interface for control of the entire system. The human interface can be intended for a user, such as, but not limited to, a clinician (i.e., the human interface can be a user interface, e.g., a clinician interface). The human interface can be configured to view or retrieve data and/or device status(es), and/or program treatment regimens and/or other functions pertaining to the use of the device.

In one or more embodiments, the peripheral controller, e.g., chest unit, can comprise biocompatible materials configured for being safely implanted in medical devices, such as, but not limited to, medical-grade polymers, titanium, or stainless steel, to prevent adverse reactions or complications within the human body. Additionally, the peripheral controller may be configured to adhere to established standards for electronics, wireless communication, and electromagnetic compatibility (EMC)—such as ISO 14708 for implantable medical devices. Adherence to these standards ensures the implant functions correctly in a wide range of conditions without interfering with other medical devices or systems.

FIGS. 14A-14B depict example implantable peripheral controllers 1404 connected to multiple implantable ultrasound transducers 1402. The set-up of an implantable peripheral controller 1404 connected to one or more implantable ultrasound transducers 1402 can comprise an implantable set-up for ultrasound-based imaging and modulating of the nervous system of a subject, and can be installed in a configuration not dissimilar to that shown in FIG. 2B.

### Peripheral Controller: DSP ASIC

In one or more embodiments, the peripheral controller can comprise a custom DSP ASIC for image processing. The custom DSP ASIC can allow for a compact and power-efficient system that can facilitate the implanting of the ultrasound transducer. The custom DSP ASIC is not necessary, however, for the implanting of the ultrasound transducer. It can perform the major image processing operations on the data received from the AFE ASIC before wireless transmission to external devices. Given that these functions can comprise highly iterative cross-functional efforts unique to the applications of the systems disclosed herein, the custom DSP ASIC can be manufactured via custom techniques, without outsourcing components of the design.

The DSP ASIC design process can comprise optimizing the image processing algorithms on an FPGA (Field-Programmable Gate Array) for rapid prototyping and testing. The FPGA-based prototyping and testing can allow for the modification and optimization of the relevant algorithms before committing them to an ASIC design. The algorithms can be programmed in a hardware description language (HDL), such as Verilog. Once the algorithms are optimized and tested on the FPGA, the algorithms can be digitally synthesized, into, for example, a physical ASIC.

To minimize power consumption and ensure efficient operation, the systems disclosed herein can comprise a small process node. Smaller process nodes can provide lower power consumption and higher transistor density, allowing for more functionality in a compact form factor and less heat generation for a given computational load. The methods and systems disclosed herein can comprise a process node of 22 nm or smaller (e.g., 20 nm, 16 nm, 14 nm, 10 nm, 7 nm, 5 nm, 3 nm, 2 nm, or 1 nm).

The design of the systems disclosed herein can incorporate various power optimization techniques, such as clock gating and power gating. Such techniques can help to minimize power consumption by selectively turning off or reducing the power to parts of the ASIC that are not in use or can operate at a lower performance level.

### Peripheral Controller: Power Management

The power management circuitry can ensure that the peripheral controller and head implantable transducers receive power from the battery and can facilitate wireless charging from an inductive charging system. Use of an inductive charging system can reduce or even eliminate the use of physical connectors, thereby diminishing the risk of infection, and ensuring a sealed and sterile environment.

The methods and systems described herein can comprise an inductive charging system for the peripheral controller. In some embodiments, the operating frequency can be within an Industrial, Scientific, and Medical (ISM) band, such as 13.56 MHz or 6.78 MHz, which can be consistent with predicate inductive charging links in medical devices. This range can provide a balance between power transfer efficiency and tissue heating, ensuring safe and effective operation. In one or more embodiments, the inductive charging system may comprise multiple-layer coils or planar coils with ferrite backing to maximize coupling efficiency while minimizing magnetic field leakage.

The methods and systems disclosed herein can further comprise optimal safety and thermal management. In some embodiments, a closed-loop temperature control system and thermal sensors are included in the chest unit to limit the temperature rise in the surrounding tissue, thereby ensuring safe operation and minimizing potential risks to the patient.

In some embodiments, the battery may comprise a lithium-ion polymer battery or a nuclear-powered battery. Based on the criteria of high energy density, long cycle life and low self-discharge rate, lithium-ion polymer (LiPo) batteries can oftentimes be a suitable battery technology. LiPo batteries can offer high energy density, long cycle life, low self-discharge rate, and are available in biocompatible versions. In some embodiments, the LiPo batteries can be molded into custom shapes to fit the implant's form factor. The methods and systems disclosed herein can comprise custom batteries matched to the system's specifications, such that a maximum capacity can be achieved, given the constrained volume. The systems described herein can comprise a battery with up to approximately 100 mAh-20 Ah of capacity (e.g., approximately 100, 200, 500, 800, 1000, 1200, 1500, 1800, 200, 2200, 2500, 2800, 3000, 3100, 3200, 3300, 3400, 3500, 3600, 3700, 3800, 3900, 4000, 5000, 800, 1000, 2000, 5000, 8000, 10000, 12000, 15000, 18000, or 20000 mAh). In the case that the peripheral controller is an implantable peripheral controller, e.g., chest unit, the battery capacity may be adjusted in accordance with the volume available in the subject's chest cavity and the energy density of the battery technology. In some embodiments, a battery technology with higher energy density can yield higher capacities, up to approximately 20000 mAh, or greater.

### Peripheral Controller: Wireless Communication

The peripheral controller may comprise a wireless transmitter and/or receiver. The choice of wireless communication technology for the peripheral controller, such as an implanted chest unit, is important. In some embodiments, ultrasound data can be used to measure brain function transferred from one implant, and can be transferred at approximately 300 Mb/s. In some examples, the B-mode ensemble for 16 2D slices of data from one implant can be estimated to be on the order of 262 Mb/s. The B-mode ensemble can refer to a collection of B-mode images that are comprised in the functional ultrasound image, where B-mode refers to the ultrasound systems' (e.g., implantable transducers) ability to send sequential ultrasound pulses in different directions to form multiple image lines, such that the sending of pulses is completed quickly and repeatedly, thereby generating an ultrasound image.

Given the described parameters, a chest unit may be configured to support up to three implants, and the wireless link can be configured to support approximately 786 Mb/s over a distance of up to 10 meters outside the body. The number of head implantable transducers supported by the peripheral controller is not intended to limit the scope of the disclosure.

The systems disclosed herein can comprise WiFi 6, implemented with a chipset like the nRF7002. WiFi 6, can deliver data rates over 1 Gbps, and can offer enhanced security features, such as WPA3 (Wi-Fi Protected Access 3) encryption, and by extension, the disclosed systems can comprise a wireless chipset that can support WPA3. The WiFi 6 chipset can also comprise energy-efficient features, such as Target Wake Time (TWT), a feature that can allow devices to negotiate when and how frequently they may wake up, to send or receive data. The use of TWT and/or other energy-efficient protocols can significantly reduce power consumption by allowing for the peripheral controller to spend more time in a low-power sleep state without compromising data transmission. Moreover, WiFi 6's advanced features, such as OFDMA (Orthogonal Frequency-Division Multiple Access) and MU-MIMO (Multi-User, Multiple-Input, Multiple-Output), can allow for efficient handling of multiple simultaneous data streams, which can further improve the energy efficiency of the system.

### Communication Across Multiple Ultrasound Transducers for Macroscale Brain Insonication

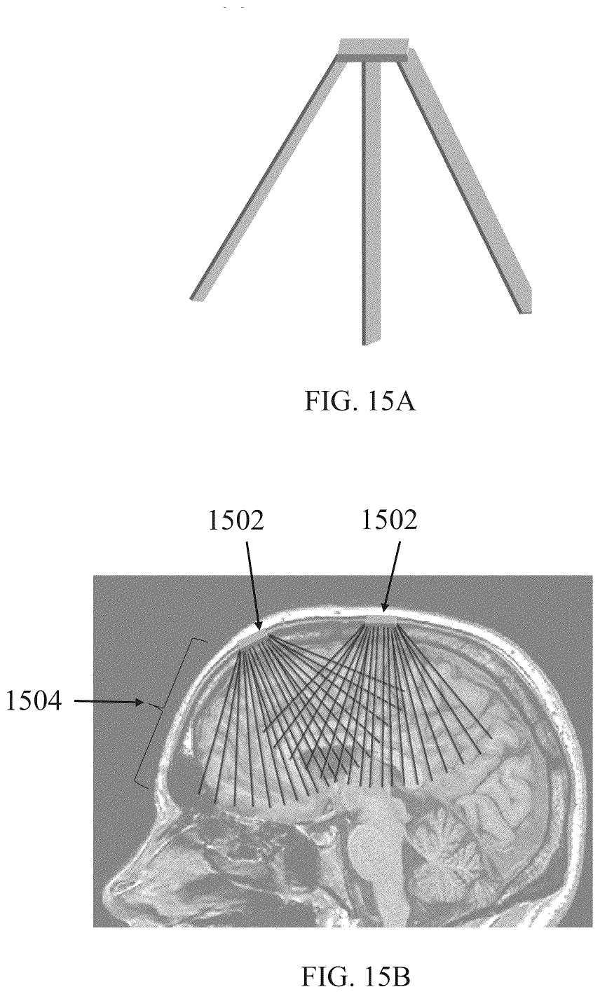

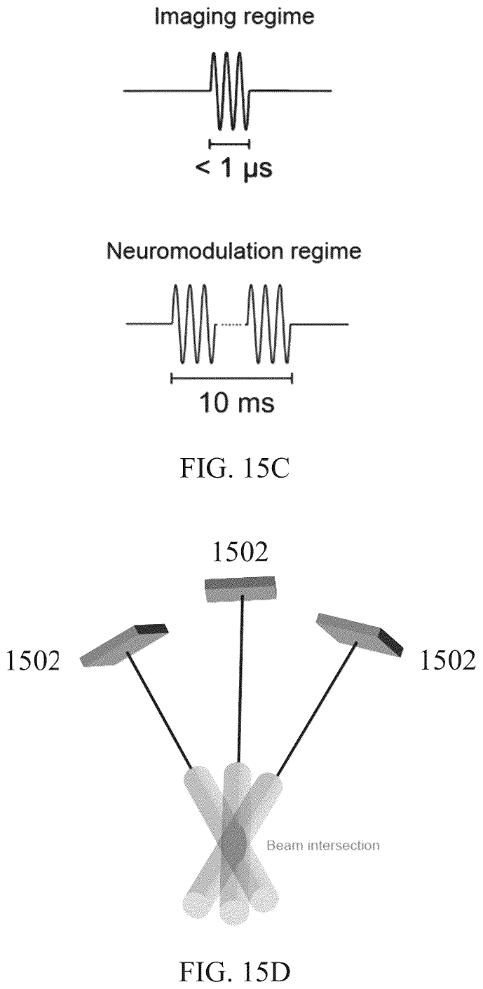

The systems and methods disclosed herein can comprise multiple implantable ultrasound transducers, such that the activities of the ultrasound transducers are coordinated, as depicted in FIGS. 15A-D. In FIG. 15A, a schematic illustration of using elevational focusing to create 2D plane waves is shown. The timing of the transmit in the elevation axis allows for the 2D planewaves to be programmably directed across a 3D volume. The three planes are shown in FIG. 15A to indicate the programmability. In FIG. 15B, a schematic illustration shows two implantable transducers 1502 emanating 2D plane waves 1504, from various hypothetical points in a patient skull. In FIG. 15C, schematic waveforms are shown to illustrate the ultrasound transmit regimes for when the implantable unit is imaging the subject, versus when the implantable unit is neuromodulating the subject. In FIG. 15D, a schematic is used to illustrate multiple implantable transducers 1502 leveraged to improve the spatial specificity and acoustic intensity at desired focal locations. The use of multiple ultrasound transducers 1502 as part of a modulatory, e.g., neuromodulatory, system can provide for better spatial specificity of the focal region as well as increased intensity through the constructive summation of the beam profiles produced by each array (in the case of three transducers 1502, the constructive interference can allow for a nine-fold increase in intensity), e.g., as shown in FIG. 15D.

The coordinated activities of the ultrasound transducers 1502 may result in an optimal system-level function, such as imaging of a distributed brain area, or neuromodulation of a target neural circuit. Orchestrating the precise activities of multiple implantable ultrasound transducers 1502 may benefit from bespoke algorithms for optimizing at least one of multiple parameters, such as, but not limited to, systems-level power consumption, imaging field of view, the magnitude of intended neuromodulation, and/or the temporal resolution of the target brain area for imaging. Although the systems described herein can image or neuromodulate a subject's physiological activity with a single ultrasound transducer alone (albeit at reduced sampling rates), embodiments for the systems and methods described herein may comprise multiple ultrasound transducers.

In some embodiments, the individual head implantable transducers may have limitations on achievable performance due to heating restrictions, power requirements, physical restrictions, or other related reasons. Using methods of synchronization of these sensor devices, by a peripheral controller, in combination with application-specific algorithms, can achieve improvements in imaging resolution and depth, as well as improvements in the delivered power per area for neuromodulation. In some embodiments, the methods of synchronization can comprise methods wherein the sensors are configured such that they have overlapping apertures. Accordingly, each sensor can individually beamform to a target within the shared focal range. The systems described herein can comprise specialized algorithms for synchronizing the sensor devices to a peripheral control device, such as an implanted chest unit. The method of synchronization may use, for example, a common timing reset signal or other configuration settings originating from the peripheral controller that can propagate to all sensor devices in the system. The method of synchronized neuromodulation may use algorithms to localize the same therapeutic point across multiple sensor devices after imaging.

Another consideration in developing application-specific algorithms for ultrasound imaging and neuromodulation within an implantable form factor is whether the available power budget given thermal dissipation constraints is sufficient to provide adequate system performance for high-impact use cases. Accordingly, embodiments in accordance with the present disclosure are designed to fall within the desired parameter ranges for thermal dissipation, as well as for physical form factor.

An ultrasound neural sensing and stimulation system (e.g., macroscopic BCI) may also encompass multiple implanted sensor devices that may be used in combination to improve the overall performance of the systems described herein. The synthesis of additional implanted sensor devices with the components of the systems described herein may benefit from bespoke integrative algorithms, wherein communication between ultrasound transducers further coordinate with the auxiliary sensor devices.

The ultrasound neural sensing and stimulation system according to embodiments of the present disclosure can be used for both imaging and neuromodulation applications. Such applications may benefit from bespoke algorithms that coordinate the functional properties of the ultrasound transducers, such that, for example, some of the transducers are configured for imaging applications, and others of the transducers may be configured for neuromodulating applications. A given ultrasound transducer may switch between being configured for imaging, to being configured for neuromodulation, or vice-versa, depending on the optimal coordination between ultrasound transducers for a given system-level task. In one or more examples of a bespoke algorithm capable of coordinating the functional properties of the implantable transducers, the prioritization of real-time monitoring with simultaneous coordinated or multi-point neuromodulation may comprise a set of ultrasound transducers that are coordinated such that some of the ultrasound transducers image, whereas other ultrasound transducers modulate. An algorithm could choose which functions each transducer performs, based on the distance between transducers and/or each transducer's aperture. The peripheral controller or external hub could enable real-time updating between the ultrasound transducers' imaging and transmission functions.

### Imaging and Modulating the Nervous System

Disclosed herein are methods and systems for imaging and modulating the nervous system. The disclosed methods and systems would overcome traditional neurotechnology shortcomings, such as ones described above. The imaging and modulating of the nervous system may operate together in a closed-loop fashion. For example, the imaging and the modulating of the nervous system may complement one another, such that a target neural activity state (e.g., for treating a nervous system disorder or disease) is achieved via iterations of imaging and modulating.

As an example, neural activity in a region of a subject's brain can be observed with ultrasound data, such as ultrasound imaging using an implantable transducer. The ultrasound data can then be analyzed, such as by a trained machine learning model, so that a set of modulation parameters (e.g., instructions for modulating the subject's neural activity) can be determined. In some embodiments, prior to the analysis, the ultrasound data are processed, as described herein. The model determines the modulation parameters, such that neuromodulations, in accordance with the determined parameters, would cause the observed region to achieve the target neural activity state. The modulation parameters may be communicated to a system for performing the neuromodulations.

The neuromodulation effects are then observed, for example, via ultrasound imaging, at selected brain regions. The differences between the observed neural activity (via ultrasound imaging) resulting from the modulation and the target neural activity state may be then analyzed by a trained machine learning model. The difference can be used to determine updated modulation parameters, such that subsequent neuromodulation, in accordance with the new parameters, can cause the subject's neural activity to converge toward the target neural activity state. When the difference between the observed activity and the target neural activity are smaller than a threshold (e.g., indicating that the observed activity sufficiently reached the target neural activity), the modulation parameters may no longer be updated.

The alternating sequence of observing the subject's neural activity followed by updating the subject's neural activity modulation can be done in real time. In some embodiments, a sequence of observing and modulating continues for a period of time based on, for example, clinical and biomedical limitations. In some aspects, each iteration of observing/analyzing the subject's neural activity and modulating the subject's neural activity based on the observations, may result in the observed neural activity being closer to the target neural activity, allowing the subject to be more efficiently and accurately treated in a minimally invasive manner and in a more individually tailored manner. In some embodiments, the time between successive iterations of observing/analyzing and modulating may depend on the condition being treated. The disclosed systems and methods may allow adjustment of this time to treat a specific condition more suitably while minimizing power consumption and the subject's commitment to treatment time. For example, for conditions such as epilepsy, the time between successive iterations may be shorter to capture sufficient data points for providing adequate instructions for modulation and treatment. For conditions such as depression, the time between successive iterations may be longer, reducing power consumption while allowing sufficient data points to be acquired for determining modulation instructions and treatment. The disclosed systems and methods may bridge a temporal gap that may complicate the fine-tuning of electrophysiological therapy. The ability to synchronize via the disclosed methods and systems can lead to more efficient and effective treatments.

Although examples of methods and systems for imaging and modulating the nervous system are described with respect to the brain, it should be appreciated that the methods and systems may be performed on other parts of the nervous system. The methods and systems can be used on other parts of the nervous system, such as non-brain parts of the central nervous system (e.g., the spinal cord) or the peripheral nervous system. Although examples of methods and systems for imaging and modulating the nervous system are described with respect to ultrasound images, it should be appreciated that the methods and systems may be performed using other kinds of ultrasound data, such as radiofrequency (RF) data.

Maladaptive conditions affecting the nervous system, such as brain disorders, are often resistant to treatment or drugs. Furthermore, the etiology surrounding conditions affecting the nervous system may be unclear. The resistance to treatment and the unclear etiologies of nervous system disorders arises, in part, due to shortcomings of traditional neurotechnologies. For example, they are limited in their resolution, scope, and flexibility when used for observing the nervous system.

Disclosed herein are methods and systems for imaging and modulating the nervous system of a subject, such that a target neural activity state in the subject is achieved for treatment of the nervous system while overcoming shortcomings of traditional neurotechnologies. The target neural activity state may be different from a neural activity state associated with maladaptive conditions of the nervous system. The methods and systems disclosed herein use ultrasound data (e.g., received via an implantable transducer) to observe physiological, e.g., neurophysiological states or neural activity, in the nervous system of the subject. The use of ultrasound data for observing neural activity in the subject provides several critical advantages over traditional methods that are described in more detail herein.

In some embodiments, the disclosed methods and systems treat the nervous system of an individual by determining instructions for modulating neural activity. The method comprises receiving, from an implantable transducer, ultrasound data of the nervous system. The ultrasound data can indicate physiological states of the nervous system, such as neurophysiological states or neural activities. The ultrasound data can be transmitted to a controller. Based on the transmitted ultrasound data, instructions for modulating neural activity in the nervous system of the individual can be determined. Neuromodulation can then be applied to the individual, based on the determined instructions protocol, for example, by transmitting the instructions to a system for performing the neuromodulation.

In some embodiments, the disclosed methods and systems observe neural activity in the context of a closed-loop observation-modulation paradigm. That is, based on the observed neural activity (e.g., via ultrasound data received from an implantable transducer, which can indicate modulation-evoked neural activity) parameters for modulating the subject are determined, such that the application of neuromodulation in accordance with the determined parameters results in the subject reaching a target neural activity state from a neural activity state at pre-neuromodulation. This closed-loop monitoring may ensure the persistent effectiveness of the therapeutic intervention.

The disclosed methods and systems may observe physiological states at a higher resolution, scope, and flexibility in a less invasive manner and in a more individually tailored manner, compared to traditional methods and systems. The present disclosure describes observing the physiological states using ultrasound data such as ultrasound imaging. The disclosed implantable transducer, system, and methods allow ultrasound data of physiological state, e.g., neural activity, which were otherwise difficult to obtain due to the described shortcomings. The use of ultrasound data allows a subject's neural activity to be observed and used to determine modulation (e.g., for example, via an algorithm or a trained model) of the subject's nervous system to treat a disorder or a disease. The disclosed systems may be configured to meet FDA guidelines and regulations while leveraging the features and advantages disclosed herein.

The use of ultrasound data for observing the subject's neural activity and performing neuromodulation in accordance with the observations provide additional advantages over traditional methods of observing modulation-evoked neural responses. Ultrasound data (e.g., ultrasound images) are capable of observing neural activity with a broader field of view, when compared to existing techniques. The broad imaging scale afforded by the ultrasound data in the context of neuromodulation is advantageous.

For example, due to the broader field of view, ultrasound data reduce a need to select a brain region for observation. The broader imaging scale afforded by ultrasound data therefore improves computational efficiency for the pipeline (e.g., improves computing efficiency of devices for determining neuromodulation instructions), because the pipeline would focus on determining the modulation parameters since ultrasound data reduce the need to select a brain region for observation.

As another example, the broader imaging scale of ultrasound data improves the accuracy of modulation instructions for achieving a target neural activity state. Some traditional methods of observing the nervous system can measure neural activity from smaller areas of the nervous system, and in doing so, risk not detecting neural activities elsewhere in the nervous system. As a result of not being able to detect the other neural activities, some traditional methods cannot provide data for more accurately determining new stimulation parameters that would cause the subject to be closer to a target neural activity state. The broader imaging scale provided by ultrasound data addresses such shortcomings.

As another example, the broader imaging scale of ultrasound data allows sensitivity to different states of the brain associated with patient symptoms, such as positive or negative mood states, tremor, or pain. This data may provide input to system (comprising a machine learning model) for identifying relevant regions of the brain and correlating them with patient moods (and used to train a machine learning model for determining instructions for treatment).

As described above, using ultrasound data of a subject's physiological state has many advantages. When combined with an appropriate neuromodulation technique (such as by using the more accurately determined information, as mentioned above), ultrasound data can more efficiently and accurately allow for a subject to achieve a target neural activity state. Examples of these neuromodulation techniques are described in more detail herein.

For example, ultrasound can also be used to perform the neuromodulation based on the ultrasound data of the subject's neural activity. Advantageously, the ultrasound-based neuromodulation can be performed using the same devices (e.g., ultrasound transducers) that receive the ultrasound data of the subject. The ability to use the same devices for both the receiving data and the modulating of the subject's nervous system increases efficiency. For example, an additional surgical procedure or cumbersome clinical setups, such as from combining the requirements of multiple neurotechnology modalities, may not be required in the case that both receiving ultrasound data and neuromodulation are performed via the same device. Examples of the disclosed methods and systems for modulating neural activity are described in more detail below.

FIG. 16A depicts an exemplary method 1600A for imaging and modulating a nervous system of a subject, for example, to treat the nervous system of a human. Steps of method 1600A can be performed, for example, using one or more electronic devices implementing a software platform. In some examples, method 1600A is performed using a client-server system, and the steps of method 1600A are divided up in any manner between the server and a client device. In some examples, the steps of method 1600A are divided up between the server and multiple client devices. Thus, while portions of method 1600A are described herein as being performed by particular devices of a client-server system, it will be appreciated that method 1600A is not so limited. In other examples, method 1600A is performed using a client device or multiple client devices. Examples of server and client devices are described in more detail herein.

In some embodiments, at step 1602A, ultrasound data of the nervous system are received. In some embodiments, the ultrasound data are received from one or more implantable transducers described herein. In some embodiments, the ultrasound data comprise one or more ultrasound images. The ultrasound image can be a two-dimensional image or a three-dimensional image.

In some embodiments, a resolution of the one or more ultrasound images, which may be received from an implantable transducer described herein, is 100 microns to 4 mm. The image resolution allows a sufficient quantity of data for determining neuromodulation instructions, as described below. This resolution may depend on imaging parameters, such as transmit frequency, device aperture, and imaging depth. For example, the full width at half max axial resolution can be described by: 1.206×λ×(z/D), where λ is wavelength of the ultrasound waves, z is imaging depth, and D is diameter of the aperture and λ=c/f, where c is the speed of sound and f is the transmit frequency. These relationships suggest that imaging resolution can be optimized based on operational mode. In some embodiments, frequencies in the range of 3 MHz to 15 MHz may be used for covering possible operational modes that can image deep into the brain, and shallow in the brain, respectively.

In some embodiments, an imaging volume of the one or more ultrasound images can be modeled as a spherical sector with a cone radius dependent on the pitch or spacing of the ultrasound elements (of an ultrasound array of an implantable transducer), relative to the transmit frequency. For example, assuming a pitch ˜λ/2 the steering angle would be 45 degrees. This gives an imaging volume that is proportional to the imaging depth as follows:

TABLE 2
Example correspondence between imaging
depth (cm) and imaging volume (cm³)

| Depth (cm) | Volume (cm³) |
|---|---|
| 5 | 76 |
| 6 | 132 |
| 7 | 210 |
| 8 | 315 |
| 9 | 447 |
| 10 | 613 |

In some embodiments, the one or more ultrasound images are received at 10 Hz to 257 kHz. For example, the frequency may be determined based on an operating frequency of a power Doppler filter. As another example, the frequency may be determined based on the speed of sound in soft tissue (e.g., 1540 m/s) and a depth associated with an image (e.g., human cortical thickness of 3 mm). In some embodiments, the one or more ultrasound images are received at 100 Hz to 25 kHz. For example, the one or more ultrasound images are received at 10 kHz. Because ultrasound acquisitions may be limited only by speed of sound and imaging depth, the disclosed methods and systems provides higher-temporal resolution for capturing information about the state of the brain, compared to other methods having more limiters, and determining neuromodulation instructions.

In some embodiments, the ultrasound data comprise radiofrequency (RF) data. In some embodiments, the RF data comprise data at frequencies of 300 kHz to 300 MHz. For example, the frequency may be twice the transmitted or received rates (e.g., Nyquist frequency of the transmitted or receive rates). In some embodiments, functional state of the brain can be determined from radiofrequency (RF) data (e.g., raw radiofrequency (RF) data) captured by implantable transducers. For example, an implantable transducer emits ultrasonic waves into the brain tissue, penetrating the tissue and subsequently reflecting back to the implantable transducer. These reflected waves induce electrical signals that can be captured as RF data, which may comprise depth-dependent echo information and respective amplitudes. This RF data may be processed as described with respect to step 1604A to estimate brain activity. In some embodiments, the RF data comprise a wide-formatted RF data (e.g., matrix), a vector of RF data, a long-formatted RF data, or any combination thereof. RF data in these formats may be received from an implantable transducer or RF data from the implantable transducer after processing.

In some embodiments, the ultrasound data can indicate physiological state of the nervous system. For example, the ultrasound data are received from one or more implantable transducers described herein. In some embodiments, the ultrasound data comprise ultrasound data captured by the implantable transducer at different points in time. For example, the ultrasound data are part of a video or a stream of ultrasound images. Additional examples of the ultrasound data are described herein.

In some embodiments, at step 1604A, the ultrasound data are processed. For example, the ultrasound data from the one or more implantable transducers can be processed at a controller. In some embodiments, the processing of the ultrasound data can comprise forming a 3D image (e.g., an image stack or volume in Cartesian X, Y, and Z dimensions), or an ordered sequence of 3D images (e.g., a volumetric video) of or relating to the nervous system of the subject. The processing of the ultrasound data can include filtering noise associated with the received ultrasound data, such as, but not limited to, one or more background subtraction steps, the application of a 2D filter, such as, but not limited to, the convolution of one or more images against a predetermined kernel (e.g., a Gaussian smoothing kernel), or inputting the one or more images into a Kalman filter, a Chebyshev filter, a Butterworth filter, a Bessel filter, a Gaussian filter, a Cauer filter, a Legendre filter, a Linkwitz-Riley filter, or any combination thereof. The filters can be applied temporally, e.g., across a plurality of images received over a plurality of timepoints. The processing of the ultrasound data at, for example, the controller, can comprise the use of one or more trained machine learning models. The processing of the ultrasound data at, for example, the controller can comprise compressing or reducing the size of the received ultrasound data, e.g., via one or more compression algorithms, such as a lossy compression algorithm (e.g., a transform coding algorithm, a color quantization algorithm, chroma subsampling, or fractal compression) or a lossless compression algorithm (e.g., run-length encoding, area image compression, predictive coding, entropy encoding, adaptive dictionary algorithms, chain codes, or diffusion models). In some embodiments, the ultrasound data is not processed and are transmitted as described below.

For example, prior to determining the instructions described herein, the ultrasound data can be processed (e.g., by the system determining the instructions, by the controller), which may include dimensionality reduction, such that a low dimensional representation, e.g., feature vectors, can be used as inputs to the machine learning algorithm.

As another example, the ultrasound data can be processed, e.g., via convolution against an imaging kernel such as an image sharpening kernel or a Gaussian blurring kernel, to improve image quality. In the case that the ultrasound data comprise a temporal sequence of ultrasound data, e.g., a video, the images in the temporal sequence can be pre-processed using background subtraction of a reference image, or by using a pre-processing analysis that considers time as a variable.

In some embodiments, the ultrasound data comprise radiofrequency (RF) data, and one or more ultrasound images are generated based on the RF data. For example, prior to digitization, processing steps can occur in the analog domain. Initially, the RF data (e.g., reflected ultrasound signals) can be amplified using a low-noise amplifier. These signals may also pass through a band-pass filter to eliminate unwanted noise or frequencies outside the desired range. Variable-gain amplifiers may be employed to dynamically adjust amplification levels, compensating for attenuation effects that arise due to variations in tissue depth. To prevent aliasing artifacts during digital sampling, an anti-aliasing filter may be applied to the analog signals. These pre-digitization procedures prepare the analog signals for efficient and accurate conversion into a digital format.

In some embodiments, the RF data are digitized. Once digitized, the RF data undergo further processing to create images. For example, the RF signals are converted to baseband through IQ demodulation. The demodulated signals can be subjected to low pass filtering and subsequently downsampled to reduce the data volume. Then, beamforming techniques can be applied. In Delay-and-Sum (DAS) beamforming, planewave IQ data from multiple transducer elements can be temporally aligned and combined to focus the ultrasound signals at different points within the imaging field. This time alignment can be determined based on the time it takes for an ultrasound wave to travel from the transducer to a specific focal point and back. The aligned IQ data can then be summed to create a single, beamformed signal for each focal point. This process can be reiterated for multiple focal points to generate either 2D or 3D images.

In some embodiments, to determine the functional state of the brain, the received ultrasound data (e.g., RF data) undergo clutter filtering. In some embodiments, clutter filtering is configured to distinguish dynamic changes, such as blood flow, from stationary or slow-moving tissue signals. In some embodiments, clutter filtering comprises methods such as high pass filtering or Singular Value Decomposition (SVD), which is configured to isolate physiologically relevant signals within, e.g., the brain.

In some embodiments, at step 1606A, the processed ultrasound data are transmitted. For example, the processed ultrasound data are transmitted to a controller. In some embodiments, the controller is a separate device in communication with the one or more implantable transducers. For example, the controller may be a client device such as an intermediate device for communicating the processed ultrasound data to a second device (e.g., a device comprising an algorithm or a model) for determining instructions for neuromodulation. As another example, the controller may be part of a device or system that determines instructions for neuromodulation. In some embodiments, the controller is integrated with the one or more implantable transducers.

The controller can be configured to receive data associated with physiological state and/or data associated with the neural activity. The physiological state can comprise neurophysiological state, and the neurophysiological state can comprise hemodynamic activity. The hemodynamic activity can be indicated by power Doppler intensity (PDI) values. Power Doppler is a technique that uses the amplitude or intensity of Doppler signals (e.g., from ultrasound data from the implantable transducer) to detect moving matter, such as the flow of blood, e.g., hemodynamic activity. For example, changes in the PDI values can be proportional to changes in the subject's hemodynamic activity, such as the subject's cerebral blood volume (CBV) activity (e.g., CBV signals). Therefore, changes in PDI values from the ultrasound data can be used to determine CBV activity. The physiological state can comprise non-neural activity representing neural activity. For example, CBV signals are indicative of vascular changes related to neural activity, such that variations in neural activity can be represented by monitoring variations in CBV signals. That is, CBV signals, such as those acquired via ultrasound data, are a function of neural activity signals. Neurophysiological state can comprise hemodynamic activity such as CBV signals derived from ultrasound data, because the CBV signals are indicative of neural activity patterns, e.g., the firing patterns of neurons in the nervous system of the subject.

In some embodiments, the ultrasound data or the processed ultrasound data are transmitted for storage or training a machine learning model. For example, the ultrasound data and information associated with a neural activity state of the ultrasound data are stored or used for training a machine learning model (e.g., for determining a neural activity state, for determining instructions for neuromodulation, as described in more detail herein).

In some embodiments, at step 1608A, instructions for modulating neural activity of the nervous system are determined based on the processed ultrasound data. For example, a second device or system is configured to receive the processed ultrasound data and determine the instructions for modulating the neural activity. For instance, a device comprising an algorithm, or a model receives the processed ultrasound data (e.g., from the one or more implantable transducers, from the controller) and determines the instructions, via the algorithm or the model, based on the images. As another example, a processor of the one or more implantable transducers determines these instructions. In some embodiments, the instructions are determined further based on the additional data, such as data associated with physiological state and/or data associated with the neural activity. Additional examples of the instructions and determination of the instructions are described herein.

Modulating the neural activity can comprise stimulating one or more regions of the nervous system. The one or more regions of the nervous system being stimulated can comprise one or more regions of a peripheral nervous system, one or more regions of a central nervous system, or a combination thereof. The regions of the peripheral nervous system can include nerve cells related to the somatic system or the autonomous system, such as, but not limited to, cranial nerves (e.g., the vagus nerve), spinal nerve, and motor neurons. The regions of the central nervous system can comprise a spinal cord. One or more regions of the central nervous system can comprise a brain. Stimulating the one or more regions of the nervous system can comprise electrical stimulation via one or more electrodes and the instructions for the modulating the neural activity can comprise electrical stimulation via one or more electrodes. In some embodiments, the electrical stimulation is controlled via electrical modulation parameters comprising amplitude, frequency, pulse width, intensity, waveform, polarity, acoustic pressure, or any combination thereof. The electrical stimulation can comprise deep brain stimulation (DBS), transcranial magnetic stimulation (TMS), repetitive TMS (rTMS), vagus nerve stimulation (VNS), transcranial direct current stimulation (tDCS), electrocorticography (ECoG), optogenetics, or any combination thereof.

Examples of the stimulation are described here. Deep Brain Stimulation (DBS) is a surgical technique used for movement disorders like tremor associated with Parkinson's Disease, essential tremor, and limited non-motor applications, e.g., obsessive compulsive disorder. DBS involves implantation of electrodes in specific brain areas to regulate abnormal impulses through electrical signals. Stereo Electroencephalography (sEEG) can be used for primarily monitoring neural activity associated with intractable forms of epilepsy. Electrodes are implanted into the brain to record electrical activity and localize the source of seizures. This information can then be used to plan surgical ablation or resection. sEEG and DBS combined approaches can include implanting both recording and stimulation electrodes. Transcranial Magnetic Stimulation (TMS) and Repetitive Transcranial Magnetic Stimulation (rTMS) are non-invasive technologies that can use a magnetic field to stimulate specific areas of the brain. rTMS is used to treat depression and other psychiatric disorders. Vagus Nerve Stimulation (VNS) involves a device implanted under the skin that can send electrical signals to the brain via the vagus nerve, commonly used to treat epilepsy and depression. Transcranial Direct Current Stimulation (tDCS) is a non-invasive form of brain stimulation that uses constant, low current delivered via electrodes on the head. Electrocorticography (ECoG) can involve placing electrodes directly on the exposed surface of the brain to record electrical activity. sEEG and DBS may be combined to both record and stimulate electrodes. Stereo-EEG Responsive Neural Stimulation (RNS) is a neurostimulation system developed for refractory focal epilepsy. RNS monitors brain activity and provides stimulation when abnormal patterns are detected. Structural Connectivity Imaging for DBS Lead Placement uses high-resolution imaging to guide DBS lead placement. Due to the advantages of ultrasound data described herein, the disclosed methods and systems allow these stimulation systems to be more efficiently and accurately operated (e.g., in response to the instructions determined via the methods described herein) in a less invasive manner for treating a nervous system disorder or disease.

In some embodiments, the instructions are used to cause a system for performing neuromodulation (and treating a nervous system disorder or disease). Examples of systems for performing the neuromodulation (e.g., systems for stimulation, the one or more ultrasound transducers) are described herein. In some embodiments, the instructions comprise adjusting electrical modulation parameters, spatial modulation parameters, temporal modulation parameters, or any combination thereof. The parameters may be advantageously individualized based on the subject.

The electrical modulation parameters can comprise amplitude, frequency, pulse width, intensity, waveform, polarity, acoustic pressure, or any combination thereof. The amplitude can be the voltage or current level of an electrical pulse. The amplitude can be the intensity of an electrical current, e.g., measured in milliamperes (mA). The frequency can be the rate at which the electrical pulses are delivered, e.g., measured in Hertz (Hz). The frequency can also the frequency of an ultrasound wave, e.g., in the range of 0.2-10.0 MHz for neuromodulation. The pulse width can be the duration of each electrical pulse, e.g., measured microseconds (s). The intensity can be the strength of the magnetic field, e.g., expressed as a percentage of the maximum output of the device or relative to the subject's motor threshold. The intensity can be the power of an ultrasound wave, e.g., measured in watts per square centimeter (W/cm²). The waveform can be the shape of the magnetic pulse, which can be monophasic or biphasic. The polarity can refer to the direction of current flow, determined by the placement of the anode and cathode electrodes. Anodal stimulation can generally excite neuronal activity, whereas cathodal stimulation can inhibit neuronal activity. Acoustic pressure can refer to the amount of pressure extended by an ultrasound wave.

The spatial modulation parameters can comprise electrode configuration, electrode position, electrode size, electrode placement, directionality, coil orientation, coil position, stimulation focality, stimulation bilaterality, montage, focus size, target location, or any combination thereof. The electrode configuration can refer to the choice of which one or more contacts can be adjusted, when using implanted electrode systems, which may have multiple contacts. The electrode configuration can involve the arrangement of multiple electrodes, which may be used in more advanced or experimental setups. The electrode position can refer to the location within the target brain region where the electrode is placed. The electrode position can be adjusted during initial surgical implantation of one or more neuromodulation technologies. The directionality can refer to the directional steering of the electrical field. The coil orientation can refer to the angle at which a coil is held relative to the scalp. The coil orientation can affect the directionality of the induced current. The coil position can refer to the specific area of the brain being targeted, often guided by neuro-navigational systems. The stimulation focality can refer to coils that are designed to provide more focal stimulation, in contrast to coils that provide broader stimulation. The stimulation bilaterality can involve stimulating both hemispheres either simultaneously (e.g., such that the start of stimulating one hemisphere can be before ending the stimulation of the other hemisphere) or in an alternating fashion. The electrode size can refer to the surface area of the electrodes, which can impact the current density, and consequently, the effects of stimulation. The electrode placement can refer to the location of an anode and cathode on the scalp or other body parts, e.g., guided by the 10-20 EEG system, or other methods. The montage can refer to the specific combination of electrode size and placement for targeting specific brain regions. The focus size can refer to the dimensions of the area where an ultrasound wave is focused, which can affect the specificity of the neuromodulation. The target location can refer to the location or plurality of locations within the brain that the ultrasound waves can be focused on.

The temporal modulation parameters can comprise bursting, cycling, ramping, frequency, pulse duration, train duration, inter-train interval, total number of pulses, stimulation patterning, duration, inter-stimulus interval, session frequency, pulse repetition frequency, duty cycle, or any combination thereof. The bursting can refer to systems that allow for pulses to be delivered in bursts rather than continuously. The bursting can also refer to a burst mode, which can refer to some protocols that deliver pulses in groups or bursts with intra-burst frequency and inter-burst intervals as additional parameters. The cycling can refer to systems that can be programmed to turn on and off at set intervals. The cycling can also refer to some protocols that can involve periods of stimulation interspersed with periods of no stimulation. The ramping can refer to the gradual increase or decrease in amplitude over a specified time period. The ramping can also refer to the gradual increase or decrease in a current amplitude at the beginning or end of the session, to minimize discomfort in the subject. The ramping can also refer to the gradual increase or decrease in intensity or acoustic pressure over a specified time period to minimize potential side effects in a subject. The frequency can refer to the rate at which pulses are delivered, e.g., measured in Hertz (Hz). The pulse duration can refer to the length of a magnetic pulse. The pulse duration can also refer the length of time an ultrasound pulse lasts, e.g., measured in milliseconds (ms). The train duration can refer to the length of time over which a series of pulses (i.e., a train) is delivered. The inter-train interval can refer to the time between separate trains of pulses. The total pulses can refer to the total number of magnetic pulses delivered during a session. The patterned stimulation can refer to some protocols which use more complex patterns of stimulation, such as theta-burst stimulation (TBS), which can involve bursts of pulses at specific frequencies. The duration can refer to the length of time a current is applied, e.g., measured in minutes. The inter-stimulus interval can refer to protocols where multiple sessions are applied, and the time between the end of one session and the start of the next session can be referred to as the inter-stimulus interval. The session frequency can refer to how often sessions are conducted, e.g., daily or weekly. The pulse repetition frequency can refer to the rate at which pulses are emitted, which may be measured in Hertz (Hz). The duty cycle can refer to the fraction of time that the ultrasound is active within a given period, expressed as a percentage.

The instructions for the modulating the neural activity can be determined based on a target neural activity. The target neural activity may be received by a device for determining the instructions for the neuromodulation. The target neural activity may be compared with the subject's current neural activity to determining, e.g., whether the neuromodulation instructions should be modulated, whether neuromodulation can cease. The target neural activity can be determined via ultrasound data (e.g., received from the one or more implantable transducers), fMRI imaging, electrophysiological recordings, structural magnetic resonance imaging scans, diffusion tensor imaging (DTI), computed tomography (CT) scan information, or any combination thereof. For example, this information is received by a device for determining the target neural activity.

The target neural activity can be determined via the processed ultrasound data (e.g., processed version of ultrasound data received at step 1602A). The target neural activity can be determined based on an output of a transfer learning algorithm. In some instances, the target neural activity can be determined based on a transfer learning algorithm (e.g., as described herein) trained on data types associated with various brain observing modalities, such as electrophysiological timeseries traces and/or image sequences, such as volumetric image sequences.

The target neural activity can be expressed according to one or more terms, which advantageously allow comparison between the observed neural activity (e.g., via the processed version of ultrasound data from the one or more implantable transducers) and the expression of the target neural activity for determining the neuromodulation instructions. The target neural activity can be expressed as a composite time-independent state. That is, the composite time-independent state can be a collapsing, e.g., an averaging, of multiple snapshots of neural activity across subjects and/or across time. The target neural activity can be expressed as a multi-dimensional timeseries data. For example, rather than a single static composite state, the target neural activity can be expressed, for instance, as a video, or an ordered sequence of matrices or arrays. The observed neural activity state can be said to be achieving the target neural activity state, if the two multi-dimensional timeseries can relate to one another via a statistical technique for comparing timeseries, such as, but not limited to, a cross-correlation matrix, a cross-correlation function, a cross-variance matrix, a cross-variance function, an ARIMA model, or a multiply-trended regression model. The multi-dimensional timeseries data can comprise temporal resolution or spatial resolution equal to or less than those of the ultrasound data. That is, the target neural activity state can have a lower resolution than that of the ultrasound data of the subject's neural activity.

The instructions for modulating the neural activity can be determined based on an output of a machine learning algorithm. The output of the machine learning algorithm can be based on the processed ultrasound data (e.g., transmitted from step 1606A) provided to the machine learning algorithm.

The machine learning algorithm can comprise reinforcement learning, Bayesian optimization, a generalized linear model, a support vector machine, a deep neural network, representation learning, or any combination thereof. The machine learning algorithm can be trained offline, tested offline, or validated offline. These offline operations may be not performed in real-time. That is, offline can refer to not taking place immediately after the acquisition of new data, such as the acquisition of new ultrasound data. In some aspects, training, testing, or validating the machine learning algorithm, offline, can entail training, testing, or validating the machine learning algorithm, independently and/or in parallel from the acquisition and some analyses of new data (e.g., such that the start of acquiring new data can happen prior to when analyzing existing data stops), strengthening the model for determining the neuromodulation instructions via a different source. The machine learning algorithm can also be trained, tested, or validated, online. In some embodiments, the training improves the machine learning algorithm's ability to categorize between neural activity states (e.g., healthy brain state, not healthy brain state) for determining the instructions for neuromodulation. In some embodiments, the machine learning model is trained via the processed versions of ultrasound data received from the implantable transducers. In some embodiments, the machine learning model is trained via a mapping between an instruction for the neuromodulation and a brain state responding to the neuromodulation (e.g., such that the machine learning model would better understand a nervous system response to a particular neuromodulation instruction).

As another example, the machine learning model comprises a decision engine, which may be a machine learning model configured to interpret the ultrasound data to recommend appropriate neuromodulation parameters. These steps can be performed in real-time, and the neuromodulation device can be updated accordingly. As another example, patient-reported outcomes or real-time biofeedback can be used to fine-tune the recommendations made by the machine learning model. As another example, the machine learning model can integrate ultrasound data with data from other methodologies, such as electrophysiology methods, including, but not limited to, EEG, ECoG, or depth electrodes, and/or imaging methods like fMRI, fNIRS, or microscopy, to create a more comprehensive neural activity map, which can contribute to more effective neuromodulation.

In some embodiments, the instructions include a region of the neural activity being modulated, and the region can be determined based on the processed ultrasound data (e.g., by a device described above). For example, the processed ultrasound data can comprise an ordered sequence of images that are ordered with respect to time, such as a video or a stream of ultrasound images. The processed ultrasound data can comprise an ordered sequence of images that are ordered with respect to space, such as an X-, Y-, or Z-axis in a Cartesian space. That is, the processed ultrasound data can be used to generate volumetric depictions of the subject's nervous system. For example, of series 2D images along the X- and Y-axes can be stacked to generate a volume along a Z-axis. The resulting volume can make up for missing images along the Z-axis, such that unexpected skips along the Z-axis do not preclude the construction of the ultrasound imaging volume.

As an example, if the locations for DBS electrode implantation are not yet determined, the one or more implantable transducers capture ultrasound data of the brain for determining these locations. For instance, the one or more implantable transducers capture macro-scale brain network activity patterns across multiple frequencies and brain regions, thereby establishing a comprehensive functional connectome. For example, to determine the DBS electrode implantation locations, the functional data obtained from this process are combined with structural MRI scans and diffusion tensor imaging (DTI) based structural connectomics and would guide the precise stereotactic placement of DBS electrodes (e.g., by providing the ultrasound, MRI, and DTI data to a machine learning model).

In some embodiments, a model, such as a machine learning model, can be used to infer parts of the ultrasound imaging volume. The ultrasound imaging volumes can be a volumetric timeseries (e.g., recorded at 0.5 Hz, 1 Hz, 2 Hz), e.g., a video comprising of images corresponding to data in X, Y, and Z dimensions, such that a volume comprising an ordered stack of 2D images exists for one or more ordered timepoints. The machine learning model can also be used to infer parts of ultrasound data that are not ultrasound images, such as RF data.

The region of the neural activity being modulated can comprise an anatomical part of the nervous system. For example, the region of the nervous system can comprise, at least in part the hippocampus, or the amygdala. It should be appreciated that the region of the nervous system need not directly correspond to an anatomical region of the nervous system associated with a neuroanatomical label. For example, the determined region of the nervous system can overlap across many labeled neuroanatomical parts of the nervous system.

The instructions for modulating the neural activity can be determined further based on pre-trial physiological state information, such as physiological state information prior to performing imaging and modulation. The pre-trial physiological state information may be transmitted to the device for determining the instructions for modulating the neural activity.

The pre-trial physiological state information can comprise pre-trial ultrasound information, functional magnetic resonance imaging (fMRI) information, electrophysiological recordings, structural magnetic resonance imaging scans, diffusion tensor imaging (DTI) information, computed tomography (CT) scan information, or any combination thereof. A machine learning model (e.g., the machine learning model for determining the neuromodulation instructions) can be trained via the pre-trial physiological state information. Additional examples of pre-trial physiological state information are described with respect to second physiological state information below.

During the early adoption of ultrasound data in observation-modulation paradigms, adequate example data, e.g., training data, comprising the same data type that is natively outputted from the ultrasound imaging, e.g., ultrasound data, may be less available for making accurate predictions of modulations parameters. Advantageously, the pre-trial information allows the model to be more accurate when ultrasound image training data are less available. In such cases, a model, such as a machine learning model, can be trained on the additional data types (which can also include pre-trial ultrasound data) such that the algorithm can learn to infer from data types that it has not been trained on in abundance, such as ultrasound data from the implantable transducers when they are less available, to predict a set of parameters for modulating the subject's nervous system.

In some embodiments, a transfer learning-based algorithm can be used such that a lack in, e.g., ultrasound data from the implantable transducers need not preclude the use of a machine learning model to determine modulation parameters from observed neural activity. The transfer learning model can be adjusted, such that ultrasound image training data can be weighted more over non-ultrasound data, during the training of the transfer learning model. The machine learning model, such as the transfer learning algorithm, can comprise one or more machine learning techniques, such as, but not limited to reinforcement learning, Bayesian optimization, a generalized linear model, a support vector machine, or a deep neural network.

In some embodiments, the instructions for the modulating the neural activity are communicated to a system for performing the neuromodulation. For example, a device determining the instructions for modulating the neural activity communicates (e.g., directly transmits, communicates via a second device) the instructions to a system for performing the neuromodulation. Examples of these neuromodulation systems are disclosed herein. In some embodiments, an interfacing device, a protocol, or both can be used to allow communications with the neuromodulation system. The communications may comprise communication of the neuromodulation instructions, for example, determined via step 1608A.

In some embodiments, the instructions for modulating the neural activity are transmitted to a neuromodulation system via an interfacing device. In some embodiments, the interfacing device is part of the controller described herein. For example, the interfacing device is a universal translator module (UTM). In some embodiments, the UTM is a hardware component that can act as a translator between an ultrasound interface (e.g., associated the processed ultrasound data, associated with ultrasound data from an implantable transducer) and the neuromodulation system. It can receive raw or processed ultrasound data, convert the data into a format compatible with the neuromodulation system, and send modulating instructions in the compatible format to the neuromodulation system.

As another example, the interfacing device is a gateway server. In some embodiments, the central gateway server is configured to store communication protocols associated with different neuromodulation systems. The disclosed system may send data to the gateway server, which then can translate the received data and forward appropriate instructions to the neuromodulation system.

As another example, components or ASICs (Application-Specific Integrated Circuits) of the disclosed system (e.g., an implantable transducer, a controller) could be programmed with communication protocols to interface directly with different neuromodulation systems.

In some embodiments, the instructions for modulating the neural activity are transmitted to a neuromodulation system via a communications protocol. As an example, the communications protocol comprises API-level communication. For instance, a set of APIs is developed to enable the disclosed system to communicate with the neuromodulation systems. This API may be configured by manufacturers of the neuromodulation system to be compatible with the disclosed system. As another example, the communications protocol comprises IoT communication protocols, such as MQTT, CoAP, or HTTP/HTTPS for real-time data communication. As another example, the communications protocol comprises wireless communication standards (e.g., Bluetooth, WiFi), creating a seamless, cable-free connection between devices.

In some embodiments, the communications protocol comprises a security protocol for protecting the integrity and confidentiality of the neural data. For example, the security protocol comprises end-to-end encryption. The ultrasound data and the neuromodulation instructions may comprise sensitive data. End-to-end encryption may be implemented for data transmission to protect the sensitive data from being received by an unwanted party. As another example, the security protocol comprises an authentication protocol. The authentication protocol may comprise using authentication methods to ensure connections between authorized devices.

Methods of closed-loop neuromodulating a subject's neural activity based on observed, e.g., imaged neural activity, where at least one of the neuromodulating or the observing of the neural activity is achieved with ultrasound-based technology, e.g., focused ultrasound for the neuromodulating, and can comprise iteratively imaging and modulating the subject's neural activity, such that the observed neural activity resembles a target neural activity pattern, e.g., target neural activity state.

FIG. 16B depicts an exemplary method 1600B for imaging and modulating a nervous system of a subject, for example, to treat the nervous system of a human. Steps of method 1600B can be performed, for example, using one or more electronic devices implementing a software platform. In some examples, method 1600B is performed using a client-server system, and the steps of method 1600B are divided up in any manner between the server and a client device. In some examples, the steps of method 1600B are divided up between the server and multiple client devices. Thus, while portions of method 1600B are described herein as being performed by particular devices of a client-server system, it will be appreciated that method 1600B is not so limited. In other examples, method 1600B is performed using a client device or multiple client devices. Examples of server and client devices are described in more detail herein.

In some embodiments, at step 1602B, ultrasound data of the nervous system is received from an implantable transducer, wherein the ultrasound data can indicate physiological state of the nervous system. In some embodiments, at step 1604B the ultrasound data of the nervous system can be processed. In some embodiments, at step 1606B, the processed ultrasound data can be transmitted, wherein the instructions for modulating neural activity of the nervous system are determined based on the processed ultrasound data. In some embodiments, at step 1608B the instructions for the modulating the neural activity can be received by the implantable transducer. In some embodiments, at step 1610B, the ultrasound neuromodulation can be performed on the subject by the implantable transducer.

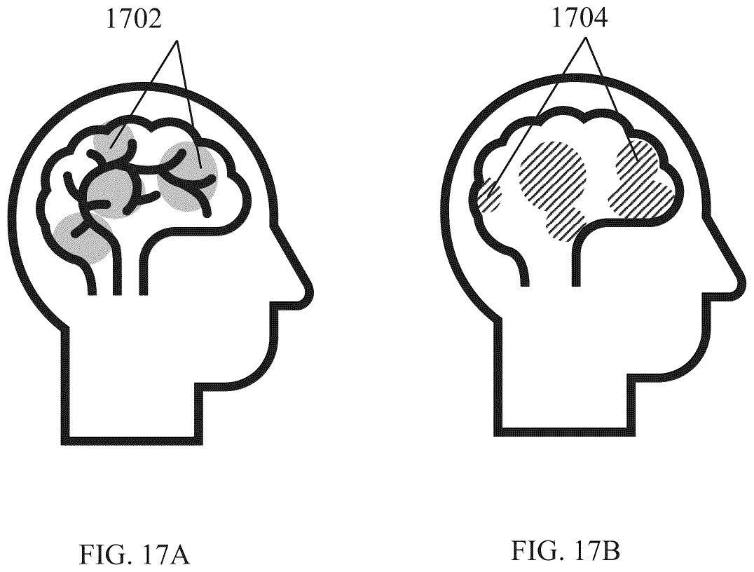

FIGS. 17A and 17B depict two exemplary schematics representing neural activity states, e.g., brain activity states. The brain activity states depicted in FIGS. 17A and 17B show schematic heat maps illustrating spatial and/or temporal patterns regions 1702 of the brain (FIG. 17A), and a different spatial and/or temporal pattern regions 1704 of the brain. In FIG. 17A, the brain activity denotes a healthy brain state. The healthy label, or positive valence, associated with the healthy brain state can be determined by correlating one or more observed brain states to one or more known indicators of a healthy physiological state, such as one or more behaviors. Observed brain states that correlate to the one or more known indicators of a healthy physiological state, e.g., one or more healthy behaviors, can be considered to be an example of one or more healthy brain states.

In FIG. 17B, the brain activity denotes a maladaptive brain state. The maladaptive label, or negative valence, associated with the maladaptive brain state can be determined by correlating one or more observed brain states to one or more known indicators, e.g., one or more behaviors, of a maladaptive physiological state, e.g., a brain disorder or disease. Observed brain states that correlate the one or more known indicators of a maladaptive physiological state, e.g., one or more disease behaviors, can be considered to be an example of one or more maladaptive brain states.

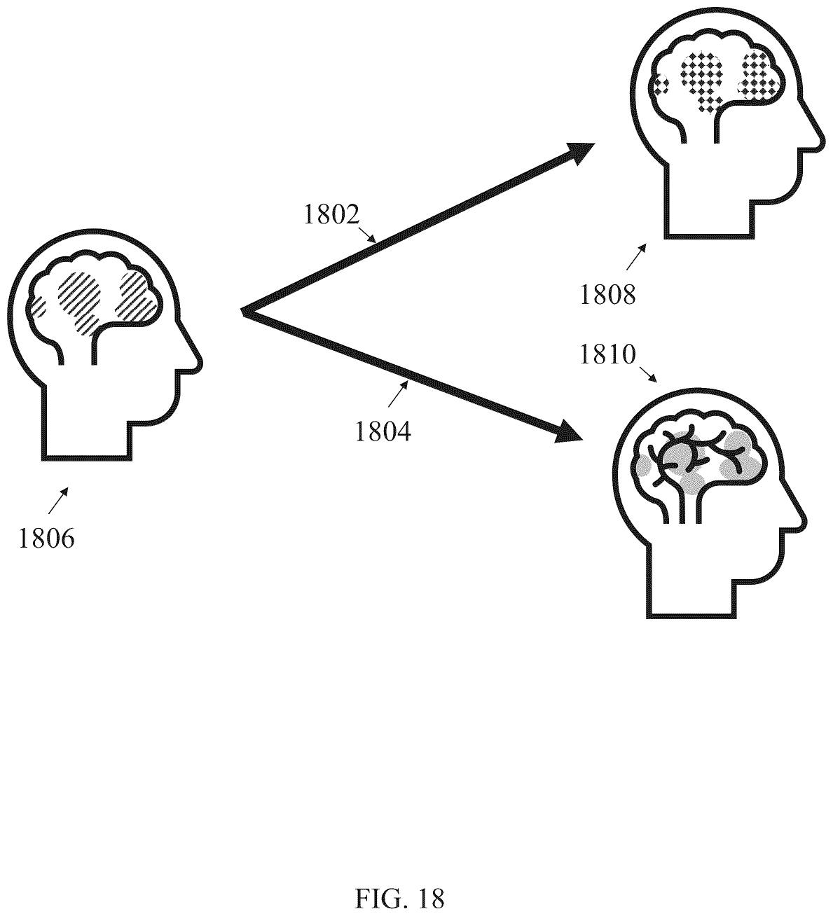

FIG. 18 depicts exemplary schematics of an initial brain activity state 1806 and two example new brain activity states in response to neuromodulation in accordance with two different instructions (e.g., neuromodulation set A 1802 corresponding to first instructions, neuromodulation set B 1804 corresponding to second instructions). FIG. 18 illustrates a brain's responses 1808 and 1810 to different neuromodulations, and the responses (and the associated neuromodulation) may be used to train a model for determining neuromodulation instructions. This data may allow quantitative evaluation of how different neuromodulation parameters modulate brain activity and, subsequently, influence affective states.

As illustrated, neuromodulation set A 1802 can result in changes in brain activity, when comparing the new brain activity state 1808 and the initial brain activity state 1806. Neuromodulation set B 1804 can result in different changes in brain activity, when comparing the new brain activity state 1810 and the initial brain activity state 1806. The methods and systems disclosed herein aim to direct an initial brain activity state towards a desired or target brain activity state. The determination of modulation parameters is important for directing the subject's brain activity state towards the target brain activity state. Described herein are algorithms that can predict a set of neuromodulation parameters that can efficiently direct a subject's brain activity state towards a target brain activity state. Due to the advantages of ultrasound data described herein, the disclosed methods and systems allow neuromodulation to be more efficiently and accurately performed (e.g., in response to the instructions determined via the methods described herein) in a less invasive manner for treating a nervous system disorder or disease.

As an example, FIG. 18 depicts a schematic representing brain activity states in response to different neuromodulations, such as DBS electrodes or implantable ultrasound transducers. Upon implantation of the DBS electrodes or the implantable ultrasound transducers, the ultrasound system can monitor the brain's functional network response (e.g., via ultrasound data received from one or more implantable transducers) to variations in neuromodulation parameters. For DBS-based neuromodulation, the parameters for modulation may include modulations in pulse amplitude, pulse width, frequency, duty cycle, and precise stereotactic location within a brain region being monitored. For ultrasound-based neuromodulation, the parameters for adjusting the ultrasound-based neuromodulation can comprise parameters including intensity, frequency, acoustic pressure, focus size, target location, pulse duration, pulse repetition frequency, duty cycle, burst mode, cycling, and ramping. The intensity can refer to the power of the ultrasound wave, which can be measured in watts per squared cm (W/cm²). The frequency can refer to the frequency of the ultrasound wave, which can range between 0.2-10.0 MHz for ultrasound-based neuromodulation of the subject. The acoustic pressure can refer to the amount of pressure exerted by the ultrasound wave. The focus size can refer to the dimensions of the area where the ultrasound wave is focused, which can affect the specificity of the neuromodulation. The target location can refer to the location or plurality of locations within the brain that the ultrasound waves are focused on. The pulse duration can refer to the length of time each ultrasound pulse lasts, and can be measured in milliseconds (ms). The pulse repetition frequency (PRF) can refer to the rate at which the pulses are emitted, which can be measured in Hertz (Hz). The duty cycle can refer to the fraction of time that the ultrasound is active within a given period, and can be expressed as a percentage. The burst mode can refer to a protocol of delivering ultrasound waves which can deliver the ultrasound wave pulses in groups or “burst” with intra-burst frequency and inter-burst intervals as additional parameters. The cycling can refer to intervals of ultrasound application followed by periods of no application. The ramping can refer to a ramping up or ramping down, where a gradual increase or a gradual decrease in intensity or acoustic pressure over a specified time period to minimize potential side effects. The alterations in the brain network activity induced by these changes can constitute a dataset. These data can permit the quantitative evaluation of how different DBS parameters modulate brain activity and, subsequently, influence affective states.

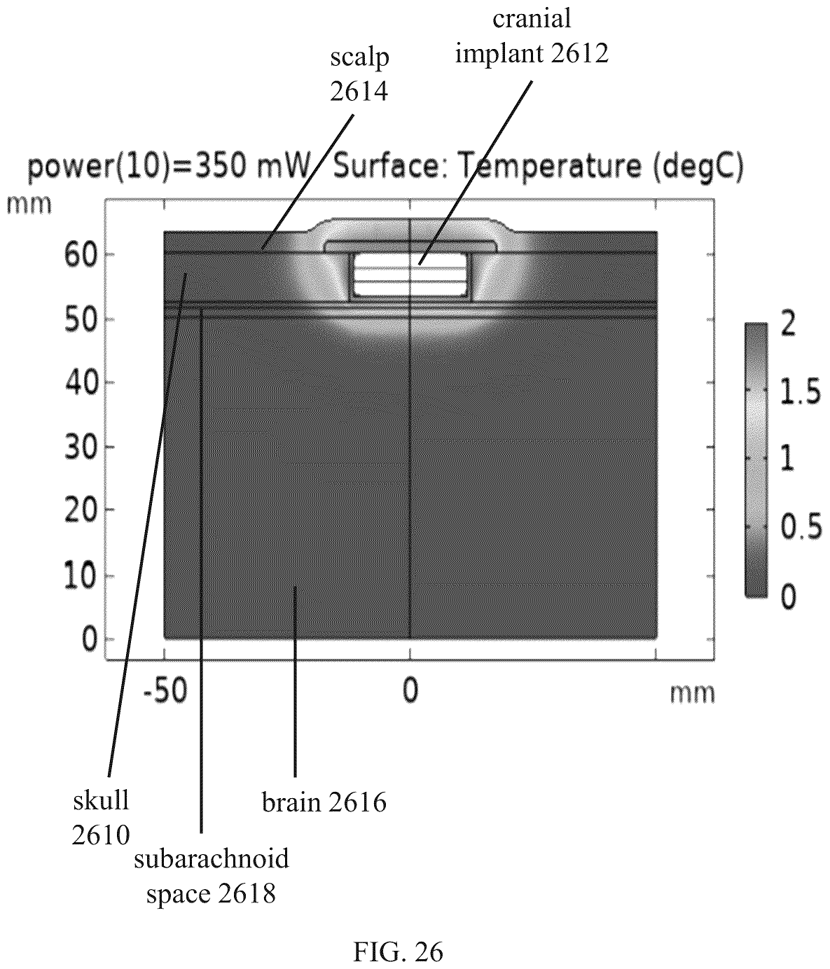

In some embodiments, parameters are determined based on a library of previously effective parameters. In some embodiments, the parameters can be determined as part of an algorithmic approach (e.g., Bayesian Optimization, a machine learning algorithm disclosed herein) designed to identify optimal parameters efficiently within a large search space (as described with respect to FIG. 26). These parameters are communicated to a neuromodulation system (e.g., a programmable DBS system). Given the potential time-varying and non-linear relationship between DBS parameters, brain network activity, and individual variability, this process may comprise iterative fine-tuning to ensure that the determined neuromodulation parameters are uniquely tailored to optimize patient outcomes.

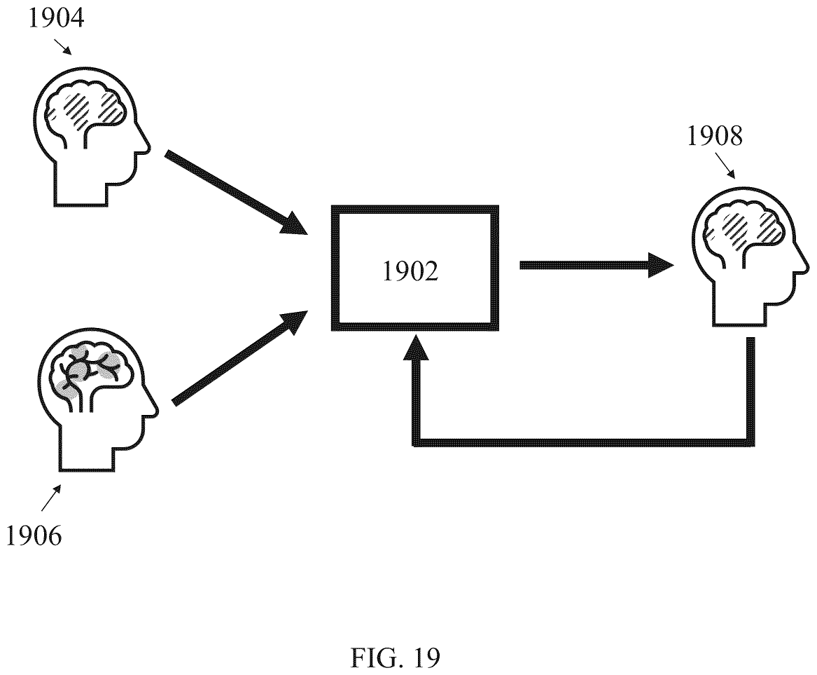

FIG. 19 depicts an exemplary schematic depicting adjustment of neuromodulation parameters to achieve desired brain state. For example, system 1902, which may comprise a system disclosed herein, analyzes a brain state 1904 associated with a neural activity (e.g., a current brain state determined via ultrasound data received from an implantable transducer) and a target brain state 1906 associated with a target neural activity (e.g., determined as described herein). Based on the brain state 1904 and the target brain state 1906, the system 1902 (e.g., an algorithm of the system) determines neuromodulation parameters (e.g., stimulation parameters, instructions for neuromodulation), and applies a neuromodulation protocol, in accordance with the neuromodulation parameters. The neuromodulation-evoked brain activity state 1908 is then measured with ultrasound data, as described herein. The neuromodulation-evoked brain activity state is then analyzed by the system 1902 in a feedback, such that the difference between the neuromodulation-evoked brain activity state 1908 and the target brain state 1906 is determined. Based on the difference, an adjusted set of neuromodulation parameters is determined, such that the subject's observed brain state can better achieve the subject's target brain state 1906.

### Closed-Loop Neuromodulation Tools

The closed-loop neuromodulation tools can comprise: real-time monitoring, which can promote the ability to monitor brain response in real time during the neuromodulation. The monitored brain response can be achieved by continuous or intermittent acquisition of functional ultrasound data. The closed-loop neuromodulation tools can comprise feedback control algorithms, which can adjust the neuromodulation parameters in real time based on the monitored brain response, with the goal to maintain or induce certain brain states; and/or adaptive neuromodulation tools, which can comprise tools for supporting adaptive neuromodulation, where the neuromodulation parameters are dynamically adjusted across sessions based on the subject's response history.

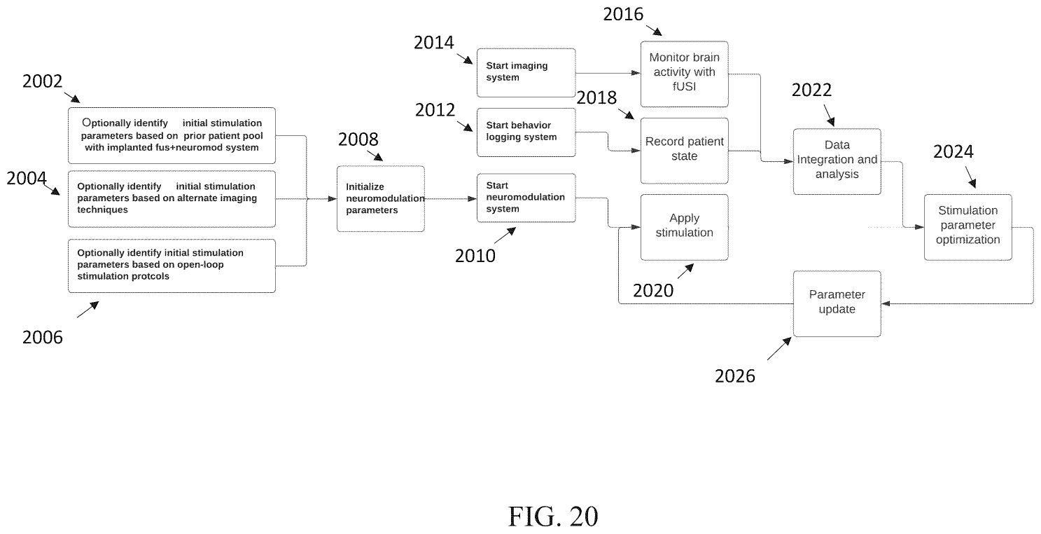

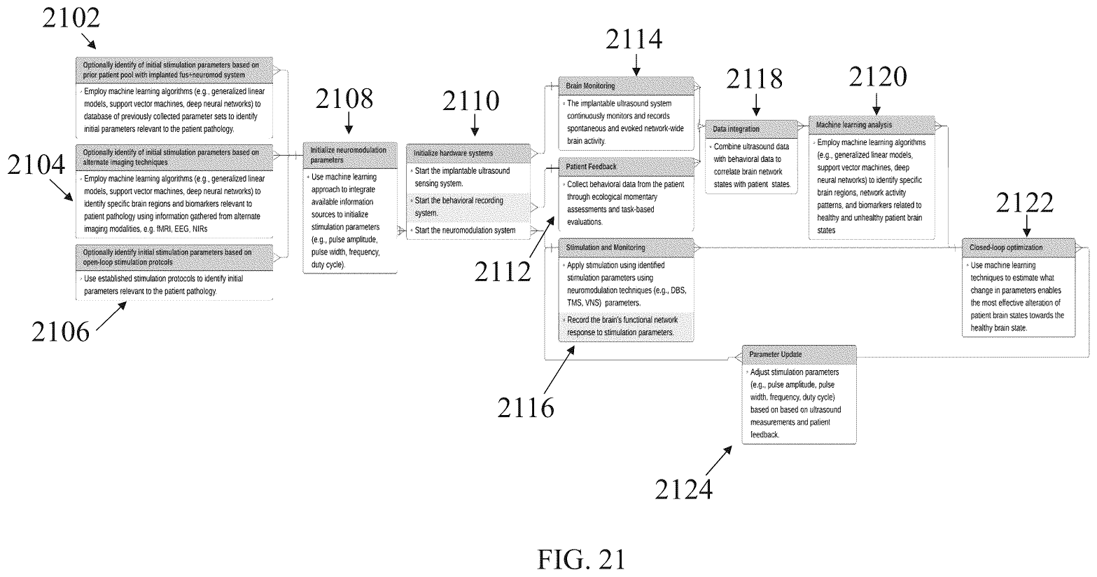

FIG. 20 and FIG. 21 provide flowcharts for processes 2000 and 2100, that can describe the junctions at which closed-loop neuromodulation tools can be implemented. For example, step 2002 or 2102, where an initial stimulation is optionally identified based on a prior patient pool, the optional identification can be performed via a software tool. In addition, for step 2004 or 2104, where the initial stimulation is optionally identified based on alternate imaging techniques, the initial stimulation can be identified via a software tool. In addition, for step 2006 or 2106, where the initial stimulation is optionally identified based on open-loop stimulation protocols, the initial stimulation can be identified via a software tool. For step 2008 or 2108, the software tool can initialize the neuromodulation parameters. For step 2010 or 2110, the neuromodulation system can be started via a software tool. For step 2012 or 2110, the behavior logging system can start via a software tool. For step 2014, the imaging system can start via a software tool. For step 2016 or 2114, the neural activity, such as the neural activity of the brain, can be monitored with focused ultrasound, and the monitoring can be coordinated and logged via a software tool. For step 2018 or 2112, the patient state can be recorded via a software tool that can stream and/or store the patient state. For step 2020 or 2116, a stimulation can be applied, via a software tool. For step 2022 and/or 2118, the data can be integrated and/or analyzed via a software tool. The software tool for the integration and/or analysis of the data can be done offline, e.g., on an external server, such as a third-party server, such as an Amazon Web Services- or Microsoft Azure-based server. The analysis of the neural imaging data can inform the optimization of parameters (e.g., step 2024, 2120, or 2122) for neuromodulating a subject, and the optimization can be done via a software tool. The neuromodulating can, but need not be limited to, ultrasound-based neuromodulating, e.g., the neuromodulating can be electrophysiology-based. For step 2026 or 2124, the optimized parameters for neuromodulating the subject based on the subject's neural recordings can be used to provide updated neuromodulation, e.g., stimulation, parameters to the subject.

It should be appreciated that steps described with respect to FIG. 20 and/or FIG. 21 are exemplary. The method 2000 and/or 2100 may include fewer steps, additional steps, or different order of steps than described. It is appreciated that the steps of method 2000 and/or 2100 leverage the features and advantages described above with respect to other figures.

In some embodiments, the method 2000 and/or 2100 is part of a nervous system treatment. For example, the modulation of neural activity caused by instructions determined via the method 2000 and/or 2100 is for the nervous system treatment. The treatment may comprise treating, without limitation, chronic pain, depression and anxiety, compulsion disorder, Parkinson's Disease, essential tremor, epilepsy, post-traumatic stress disorder, a memory disorder, or any combination thereof. The compulsion disorder can be obsessive compulsive disorder, substance abuse disorder, or both.

### Closed-Loop Ultrasound-Based Neuroimaging and Ultrasound-Based Neuromodulating

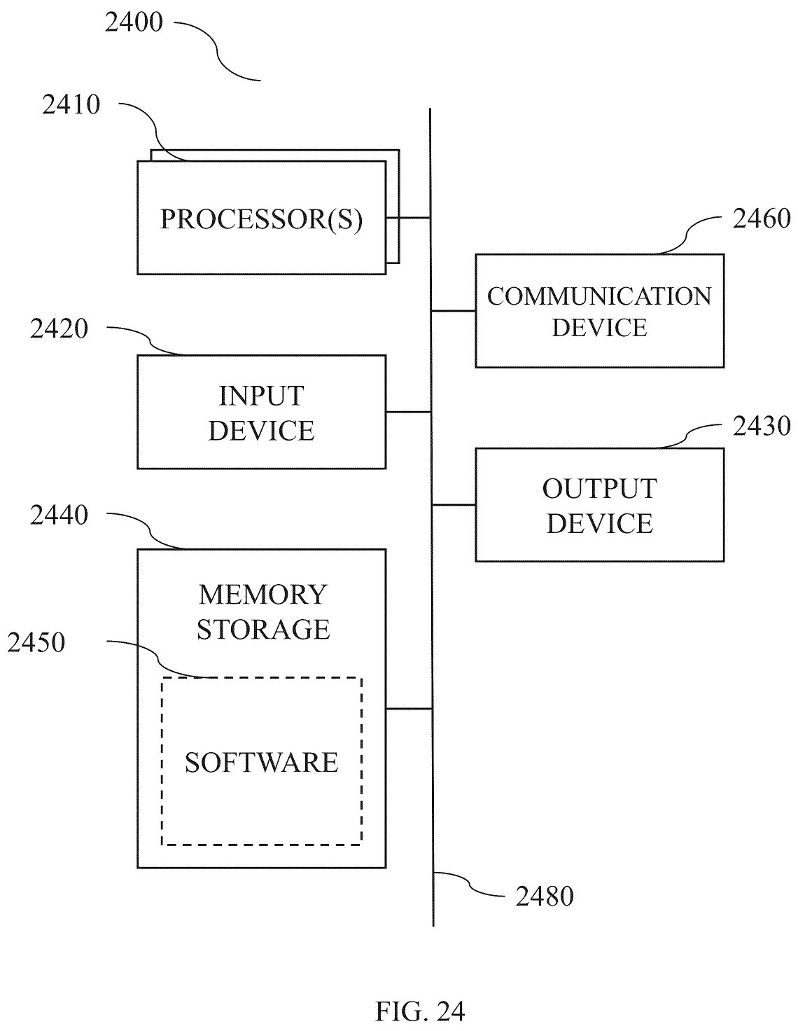

In some examples, the closed-loop method of modulating and imaging of the nervous system, including the brain of the subject, to achieve a target neural activity state, can comprise imaging neural activity from the nervous system via ultrasound-based neuroimaging, e.g., via ultrasound transducers, and modulating neural activity from the nervous system via ultrasound-based neuromodulating, e.g., via ultrasound transducers. That is, an iterative ultrasound imaging and ultrasound neuromodulating protocol (e.g., an “ultrasound-ultrasound” protocol) can be used on the subject. For example, the ultrasound-ultrasound protocol can comprise an ultrasound transducer that can be acutely placed either epidurally inside the skull, just outside the dura mater—e.g., an implantable ultrasound transducer as depicted, for example, in at least FIGS. 2A, 2B, and 24—or non-invasively on the scalp via an ultrasound transducer resembling the renderings depicted in FIGS. 10A and 10B. The flexibility of an ultrasound-exclusive system—e.g., an imaging and modulating system free of electrophysiological implements—can include temporarily deploying the ultrasound transducers across a broad range of time scales, ranging from a few minutes to up to 30 days, and thereby catering to a broad spectrum of diagnostic and therapeutic needs. The implantable portion of the system can be connected via a cable that handles bi-directional data transmission and unidirectional power delivery from an external device. The external device can range from custom electronics specifically designed to program and power the implant, to standard computing devices such as mobile phones, tablets, and computers, thereby enhancing accessibility and ease of use. A system comprising exclusively ultrasound-based technologies for both the imaging and modulating of the nervous system can serve a wide array of neurological functions, ranging from monitoring and diagnostic purposes to therapeutic interventions across various neurological disorders and injuries.

Algorithms for Imaging with Multiple Ultrasound Transducers

The methods and systems described herein can address the development of advanced imaging algorithms for ultrasound-based medical devices with a plurality of transducers. These algorithms can optimize the performance and precision of ultrasound imaging and neuromodulation arrays by carefully structuring array characteristics, including center frequency, channel counts, array geometry, bandwidth, and angular sensitivity. In one or more embodiments, these algorithms can be implemented in a custom digital signal processor (DSP) chip, a field programmable gate array (FPGA), or in a peripheral hub device. The methods and systems described herein leverage the synthesis of the array parameters with novel imaging sequences and a comprehensive range of simulation results, while also integrating effects such as skull geometry, phase correction, transducer position optimization, acoustical intensity, and estimates of effective treatment volume. Additionally, the methods and systems described herein employ distinct imaging strategies which can comprise plane-wave, focused, and diverging-wave approaches, while factoring in hardware design constraints and power requirements. The described methods and systems can comprise ‘pitch-catch’ algorithms, which can allow transmission on multiple ultrasound transducers, while receiving from others, overlapping or separate. In some implementations, the described methods and systems can comprise standard imaging algorithms, e.g., a delay-and-sum algorithm, where a delay can be added to implantable unit's transduction, such that the signals from a particular direction or implantable unit are aligned before they are summed. The feasibility of such methods as described herein is further supported by results from non-limiting exemplary simulations of the ultrasound imaging sequence, which can cover the entire brain volume using small, implantable ultrasound devices suitable for burr-hole surgeries. The methods and systems described herein employ steering arrays and ultrasound beams to large angles in azimuth and elevation. In doing so, the ultrasound-based methods and systems disclosed herein achieve extensive coverage of the subject's target biological area for study.

Analyzing Ultrasound Imaging Data from Ultrasound Transducers

Disclosed herein are methods and systems of analyzing ultrasound imaging data from ultrasound transducers, including implantable ultrasound transducers. The imaging data being analyzed can include, for example, raw radiofrequency data, raw in-phase and quadrature (IQ) data, beamformed IQ data, and intensity values based on the beamformed IQ data. The imaging data can include anatomical, e.g., non-functional, ultrasound imaging data, e.g., B-mode imaging data, and/or functional ultrasound imaging data, e.g., power Doppler imaging data.

The processing of raw radiofrequency data for functional ultrasound imaging can involve any one of several operations that transform data, and any of the operations can be performed in software, and/or as part of an algorithm. For example, the raw radiofrequency data generated from ultrasound transducers can be converted into in-phase and quadrature (IQ) data, which can be converted into beamformed IQ data, which can be converted into brightness mode (B-mode) images, which can be used to determine magnitudes of power Doppler data, which can be equivalent to the intensity values representing changes in cerebral blood volume, which can be correlated to neural activity data, and can be visualized. These data transformations also relate to the formation of compound images. That is, in planewave imaging, such as ultrasound imaging, multiple images are formed by transmitting acoustic energy into the medium at different angles. To form a compound image, backscattered echoes from the transmitted acoustic energy are received, and the backscattered echoes are then separately beamformed and summed together. The magnitude of the image can then be computed and log-compressed to form the B-mode image. An ensemble of compound images can then be filtered to extract the power Doppler image, such that noise such as motion data deriving from non-blood motion, e.g., tissue motion, can be parsed from the blood motion, which can represent cerebral blood volume changes, and by extension, neural activity.

Algorithms, including algorithms implemented on a computer, e.g., software, can be used to perform more granular processing steps, such as any of the processing steps involved in transforming the beamformed IQ images into B-mode images. Given that B-mode images are derived from the complex values of the beamformed IQ images, software for analyzing ultrasound data as discussed herein, can be involved in any of the following three general operations for converting IQ images into B-mode images: a) beamforming; b) magnitude determining; and/or c) brightness mapping. During the beamforming of IQ images, the received ultrasound echoes can be aligned and summed to form the complex IQ image, which can comprise both amplitude and phase information from the ultrasound waves. During the magnitude determining process, the IQ image can comprise complex values representing the IQ components of the signal. The magnitude of complex values can be determined using the formula:

$$magnitude = \sqrt{{I}^{2} + {Q}^{2}}$$

where I is the in-phase component and Q is the quadrature component of the signal.
During the brightness mapping, the calculated magnitude values can then be mapped to a grayscale intensity to form the B-mode image. When visualizing the B-mode image, higher magnitude values can correspond to brighter pixels, which can represent stronger ultrasound echoes. In contrast, lower magnitude values can correspond to darker pixels, which can represent weaker ultrasound echoes.

### Artificial Neural Network Reconstruction of Functional Ultrasound Images

Existing methods for functional ultrasound imaging rely on singular value decomposition (SVD) to transform an array of B-mode or IQ images into estimates of blood flow. This technique, although effective, demands extensive computational resources due to the necessity of acquiring and processing large volumes of data, typically on the order of 200 to 400 images. Such demands can inherently limit the temporal resolution and elevate the power consumption of imaging systems.

In contrast, modern deep learning approaches indicated herein can potentially reduce the requisite number of B-mode images, by up to 95%, by training models to emulate the outputs produced by SVD algorithms. Furthermore, the actual quantity of interest can extend beyond the static maps that represent the outputs of the SVD and can instead include the vascular map's dynamic changes over time, which are crucial for understanding brain function.

The methods described herein can include an artificial neural network (ANN) that significantly refines the use of deep learning in functional ultrasound by modifying the cost function of the ANN, such that the cost function prioritizes sensitivity to dynamic changes in brain function. Modifying the cost function can be achieved by incorporating a behavioral correlation metric that quantifies the relationship between neural stimuli and corresponding vascular responses. This metric can guide the model to detect subtle, functionally relevant fluctuations within the cerebral vasculature that are indicative of neural activity. In addition, the cost function can include terms that optimize for how well the functional images can predict simultaneously acquired functional data.

The functional ultrasound data for training the ANN can be captured during controlled behavioral tasks known to induce specific neural responses, ensuring that the vascular changes are both predictable and relevant. The ANN model can then be trained using the modified cost function which can include a novel term reflecting the statistical correlation between the measured vascular response and the behavioral stimulus. Training of the ANN model can be optimized to prioritize minor yet functionally significant variations in pixel intensity over larger, less informative variations, ensuring that the model is finely tuned to detect functional changes.

The use of the ANN to reconstruct functional ultrasound images, such as power Doppler images, provides several advantages. For example, the ANN can provide improved functional sensitivity over conventional methods, such as SVD-based reconstruction of power Doppler images. By focusing on the detection of small-scale changes within vascular maps that correspond to neural activities, the ANN model can offer unparalleled sensitivity in functional imaging. In addition, the ANN can provide improved efficiencies in data usage, relative to the conventional SVD-based methods. The ANN can reduce the number of images, e.g., B-mode images or compound images, needed for reconstructing a functional ultrasound image, e.g., a power Doppler image, thereby lowering the computational load and enabling higher frame rates for real-time imaging applications. Furthermore, by reducing the computational load used for reconstructing a power Doppler image based on the B-mode images, the power consumption used for reconstructing the power Doppler image can be correspondingly reduced, when compared to the conventional methods, such as SVD. The use of the ANN can also directly integrate the subject's behavioral data during training of the ANN or during deployment of the trained ANN. The direct integration of the behavioral data into the imaging process can allow for more precise mapping of the regions of relevant neural activity for the subject. The improved efficiencies in data requirements and power consumption can translate to direct benefits in the clinic. Clinically, use of the ANN can offer potential for real-time monitoring treatments, which can provide immediate feedback on therapeutic efficacy, and improved diagnostic accuracy for neurological conditions.

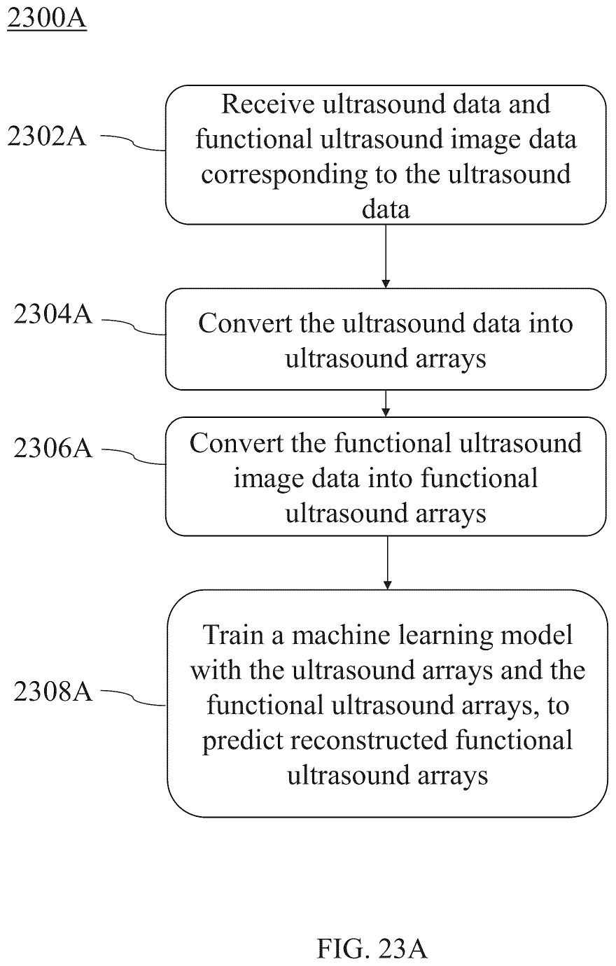

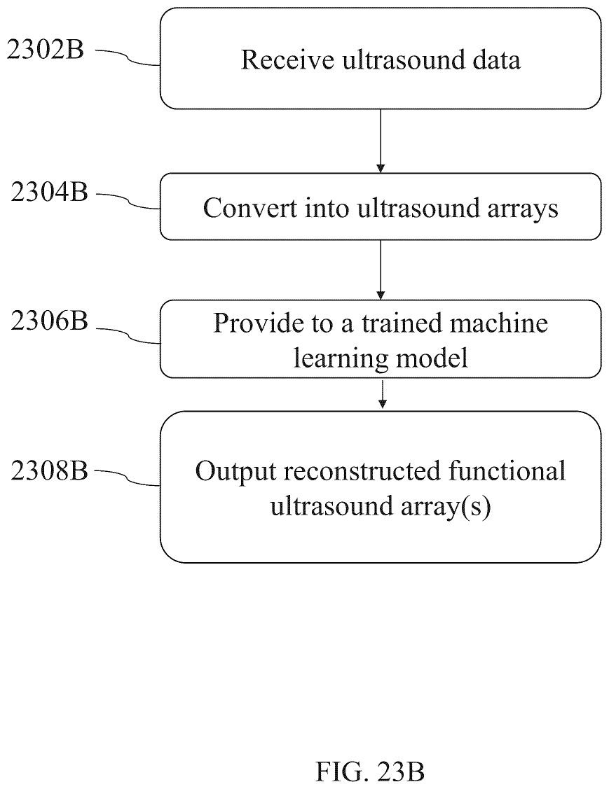

FIG. 23A depicts an exemplary method 2300A for training machine learning model for reconstructing one or more functional ultrasound images. Steps of method 2300A can be performed, for example, using one or more electronic devices implementing a software platform. In some examples, method 2300A is performed using a client-server system, and the steps of method 2300A are divided up in any manner between the server and a client device. In some examples, the steps of method 2300A are divided up between the server and multiple client devices. Thus, while portions of method 2300A are described herein as being performed by particular devices of a client-server system, it will be appreciated that method 2300A is not so limited. In other examples, method 2300A is performed using a client device or multiple client devices. Examples of server and client devices are described in more detail herein.

In some embodiments, at step 2302A, one or more ultrasound data from one or more samples from one or more subjects, obtained from an implantable transducer, and one or more functional ultrasound image corresponding to the one or more ultrasound data, is received. In some embodiments, at step 2304A, the one or more ultrasound data is converted into one or more ultrasound arrays. In some embodiments, at step 2306A, the one or more functional ultrasound image data is converted into one or more functional ultrasound arrays. In some embodiments, at step 2308A, the machine learning model is trained with the one or more ultrasound arrays and the one or more functional ultrasound arrays, to predict one or more inferred functional ultrasound arrays from inputted one or more ultrasound data or inputted one or more ultrasound arrays. The ultrasound arrays or the functional ultrasound arrays can be ND (n-dimensional arrays) and can comprise a matrix or a tensor of any dimension. The converting the ultrasound data into the ultrasound array can comprise multiplying each element of the ultrasound data by 1, which can, but not necessarily, for example, change the computational object type of the ultrasound data, in the case that the ultrasound data is computationally instantiated. Similarly, the converting the functional ultrasound image data into the functional ultrasound arrays can comprise multiplying each element of the functional ultrasound image data by 1, which can, but not necessarily, for example, in the case that the functional ultrasound image is computationally instantiated. The ultrasound data or the functional ultrasound data can already be formatted as an array, prior to the converting, in which case, the converting can comprise any operation that maintains the array format of the ultrasound data or the functional ultrasound data.

FIG. 23B depicts an exemplary method 2300B for reconstructing a functional ultrasound image from anatomical ultrasound images. Steps of method 2300B can be performed, for example, using one or more electronic devices implementing a software platform. In some examples, method 2300B is performed using a client-server system, and the steps of method 2300B are divided up in any manner between the server and a client device. In some examples, the steps of method 2300B are divided up between the server and multiple client devices. Thus, while portions of method 2300B are described herein as being performed by particular devices of a client-server system, it will be appreciated that method 2300B is not so limited. In other examples, method 2300B is performed using a client device or multiple client devices. Examples of server and client devices are described in more detail herein.

In some embodiments, at step 2302B, one or more ultrasound data from one or more samples is received from one or more subjects. In some embodiments, at step 2304B, the one or more ultrasound data is converted into ultrasound arrays. In some embodiments, at step 2306B, the one or more ultrasound arrays can be provided to a trained machine learning model. In some embodiments, at step 2308B, one or more inferred functional ultrasound arrays can be outputted, based on the received one or more ultrasound data. The ultrasound arrays or the functional ultrasound arrays can be ND (n-dimensional arrays) and can comprise a matrix or a tensor of any dimension. The converting the ultrasound data into the ultrasound array can comprise multiplying each element of the ultrasound data by 1, which can, but not necessarily, for example, change the computational object type of the ultrasound data, in the case that the ultrasound data is computationally instantiated. Similarly, the converting the functional ultrasound image data into the functional ultrasound arrays can comprise multiplying each element of the functional ultrasound image data by 1, which can, but not necessarily, for example, in the case that the functional ultrasound image is computationally instantiated. The ultrasound data or the functional ultrasound data can already be formatted as an array, prior to the converting, in which case, the converting can comprise any operation that maintains the array format of the ultrasound data or the functional ultrasound data.

Any of a variety of machine learning approaches & algorithms (where a machine learning model, as referred to herein, comprises a trained machine learning algorithm) may be used in implementing the disclosed methods, as the ANN configured to reconstruct one or more functional ultrasound images from anatomical ultrasound images. For example, the machine learning model may comprise a supervised learning model (i.e., a model trained using labeled sets of training data), an unsupervised learning model (i.e., a model trained using unlabeled sets of training data), a semi-supervised learning model (i.e., a model trained using a combination of labeled and unlabeled training data), a self-supervised learning model, or any combination thereof. In some examples, the machine learning model can comprise a deep learning model (i.e., a model comprising many layers of coupled “nodes” that may be trained in a supervised, unsupervised, or semi-supervised manner).

In some instances, one or more machine learning models (e.g., 1, 2, 3, 4, 5, 6, 7, 8, 9, 10, or more than 10 machine learning models), or a combination thereof, may be utilized to implement the disclosed methods. In some instances, the one or more machine learning models may comprise statistical methods for analyzing data. The machine learning models may be used for classification and/or regression of data. The machine learning models can include, for example, neural networks, support vector machines, decision trees, ensemble learning (e.g., bagging-based learning, such as random forest, and/or boosting-based learning), k-nearest neighbors algorithms, linear regression-based models, and/or logistic regression-based models. The machine learning models can comprise regularization, such as L1 regularization and/or L2 regularization. The machine learning models can include the use of dimensionality reduction techniques (e.g., principal component analysis, matrix factorization techniques, and/or autoencoders) and/or clustering techniques (e.g., hierarchical clustering, k-means clustering, distribution-based clustering, such as Gaussian mixture models, or density-based clustering, such as DBSCAN or OPTICS). The one or more machine learning models can comprise solving, e.g., optimizing, an objective function over multiple iterations based on a training data set. The iterative solving approach can be used even when the machine learning model comprises a model for which there exists a closed-form solution (e.g., linear regression).

In some instances, the machine learning models can comprise artificial neural networks (ANNs), e.g., deep learning models. For example, the one or more machine learning models/algorithms used for implementing the disclosed methods may include an ANN which can comprise any of a variety of computational motifs/architectures known to those of skill in the art, including, but not limited to, feedforward connections (e.g., skip connections), recurrent connections, fully connected layers, convolutional layers, and/or pooling functions (e.g., attention, including self-attention). The artificial neural networks can comprise differentiable non-linear functions trained by backpropagation.

Artificial neural networks, e.g., deep learning models, generally comprise an interconnected group of nodes organized into multiple layers of nodes. For example, the ANN architecture may comprise at least an input layer, one or more hidden layers (i.e., intermediate layers), and an output layer. The ANN or deep learning model may comprise any total number of layers (e.g., 3, 4, 5, 6, 7, 8, 9, 10, 15, 20, or more than 20 layers in total), and any number of hidden layers (e.g., 1, 2, 3, 4, 5, 6, 7, 8, 9, 10, 15, 20, or more than 20 hidden layers), where the hidden layers function as trainable feature extractors that allow mapping of a set of input data to a preferred output value or set of output values. Each layer of the neural network comprises a plurality of nodes (e.g., at least 10, 25, 50, 75 100, 200, 300, 400, 500, 600, 700, 800, 900, 1000, 2000, 3000, 4000, 5000, 6000, 7000, 8000, 9000, 10,000, or more than 10,000 nodes). A node receives input data (e.g., genomic feature data (such as variant sequence data, methylation status data, etc.), non-genomic feature data (e.g., digital pathology image feature data), or other types of input data (e.g., patient-specific clinical data)) that comes either directly from one or more input data nodes or from the output of one or more nodes in previous layers, and performs a specific operation, e.g., a summation operation. In some cases, a connection from an input to a node is associated with a weight (or weighting factor). In some cases, the node may, for example, sum up the products of all pairs of inputs, Xᵢ, and their associated weights, Wᵢ. In some cases, the weighted sum is offset with a bias, b. In some cases, the output of a node may be gated using a threshold or activation function, f, where f may be a linear or non-linear function. The activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network (or other machine learning architecture), can be “taught” or “learned” in a training phase using one or more sets of training data (e.g., 1, 2, 3, 4, 5, or more than 5 sets of training data) and a specified training approach configured to solve, e.g., minimize, a loss function. For example, the adjustable parameters for an ANN (e.g., deep learning model) may be determined based on input data from a training data set using an iterative solver (such as a gradient-based method, e.g., backpropagation), so that the output value(s) that the ANN computes (e.g., a classification of a sample or a prediction of a disease outcome) are consistent with the examples included in the training data set. The training of the model (i.e., determination of the adjustable parameters of the model using an iterative solver) may or may not be performed using the same hardware as that used for deployment of the trained model.

In some instances, the disclosed methods may comprise retraining any of the machine learning models (e.g., iteratively retraining a previously trained model using one or more training data sets that differ from those used to train the model initially). In some instances, retraining the machine learning model may comprise using a continuous, e.g., online, machine learning model, i.e., where the model is periodically or continuously updated or retrained based on new training data. The new training data may be provided by, e.g., a single deployed local operational system, a plurality of deployed local operational systems, or a plurality of deployed, geographically distributed operational systems. In some instances, the disclosed methods may employ, for example, pre-trained ANNs, and the pre-trained ANNs can be fine-tuned according to an additional dataset that is inputted into the pre-trained ANN.

### BCI Software

Embodiments of the present disclosure comprise a comprehensive software framework designed to support the macroscale BCI device. The software framework in conjunction with the device can provide an integrated framework for data ingestion, device control, stimulus presentation, and data storage in standardized formats. A standardized software framework can ensure that multiple clinical and research protocols can be deployed to a patient, thereby accelerating scientific discovery and development of novel clinical applications.

In one or more embodiments, the components of this system can include:

### Device Control and Configuration

The software framework can provide users with the ability to control and configure the BCI device to meet specific clinical or research objectives. This may include customizing device settings, such as sampling rate, resolution, and anatomical targets, and defining and implementing specific stimulation paradigms or experimental protocols, if applicable.

### Stimulus Presentation and Interaction

The system can support integration with clients for presenting visual, auditory, or other stimuli in response to the BCI data or as part of a predefined experimental protocol. This functionality can involve developing APIs or plugins to interface with popular stimulus presentation software or custom-built display clients, or designing real-time data processing pipelines to extract relevant features or patterns from the BCI data that can be used to trigger or modulate stimulus presentation.

### Data Ingestion and Management

The software framework can acquire brain data and associated metadata during operation from the BCI device in real time. The data ingestion process can involve implementing communication protocols for interfacing with the BCI device and sources of metadata, such as Bluetooth, USB, Wi-Fi, or DAQ; developing efficient buffering and streaming mechanisms to handle large volumes of continuous, high-resolution brain and metadata with minimal latency; and/or implementing synchronization mechanisms to ensure accurate timing between brain and metadata.

### Data Storage and Standardization

The software framework can store the collected brain data, metadata, and related information in standardized formats for easy retrieval, sharing, and analysis. The data storage process can entail: defining standardized file formats, data structures, and naming conventions to ensure data consistency and compatibility across different users, experiments, or analysis tools; developing data export and conversion tools to facilitate data sharing with external platforms, databases, or data analysis software; and/or implementing data upload and archiving mechanisms to ensure data integrity and availability.

### User Interface and Workflow

The system can feature a user-friendly graphical user interface (GUI) to enable researchers and clinicians to easily interact with the BCI device, configure settings, monitor data acquisition, and control stimulus presentation. The user interface can provide intuitive controls, menus, and visualizations for device configuration, data monitoring, and experimental setup.

### Security and Compliance

The software framework can adhere to relevant data privacy, security, and regulatory requirements to ensure protection of sensitive patient information and compliance with ethical guidelines. Such compliance can comprise: ensuring that the software framework meets the requirements of relevant industry standards and regulations, such as HIPAA or GDPR; incorporating mechanisms for data anonymization or de-identification to protect personal health information; and/or implementing data encryption, access controls, and user authentication mechanisms to safeguard data storage, transmission, and processing.

The standardized software framework can further comprise additional analyses tools, such as, but not limited to:

### Pre-Processing Tools

These tools can comprise: motion correction algorithms for adjusting images, to compensate for patient movement during the data acquisition process; temporal filtering for applying frequency-based filters to remove non-physiological noise and retain functional signal fluctuations; spatial normalization to enable the alignment of functional ultrasound images to a standardized anatomical space for group comparisons; image segmentation algorithms comprising of both automatic and manual methods to delineate regions of interest (ROIs) in the ultrasound images; and/or algorithms for registering the ultrasound data to an anatomical MRI, which can comprise the use of sophisticated registration algorithms to align functional ultrasound data to corresponding anatomical MRI scans to assist with visualization and alignment with standardized atlases.

### Analysis Tools

These tools can comprise general linear models (GLMs), which can comprise a statistical framework for modelling the observed data as a linear combination of multiple predictors, including experimental tasks or conditions and nuisance covariates; multi-voxel pattern analysis (MBPS), which can comprise advanced methods to identify patterns across multiple voxels (rather than individual voxels in isolation) that are associated with different states or conditions, and can support importance maps and Searchlight analysis for localization of function; and/or time-course extraction algorithms, which can comprise tools to derive the signal time-course from specific ROIs or voxel clusters for further inspection or secondary analysis.

### Visualization Tools

These tools can comprise: orthogonal viewing tools for inspecting ultrasound data from different perspectives (axial, coronal, and/or sagittal) to aid in spatial understanding; 3D viewing tools for inspecting ultrasound data overlaid on reconstructed meshes of brain anatomy; time-series plotting tools for visualizing the temporal evolution of signals from selected ROIs or voxels; statistical map generation tools for visualizing 2D or 3D maps, indicating the statistical significance of the results from various analysis methods; and/or tools for visualization registration results, such as tools for visualizing 2D or 3D maps indicating the statistical significance of the results from various analysis methods.

### Focal Target Specification Tools

These tools can comprise: visualization-guided targeting for developing interactive 3D visualization tools, which can allow users to manually specify focal targets within the context of anatomical scans or atlas templates; and/or parametric map-based targeting, which can provide the functionality to define focal targets based on the results of statistical parametric maps. This can be useful when the targets are defined based on the results of functional analyses (e.g., brain regions showing significant activation or connectivity changes).

### Neuromodulation Parameter Adjustment Tools

These tools can comprise: parameter tuning interfaces, which can allow users to manually adjust neuromodulation parameters, such as the ultrasound frequency, intensity, duty cycle, phase, and visualization feedback can be provided to help users understand the spatial extent and intensity of the resulting neuromodulation; optimization algorithms for automatically adjusting the neuromodulation parameters to maximize target response, based on pre-defined objective functions. The neuromodulation parameter adjustment software tools can include software tools tailored to a specific modality of neuromodulation. For example, in the case that the modality of neuromodulation is based on ultrasound physics, the software tools can provide parameter adjustment for ultrasound technology-specific parameters, such as ultrasound frequency, intensity, duty cycle, and phase. Graphics and/or a graphical user interface can display a hypothesized or simulated delivery, e.g., direction and strength of delivery, of the neuromodulation, to the subject, optionally while considering the biomechanical constraints of the subject, such as craniometric features. The neuromodulation parameter adjustment tools can also be used for adjusting parameters for alternative neuromodulation modalities, such as, electrophysiological methods, which can include deep brain stimulation (DBS), transcranial magnetic stimulation (TMS), repetitive TMS (rTMS), vagus nerve stimulation (VNS), transcranial direct current stimulation (tDCS), electrocorticography (ECOG), or any combination thereof. As an example, a setting in the neuromodulation parameter adjustment software can be used to select a specific method of neuromodulation, which can result in showing the user a submenu of parameter settings that corresponds to the specific method of neuromodulation.

### Safety Monitoring Tools

These tools can comprise: safety limits, which can enforce the safety limits for ultrasound neuromodulation parameters (e.g., maximum intensity, total ultrasound energy) to prevent potential tissue damage; and/or warning and emergency stop, which can include warning signals when the parameters approach the safety limits, and an easy-to-access emergency stop function.

### Integration and Usability Tools

These tools can comprise: a scripting interface, such as a command-line interface for automating data processing and analysis tasks; a GUI, which is an intuitive graphical user interface for interactive data exploration and pipeline configuration; interoperability tools, which can ensure compatibility with standard data formats and integration with existing neuroimaging software; documentation and tutorials, which can include comprehensive user guides and tutorials for different user levels, from beginners to advanced users; and/or open-source and community-driven tools, which can promote an open development culture where users can contribute, share experiences, and improve the software together.

### Digital Diagnostic and Therapeutic Applications

The methods and systems disclosed herein can be used for downstream applications, such as digital diagnostics and therapies. The digital diagnostics and therapies can each comprise a separate software-as-a-medical device (SAMD), with a regulatory pathway that is independent of the macroscale BCI on which the software is supported. Development of SAMDs or neurological applications (nApps) can be used to derive further analyses and information that can be useful for clinical applications.

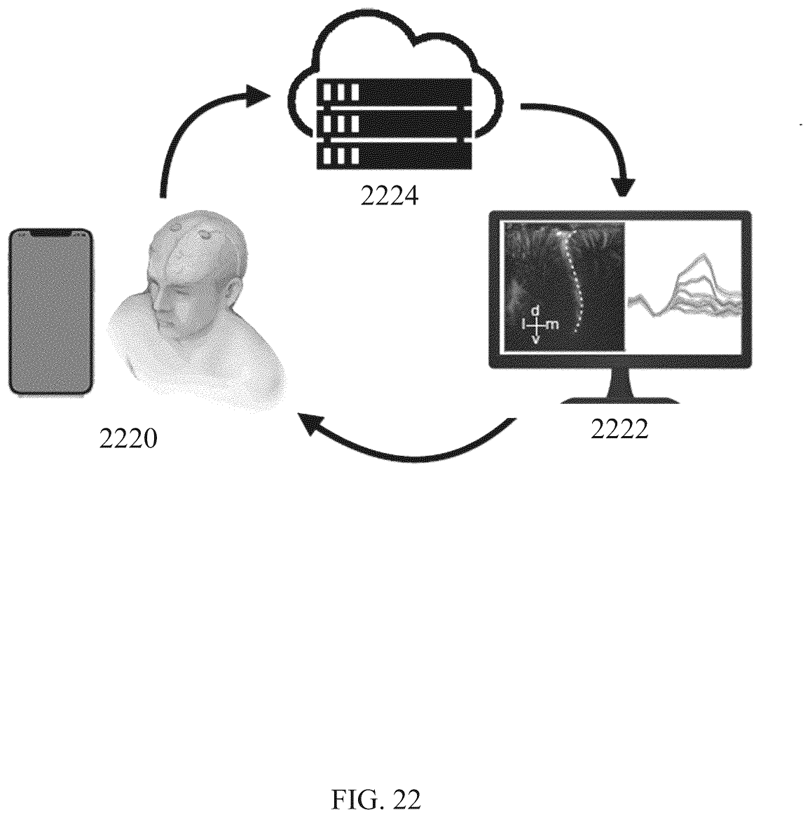

FIG. 22 illustrates an exemplary diagram of a system that includes a patient 2020 using an application that communicate with the macroscale BCI system, where the system can send data to one or more external devices 2024 (e.g., external hubs, servers, mobile devices) that may be used by one or more developers or researchers 2022 to update an existing application and/or provide new applications (e.g., nApps) for use by the patient. The data can be collected from the system by an external hub and fed into the patient application (e.g., nApp). The data can also be sent to the cloud for remote patient data access by clinicians or researchers who are able to provide patient monitoring, or new or updated patient treatments.

The nApps can be used in digital pharmacy applications. For example, when a clinician treats a patient and wishes to prescribe an nApp, they can provide an order through their (electronic health record) EHR system. The EHR system may then procure payor approval for the nApp, and deploy the nApp to the patient's implanted macroscale BCI. The clinician may be able to set and assign patient specific parameters, and data collected by the macroscale BCI, both with respect to provision of the nApp and creation of any related brain recordings, may be recorded to a cloud-based data lake and also placed into the patient's EHR. If the patient consents, the data may be de-identified and made available to support further research and development. Payment for the nApp prescription may also flow from the Payor (and also applicable co-pays) to the developer of the nApp. The digital pharmacy may be intended to provide the same functionalities for nApps as are made by today's pharmacies with respect to existing medications.

While implantation of the macroscale BCI and prescription of an nApp can use a clinical basis, once implanted, it is feasible to provide wellness and even non-health related applications to patients. This functionality largely emerges from the ability of the macroscale BCI to interface with large volumes of the brain, hence extending beyond the circuits directly tied to the clinical indication that justified implantation. Indeed, provided that such applications are focused on monitoring, rather than stimulation, it is feasible for the methods and systems disclosed herein to entail a set of functions that are “automatically safe” and the macroscale BCI can then function as a non-clinical interface between the patient and any number of applications. Subsequent to digital pharmacy applications, a separate application marketplace can be provided to create access to data related to the methods and systems disclosed herein.

The data deriving from the methods and systems disclosed herein can also be related to a shared registry. The registry can allow for developers to quickly locate existing patients, and offer to enroll them into new studies, based on the inclusion and exclusion of new studies. In some instances, the creation of such a registry can comprise clinical sites to offer to subjects working with the systems and methods discussed herein to join the registry. The subjects can opt to be included in or excluded from the registry.

In one or more embodiments, an nApp in accordance with embodiments of this disclosure may target one or more use cases summarized in Table 1 provided below:

TABLE 1

| Use case | Indication | Setting |
|---|---|---|
| Intraop tumor identification | Tumor | OR |
| Tumor recurrence monitoring | Tumor | At home |
| Tumor - targeted drug delivery | Tumor | At home |
| OCD biomarkers | OCD, Psych | Monitoring unit |
| OCD therapy | OCD, Psych | At home |
| Pain biomarkers | Pain, Psych | Monitoring unit |
| Pain therapy | Pain, Psych | At home |
| Depression biomarker | Depression, Psych | Monitoring unit |
| Depression therapy | Depression, Psych | At home |
| Replacement for Intracranial | TBI, intracranial/ventricular | ICU, OR |
| Pressure | hemorrhage, subarachnoid | |
| | hemorrhage | |
| Vascular dementia | | |
| Moyamoya | | |
| Subarchnoid hemorage - | Stroke | Monitoring unit, OR |
| Monitoring | | |
| Orthostatic hypotension | | |
| Migraines - therapy | | |
| Epileptogenic zone mapping | Epilepsy | Monitoring unit, OR |
| Functional mapping - imaging | Epilepsy, Tumor | Monitoring unit, OR |
| Epileptogenic zone causal | Epilepsy | Monitoring unit, OR |
| mapping | | |
| Functional mapping - causal | Epilepsy, Tumor | Monitoring unit, OR |
| Parkinson's Disease - motor | Parkinson's Disease | At home, Monitoring unit |
| therapy | | |
| Parkinson's Disease - | Depression, Parkinson's | At home, Monitoring unit |
| depression therapy | Disease, Psych | |
| Parkinson's Disease - | Cognitive Parkinson's Disease | At home, Monitoring unit |
| Cognitive therapy | | |
| Parkinson's Disease - | Essential Tremor | At home, Monitoring unit |
| Cognitive biomarker | | |
| Essential Tremor - Motor | Essential Tremor | At home, Monitoring unit |
| therapy | | |
| Essential tremor - alcoholism | Essential Tremor | At home, Monitoring unit |
| biomarker | | |
| Essential tremor - alcoholism | Eating disorder, Psych | At home, Monitoring unit |
| therapy | | |
| Eating disorder | Bipolar, Psych | At home, Monitoring unit |
| Bipolar biomarker | Bipolar, Psych | At home, Monitoring unit |
| Bipolar therapy | TBI, subarachnoid hemorrhage | At home, Monitoring unit |
| Cortical Spreading depression - | ALS | ICU |
| monitoring | | |
| ALS monitoring | Stroke, subarachnoid | At home |
| | hemorrhage | |
| Vasospasm monitoring | | ICU |
| Dystonia - therapy | Cognitive | At home, Monitoring unit |
| Memory - therapy | | At home, Monitoring unit |
| enhancement | | |
| Acute first responded | Stroke, TBI | Noninvasive |
| diagnostics | | |
| Hydrocephalus monitoring | | ICU |
| Trauma prognosis | Stroke, TBI | ICU |
| Drug discovery | Many | preclinical |
| Visual prosthesis | Blindness | At home |
| Intraoperative registration | General surgical | OR |
| Intraoperative stroke | Stroke | OR |
| monitoring | | |
| Understanding and facilitating | Scientific | Monitoring unit |
| meditation states | | |
| Substance abuse disorder - | Substance abuse | At home, Monitoring unit |
| biomarker | | |
| Substance abuse disorder - | Substance abuse | At home, Monitoring unit |
| therapy | | |
| Functional localization - pre- | Scientific | Preclinical |
| clinical | | |
| BCI | | At home, ICU, Monitoring |
| | | unit, OR, preclinical |
| Schizophrenia - Hallucinations | | At home, Monitoring unit |
| Working memory | Cognitive, Memory | At home, Monitoring unit |

### Computer Systems and Networks

FIG. 24 illustrates an example of a computing device or system in accordance with one embodiment. Device 2400 can be a host computer connected to a network. Device 2400 can be a client computer or a server. As shown in FIG. 24, device 2400 can be any suitable type of microprocessor-based device, such as a personal computer, workstation, server or handheld computing device (portable electronic device) such as a phone or tablet. The device can include, for example, one or more processor(s) 2410, input devices 2420, output devices 2430, memory or storage devices 2440, and communication devices 2460. Software 2450 residing in memory or storage device 2440 may comprise, e.g., an operating system as well as software for executing the methods described herein. Input device 2420 and output device 2430 can generally correspond to those described herein, and can either be connectable or integrated with the computer.

Input device 2420 can be any suitable device that provides input, such as a touch screen, keyboard or keypad, mouse, or voice-recognition device. Output device 2430 can be any suitable device that provides output, such as a touch screen, haptics device, or speaker.

Storage 2440 can be any suitable device that provides storage (e.g., an electrical, magnetic or optical memory including a RAM (volatile and non-volatile), cache, hard drive, or removable storage disk). Communication device 2460 can include any suitable device capable of transmitting and receiving signals over a network, such as a network interface chip or device. The components of the computer can be connected in any suitable manner, such as via a wired media (e.g., a physical system bus 2480, Ethernet connection, or any other wire transfer technology) or wirelessly (e.g., Bluetooth®, Wi-Fi®, or any other wireless technology).

Software module 2450, which can be stored as executable instructions in storage 2440 and executed by processor(s) 2410, can include, for example, an operating system and/or the processes that embody the functionality of the methods of the present disclosure (e.g., as embodied in the devices as described herein).

Software module 2450 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 2470, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art may appreciate that the above processes may be routines or modules within other processes.

Software module 2450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

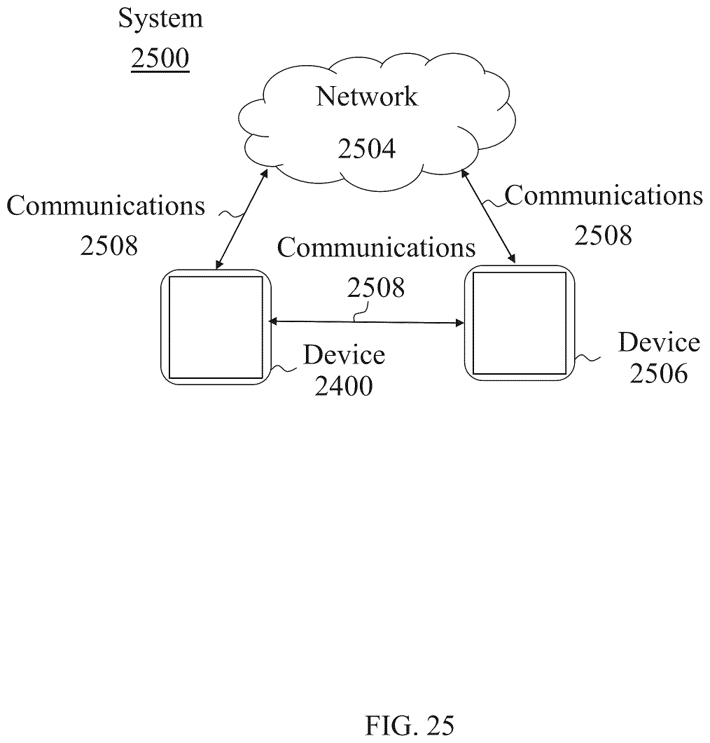

Device 2400 may be connected to a network (e.g., network 2504, as shown in FIG. 25 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 2400 can be implemented using any operating system, e.g., an operating system suitable for operating on the network. Software module 2450 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 2410.

FIG. 25 illustrates an example of a computing system in accordance with one embodiment. In system 2500, device 2500 (e.g., as described above and illustrated in FIG. 25) is connected to network 2504, which is also connected to device 2506. In some embodiments, device 2506 is a peripheral control unit capable of controlling at least one ultrasound transducer.

Devices 2400 and 2506 may communicate, e.g., using suitable communication interfaces via network 2504, such as a local area network (LAN), virtual private network (VPN), or the Internet. In some embodiments, network 2504 can be, for example, the Internet, an intranet, a virtual private network, a cloud network, a wired network, or a wireless network. Devices 2400 and 2506 may communicate, in part or in whole, via wireless or hardwired communications, such as Ethernet, IEEE 802.11b wireless, or the like. Additionally, devices 2400 and 2506 may communicate, e.g., using suitable communication interfaces, via a second network, such as a mobile/cellular network. Communication between devices 2400 and 2506 may further include or communicate with various servers such as a mail server, mobile server, media server, telephone server, and the like. In some embodiments, Devices 2400 and 2506 can communicate directly (instead of, or in addition to, communicating via network 2504), e.g., via wireless or hardwired communications, such as Ethernet, IEEE 802.11b wireless, or the like. In some embodiments, devices 2400 and 2506 communicate via communications 2508, which can be a direct connection or can occur via a network (e.g., network 2504).

One or all of devices 2400 and 2506 generally include logic (e.g., http web server logic) or are programmed to format data, accessed from local or remote databases or other sources of data and content, for providing and/or receiving information via network 2504 according to various examples described herein.

### Examples

Examples 1-3 described herein leverage a simulation framework for rapid prototyping of imaging and neuromodulatory solutions. The simulation approach was based on maximizing the physical, anatomical, and physiological realism of the system. It generated ultrasound images based on first principles of wave propagation, reflection, aberration, reverberation, and scattering within soft tissue and vasculature. These methods were based on numerical solution tools that solve the full wave equation. The physics-based approach presented in the examples allowed for the rapid design iterations of the 1) transducer array, 2) imaging sequencing, and 3) optimizing imaging parameters. Simulations of acoustical propagation within the brain volume were performed to determine the total illumination capabilities of the arrays. The array configurations were then iteratively simulated, including the array properties (e.g., placement geometry, and frequency) and tissue properties (e.g., scatterers contrast, clutter, target depth, neurovascular flow characteristics) to optimize imaging processes. The otherwise vast parameter space was limited by implementing use-case specific constraints. For example, simulations were limited to physically achievable locations for transducer placement, by using physiological data extracted from human CT/MRI scans. In addition, the imaging fields could be constrained by using brain regions of interested associated with a particular neurological pathology.

### Example 1—Indicative Thermal Budget

Thermal load can depend on specific imaging or neuromodulatory sequence parameters, including the number of transmits, duration, and duty cycle. These parameters can be optimized in conjunction with hardware development. A thermal budget on power consumption can be estimated by solving Penne's equation for bio-heat transfer using numerical simulation tools for given hardware form factors. Power consumption is split among components implanted in the head and in the chest. Estimates for electrical power consumption using conversion efficiency estimates for CMUT arrays, per-element power consumption of the analog front-end and digitization, digital compute cost of filtering, demodulation, beamforming, and power-doppler estimation, and other chip processes (e.g., power management, timing circuitry, etc.). The various imaging and neuromodulatory sequence parameters that affect heat generation can be optimized by maintaining a maximum temperature elevation within accepted standards.

During operation of the macroscopic BCI system, one or more components, e.g., the peripheral controller and the implantable transducers, may undergo an increase in temperature. For instance, as the implantable transducer is emitting and/or receiving ultrasound waves, the implantable transducer is expected to experience a rise in temperature. A dominant constraint that shapes the imaging solution is the power budget that the disclosed systems can use, while conforming to the thermal safety requirements of an implanted system. Namely the systems described herein should not heat the subject's tissue beyond 2 degrees C., with an absolute limit of 39 degrees C. for the brain (ISO 14708-3). Computational studies can provide accurate preliminary estimates for modeling thermal characteristics of cranial implants (e.g., implantable transducers) in accordance with embodiments of this disclosure.

FIG. 26 depicts non-limiting exemplary results from numerical simulations for an example cranial implant 2612, provided a thermal power budget of ˜350 mW, while conforming to ISO safety standards of tissue heating. Formation of a single 2D functional brain image per second can consume approximately 20 mW of power within the cranial implant 2612. Therefore, the envisioned system can support acquisition of around 16 2D slices of functional brain data per second, which can allow for a high degree of spatial coverage for our indicative imaging solution. An exemplary imaging solution can be visualized in FIG. 15B, where the 2D planes are shown in cross-section, at scale, overlaid on an anatomical MRI demonstrating significant and programmable coverage A more in-depth feasibility analysis of the components of an ultrasound imaging system, in accordance with the embodiments disclosed herein, was performed. Namely, the cranial implant 2612 and the remote processing unit of the ultrasound imaging system were analyzed.

This example depicts results from a numerical model of the implantable transducer's thermal impact on neighboring tissues. The estimated power budget is subjected to FDA safety constraints using simulation software (e.g., COMSOL). The results are visualized in FIG. 26 (depicting scalp 2614, brain 2616, and subrachnoid space 2618). In some aspects, the numerical model assumes a three-dimensional 25 mm diameter cylindrical model of the cranial implant 2612, extending through the depth of the skull 2610, as depicted in FIG. 2B and FIG. 26. The implant extends to 35 mm on the surface of the skull 2610 to provide a physical substrate to secure the implant to the skull as well as to dissipate additional heat. The components of the physical model were defined using validated model parameters, as depicted in Table 3 and Table 4. Numerical simulations resulted in a steady-state thermal budget of 350-400 mW, as indicated in FIG. 26.

TABLE 3
Model parameters

| Tissue Layer | Thickness (mm) | SU | Thickness (mm) |
|---|---|---|---|
| Scalp 2614 | 3.5 | Ti Shell | 1 |
| Skull 2610 | 7 | Ti Flange | 2 |
| Brain 2616 | 50 | PCB | 1 |
| Subarachnoid Space | 3.2 | Air gaps | 1.5 |
| 2618 | | | |

TABLE 4
Model parameters

| | k | ρ_{b}C_{b}ω | Qₘ |
|---|---|---|---|
| Tissue Layer | (W/(m K)) | (W/(m³ K)) | (W/m³) |
| Scalp 2614 | 0.342 | 8954.9 | 1100 |
| Skull 2610 | 0.650 | 4029.7 | 26 |
| Brain 2616 | 0.528 | 39483 | 10383 |
| Subarachnoid Space | 0.570 | — | — |
| 2618 | | | |

These results can provide an upper bound on the expected power budget for each implantable transducer. The use of additional power would heat the implantable transducer beyond what can be considered safe under current guidelines.

### Example 2—Parameter Optimization for Imaging with Ultrasound Transducers

According to embodiments of the present disclosure, the BCI system can include multiple implantable transducers. In such embodiments, placement of the implantable transducers on the skull of a subject can impact the efficacy of the imaging and neuromodulation functionalities of the system. Optimizing placement of the implantable transducers can produce constructive or destructive interference. In order to efficiently deliver ultrasound energy to the desired regions of the brain, it is useful to determine optimal placement of the implantable transducers on the skull. A simulation for determining a potential multi-implantable transducer design is detailed below.

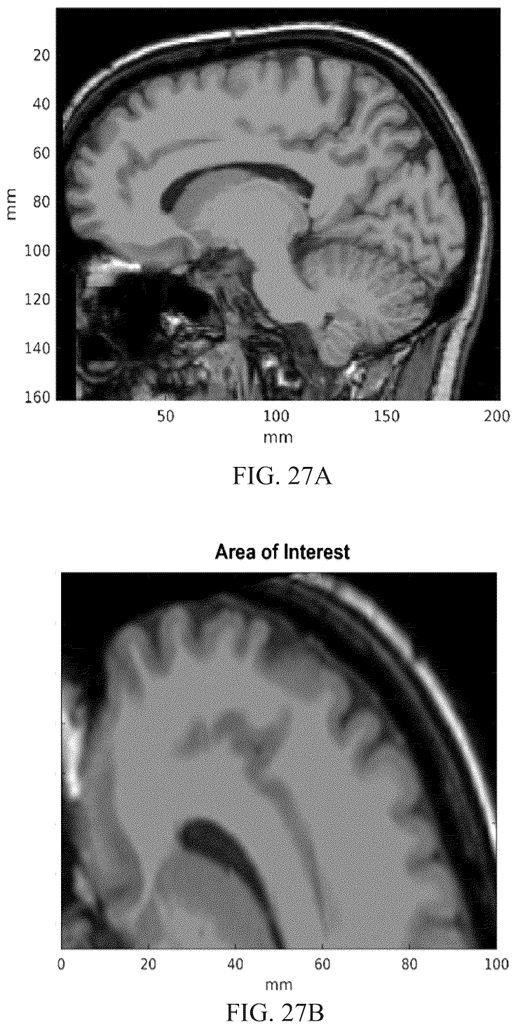

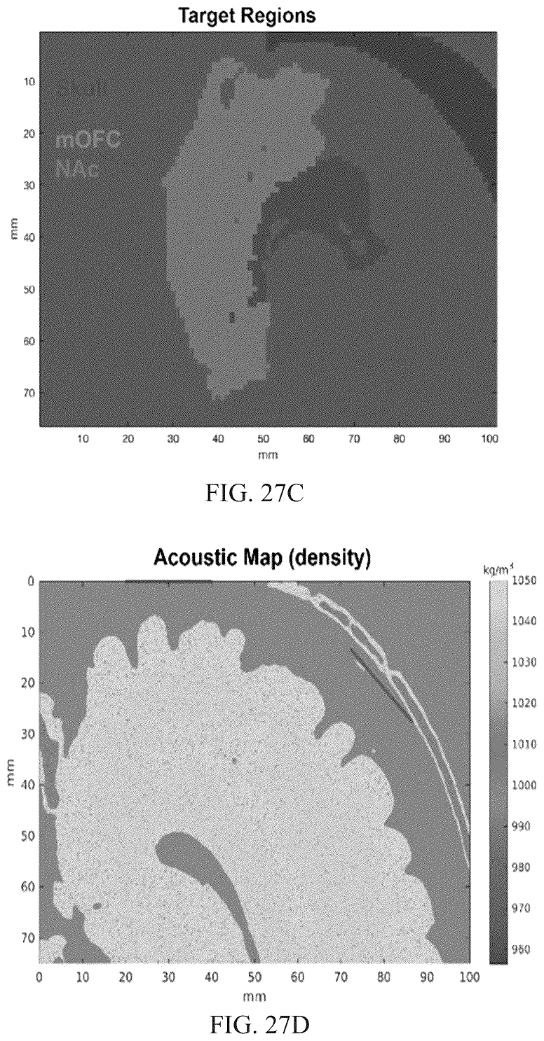

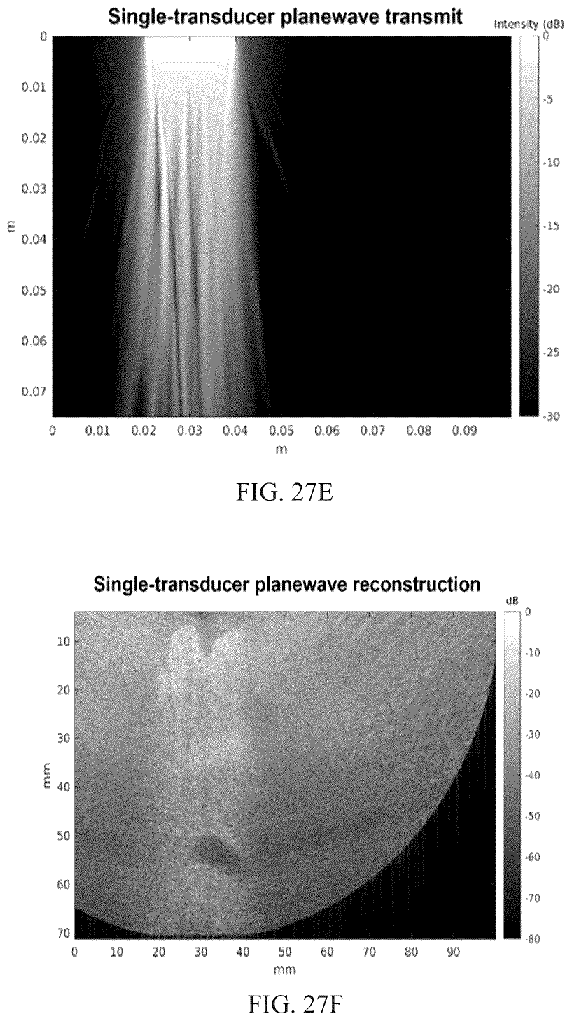

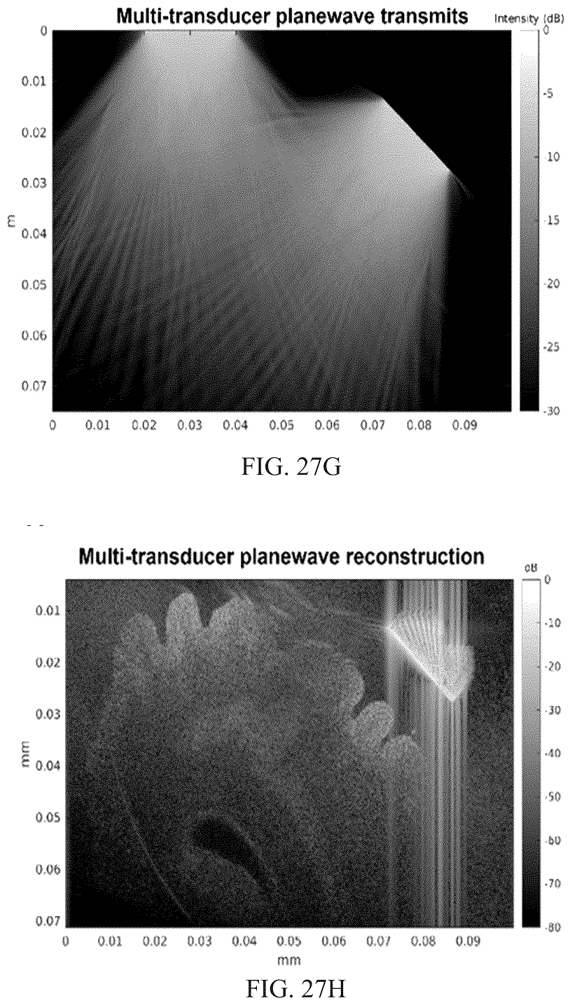

Sequence design and validation approaches can be explored via the simulation of imaging and neuromodulatory pulses for multi-array design. The simulation of imaging and neuromodulatory pulses assume either (i) independent or (ii) “pitch-catch” planewaves compounding in anatomically and acoustically calibrated simulations. The results are based on two virtual transducers implanted at 45 degrees from each other with a 20 mm aperture and a λ/2 pitch. Other configurations in placements, array size, pitch, kerf, frequency, number of arrays are possible. Magnetic resonance (MR) scans of one or more human subjects were used (FIG. 27A) to determine the general target area (FIG. 27B) and location of specific circuits (FIG. 27C) (here the Nucleus accumbens, anterior cingulate, and orbitofrontal cortex, regions relevant to circuit-based mechanisms of depression). The scanning data were then converted into maps of the acoustical properties (FIG. 27D), including speed of sound, density, attenuation, nonlinearity, and scatterer brightness. Plane wave transmits were propagated (5 MHz, 3 cycles, delta theta=4) into the acoustical field. Reflections generated an ultrasound image based on the first principles of propagation using a conventional delay-and-sum compounding beamformer (FIG. 27F, FIG. 27H) as well as the intensity field based on the heterogenous propagation path in brain tissue (FIG. 27E, FIG. 27G).

Two approaches to imaging are presented that illustrate differences in image quality based on imaging parameters: (i) single plane waves emitted by a single array (FIG. 27F, FIG. 27G) and (ii) 23 compounded plane waves emitted by two arrays oriented at 45 degrees from each other (FIG. 27H, FIG. 27I). Additional quantification of resolution and contrast were provided by matched simulations of an ATS small parts phantom for the two scenarios (FIG. 27J, FIG. 25K). The phantom can provide an ideal imaging scenario because this homogenous material may not be subject to the same sources of image degradation that can be encountered in the brain.

Covering the brain volume using small implantable ultrasound devices that can fit within burr-hole surgeries can be solved by designing arrays and ultrasound beams that can be easily steered to large angles in azimuth and elevation. This capability is inversely proportional to the element size or pitch with improvements plateauing at half the wavelength of the transmit frequency

$$( \frac{λ}{2} ) ,$$

which is commonly used in sector-scan imaging. Even though PZT λ/2 linear arrays, which have large rectangular elements, have been used extensively in clinical applications for over two decades, their use in matrix imaging has been restricted to a handful of research implementations due to manufacturing complexity. In some aspects, reliable CMUT fabrication technologies may be used that simultaneously solves the array dicing, sensitivity, and electronic interconnect problems, that fully addressable λ/2 pitch matrix arrays. Furthermore, in some aspects, large angular sensitivity can be even more apparent in volumetric imaging: for example, a single ultrasound array with a 16.2 mm base imaging to 80 mm depth with a 45-degree angular sensitivity may have an imaging volume of 315 cc, which is ˜25% of the average volume of a human brain.

The spatial resolution limits of a single array system can be relatively straightforward to determine. The resolution and sensitivity to fUS signals using a multi-array imaging system, however, is complex, and depends on a broad array of parameters, including hardware capabilities (e.g., noise levels in amplifiers, digitization bit depth), choice of transmit sequence (e.g., plane wave, focused imaging, synthetic aperture approaches), frame-rate or ensemble lengths, and post processing algorithms (e.g., beamforming, single value decomposition (SVD) filters), the volume of brain being imaged for a task (e.g., whole brain vs. targeted), and the temporal resolution at which the fUS signal is obtained (continuous monitoring vs. on-demand). Furthermore, these choices and the tradeoffs they represent can have a complex dependency on power consumption and heating which can be strictly limited to avoid thermal bio-effects.

### Example 3—Feasibility Analysis for Imaging Nervous Systems

The systems and methods described herein can generate high-quality functional images that attain large coverage and high framerate within the constraints of an implantable form-factor. The disclosed systems and methods can comprise optimized methods of transmit and receive techniques, as well as for subsequent processing for low-power applications. The disclosed systems and methods can support multiple imaging modes, optimized for different use-cases, as determined by requirements such as imaging depth, coverage, and framerate.

Different sequences for functional imaging can include wide beam/explososcan, row-column addressing, sub-aperture plane-wave compounding, and sparse aperture imaging approaches. These methods have trade-offs in terms of imaging depths, framerate, compounding effectiveness, and sensitivity to heterogeneity and thus can have varied relevance depending on the specifics of use case requirements. The simulations described herein can produce a first-order characterization of the image sequence design, including point spread function analysis, contrast to noise ratio, electronic and thermal noise, grating lobes, and their dependence on frequency. The models can be extended to include the effects of brain heterogeneity, off-axis and reverberation clutter, blood pool contrast, neurovascular flow differentials (modeled and simulated within the validation framework above), should the models prove helpful in improving the generalization accuracy from the modeling to in vivo work. The effects of compounding, frame rates, noise sensitivity, and array design can be included in the analysis. Receive beamforming approaches, apodizations, effects of beamforming operations, compounding, partial compounding, coherence factors, and muxing may be tested with an understanding that solutions may be implemented in hardware and thus include constraints like the number of channels that can be digitized, communication bandwidth between the cranial implant and remote processing module, storage and compute constraints in digital processing, among others. Therefore, imaging design can be done as part of a co-optimization with hardware. The optimization parameters can include estimating the total number of frames required to populate a brain volume, the size/locations of the beamforming grid, RF vs. IQ processing, the number of channels required, and the complexity of operations within the signal processing chain, and how these various processing shapes are split between the different hardware modules that comprise the system, e.g., within the cranial implant, the chest implant, or on a remote externalized device.

In some embodiments, power-consumption feasibility calculations based on 2D plane-wave imaging were used as a foundational baseline, for an indicative transmit, receive, and processing approach to characterize power requirements and resulting functionality. In this instance, functional brain data can be acquired slice-by-slice, programmably specified by the subset and timing of which transducer elements can be activated on the 2D transducer matrix, as shown in FIG. 15A. This slice-by-slice approach can be thought of as being similar to how volumetric imaging can be performed in functional MRI. For these indicative calculations, the methods and systems described herein assume a split-package form factor composed of a cranial implant and remote processing module that can be externalized in some implementations of the described systems and can be implanted in the chest for other implementations of the described systems. The cranial implant can comprise the sensor array, integrated analog processing and digitization, digital preprocessing, and wireline data communication. The remote processing modular can be responsible for digital processing.

Cranial implant: The cranial implant can comprise the implantable transducer array, integrated analog processing and digitization, digital preprocessing, and in some embodiments, wireline data communication. Specific variables that impact power consumption include low noise amplifier (LNA) noise, bit depth of digital conversion, the imaging sequence determining the number of elements for transmit, receive, and digitization, the number of transmissions used to estimate the power doppler estimate of cerebral blood volume, and the receive window (amongst other factors). The present example provides indicative power consumption calculations for the 2D plane wave imaging sequence, separately for each of the main processing stages. The present example assumes each functional 2D image is generated from 250 images, created from distinct transmit and receive events. This number is the minimum value that allows for accurate detection of functional brain activity using advanced processing methods that have been validated using non-human primate data.

Assuming a sub-aperture of 1,000 transducer elements on an ultrasound array is energized to create the 2D plane-wave, a capacitive load of 2 pF, an operating frequency f₀=5 MHz, and a peak-to-peak voltage of Vₚ₂ₚ=50V, the instantaneous transmit power of 12.5 W can be estimated, which is in line with current state-of-the-art systems. For imaging, this peak power is seen only during the transmit pulse, which is short (˜0.6 μs per image). Assuming 250 images are acquired, a temporal-average power of Pₜᵣₐₙₛₘᵢₜ=1.875 mW is estimated. To estimate transmit power consumption, P=0.5×C×V²×f is used as an approximation to estimate power consumed in an AC circuit with a capacitive load. Here, C represents the capacitance, V represents the peak voltage swing, and f represents the signal frequency. P is derived from the expression for energy stored in a capacitor E=0.5×C×V², and then taking into account the energy change over time in the AC signal by multiplying with the frequency f.

For an imaging depth of 8 cm, speed of sound of 1540 m/s, AFE and digitization power consumption of approximately 0.66 mW/element (when operating continuously), with ˜1000 receive elements and microbeamforming down to ˜128 channels prior to digitization, the average power consumption of the receive chain is estimated to be P_{receive}=17 mW. The power consumption of an analog front-end (AFE) and digitization receive chain for ultrasound imaging can be estimated based on element-level power consumption, which for integrated designs is approximately 0.5 mW-1 mw per element to get to 10-bit digital data. 1000 elements are assumed to be active to receive the back-scattered echoes, and these elements are summed along the elevational axis in the analog domain to reduce channel count to 128. An indicative per-element power consumption of 0.66 mW is selected. Energy consumption can be calculated based on the time of flight of the received ultrasound signals and the total number of images:

$$Receive\_power = ( receive\_window \_per \_image ) \times AFE\_power \_consumption \times number\_of \_images$$

$$Receive\_power = ( 2 \times imaging\_distance / speed\_of \_sound ) \times ( AFE\_per \_element \_power \times number\_of \_active \_elements ) \times number\_of \_images$$

$$Receive\_power = ( 2 \times 0.08 / 1540 ) \times ( 0.66 {e}^{- 3} \times 1000 ) \times 250 = 17.14 mW$$

Once the raw data is digitized, the digitized data can be transferred directly or undergo per-channel digital processing such as IQ demodulation, smoothing, and decimation to reduce data load. For this exercise, the raw digital data is assumed to be transmitted: 128 digital channels sampled at two times the transmit frequency (e.g. 10 MHz) with a bit depth of 10. These parameters would result in 332 Mb/s of data to transfer. This rate can be computed based on the per-image data time and the total number of images. The data per image is a function of the number of channels, sampling rate, sampling window, and bit depth. Thus, the total data is given by:

$$total\_data = data\_per \_image \times number\_of \_images$$

$$total\_data = ( receive\_w indow \_per \_image \times sampling\_rate \times number\_of \_channels \times bit\_depth ) \times number\_of \_images$$

$$total\_data = ( ( 2 \times imaging\_distance / speed\_of \_sound ) \times sampling\_rate \times number\_of \_channels \times bit\_depth ) \times number\_of \_images$$

$$total\_data = ( 2 \times 0.08 / 1540 \times 10 {e}^{6} \times 128 \times 10 ) \times 250 = 332 Mb / s .$$

Channel loss for a wireline transmitter is dependent on design, materials, frequency and distance. For our data rates and relatively short distance between the cranial implant and chest unit, an energy efficiency of around 1.5 pJ/bit can be assumed to be achieved. Therefore, power consumption for a data rate of 332 Mb/s would equate roughly to P_{interconnect}=0.5 mW. Thus, if the feasibility analysis is limited to the active processes to transmit, receive, and preprocess data for a 2D planewave image, we expect a total cranial energy expenditure of:

$${P}_{Cranial Implant} = {P}_{transmit} + {P}_{receive} + {P}_{interconnect} = 1.875 + 17 + 0.5 = 19.3 \; 75 mW$$

These power consumption values would allow for the acquiring of roughly 16 2D slices of functional brain data every second, while remaining within the computed thermal budget of ˜325 mW.

Remote processing unit: The remote processing module can be responsible for digital processing of the raw ultrasound data into functional brain images. To understand power requirements, the number of operations (e.g., multiply and accumulates or MACs) required by each processing stage may be estimated, and then the power based on an indicative process (e.g., 22 nm) may be estimated. For purposes of feasibility calculations, the following three processing stages are estimated: 1) IQ demodulation of the RF data to baseband, filtering and decimation; 2) image formation (“beamforming”); and 3) power doppler estimation. Alternative processing schemes are possible, but this ordering is understood to minimize power consumption. Demodulation, filtering and decimation given our system parameters requires on the order of 400 million MACs. Regarding IQ demodulation, when analyzing ultrasound, the raw RF signals are demodulated into in-phase and quadrature (IQ) components. This typically involves multiplying the RF signal by a cosine wave (for the in-phase component) and a sine wave (for the quadrature component), each at the carrier frequency. Regarding filtering and decimation, data passes through a low-pass filter after the demodulation. The number of MACs for this operation is dependent on the number of filter taps and the number of samples. If the filter has M taps (e.g., 5 for a biquad IIR filter) and there are N samples, M×N MACs per I or Q branch are used. Hence, the total number of MACs would be 2×M×N for this stage. Decimation can involve downsampling, which does not require MAC operations (although an anti-aliasing filter may be included).

$$MACs = 2 \times M \times N \times number\_of \_images$$

$$MACs = 2 \times 5 \times 1038 \times 128 \times 256 = 340 {e}^{6}$$

This results in two multiplication operations per sample, per channel: one for the in-phase and one for the quadrature component. So, the number of MACs is 2×N×number_of_images, where N is the total number of samples per image. In our case:

$$MACs = 2 \times N \times number\_of \_images$$

$$MACs = 2 \times ( receive\_window \_per \_image \times sampling\_rate \times number\_of \_channels ) \times number\_of \_images$$

$$MACs = 2 \times 1038 \times 128 \times 2 \; 56 = 68 {e}^{6}$$

Image formation through beamforming and coherent compounding can use on the order of 8.2 billion MACs. The number of estimated required MACs justifies development of a custom DSP chip. The final stage of processing estimates power-doppler from the ensemble of collected images. In the implanted form-factor, this processing may be performed on-chip or may be opted to transfer the reconstructed B-mode images off-device for subsequent processing. For example, the B-mode ensemble for the 16 2D slices of data would be on the order of 262 Mb/s after compounding.

Regarding the feasibility analysis' incorporation of beamforming and coherent compounding, the analysis considers that in RF space, information from a single scatterer is spread across channels as determined by the time of flight from the position of the scatter in the medium to the position of the transducer, defining a hyperbolic shape in the mixed space-time space of the RF signals. Beamforming is the process of summing along this hyperbola to integrate all the energy from the scatterer that would originate from each point in physical space. In practice, values of hyperbola are determined by an interpolation which is precomputed. Thus the beamforming operation for each beamformed pixel is proportional to the number of channels and the interpolation factor. In practice, this number is reduced based on the effective f-number of the imaging setup, which takes into account that the effective signal-to-noise of the back-scattered echoes is not uniform across the hyperbolic signature. This is largely determined by the directivity of the transducer elements. Here we take a conservative estimate and assume the number of MACs per beam-formed pixel is on the order of 500. This means the total MACs are governed by the number of beam-formed pixels per image (here we assume a 256×256 image) and the number of images:

$$MACs = number\_beamformed \_pixels \times 500 \times number\_of \_images$$

$$MACs = 256 \times 256 \times 500 \times 250 = 8.2 {e}^{9}$$

### Example 4—Neuromodulation Feasibility Analysis

Neuromodulation sequence design places fewer constraints on the acoustical design of the transducer (e.g., focusing capabilities and sensitivity) and more constraints on acoustical load. The reasoning for the reduced constraints is because neuromodulation uses pulse trains lasting milliseconds, while imaging pulse trains are typically <1 us. Focal gain estimates mapped throughout the intervention volume to estimate required pressure levels at the transducer surface. The estimates can then be obtained with derated water tank measurements for upper bounds and with simulations of heterogenous tissue properties described in the imaging section, which can provide a more accurate representation effects of attenuation, aberration, non-linearity, registration error, and focal spot characteristics.

Although ultrasound imaging and neuromodulation can occur through the same hardware, the technical requirements are fundamentally different. Ultrasound imaging works by sending brief (<1 ρs) pulses of mechanical energy into the brain, measuring back-scattered echoes, and estimating the neurovascular state of the tissue. Given the extremely short pulse durations, the majority of power consumption consumed for ultrasound imaging is in the receive processing. Ultrasound neuromodulation, in contrast, often relies on the ability to deliver extended pulse trains, up to on the order of 10 ms, without the need for any receive processing, as depicted in FIG. 15C. Thus, the primary technical challenges for ultrasound stimulation relate to the ability to deliver sufficient acoustic intensity to the neuromodulation target for the extended durations that characterize effective neuromodulation parameters in the published literature. The non-limiting exemplary results assume a system comprising of three transducers, each a square grid, consisting of 112×112 elements with a pitch of 150 μm (e.g., the same arrays used for imaging). The use of multiple transducers as part of a neuromodulatory system can provide for better spatial specificity of the focal region as well as increased intensity through the constructive summation of the beam profiles produced by each array (in the case of 3 arrays, the constructive interference can allow for a nine-fold increase in intensity). FIG. 15D depicts this exemplary design concept. The results indicate that the systems described herein can deliver spatial peak, temporal average intensities (I_SPTA) of ˜69, 27, and 14 W/cm² at focal depth of 4, 6, and 8 cm, approximately double the maximum I_spta reported in the neuromodulation literature. Further, the systems described herein can also deliver spatial peak pulse average (I_sppa) of 25 W/cm² for pulse durations of 30 ms over extended periods of time at 8 cm depth, matching the peak pulse-average intensity documented in the literature.

The above feasibility analysis can be examined in further detail. The above feasibility analysis is more precisely concerned with the disclosed systems' ability to insonify a focal target location with transmit specifications validated to induce neuromodulatory effects. Parameters relevant to technical feasibility include the ability to deliver sufficient acoustic intensity (e.g., I_spta up to 7 W/cm²) and maintain elongated pulse trains (e.g., up to ˜30 ms).

The spatial-peak temporal-average intensity (I_spta) that can be delivered to focal targets at several representative depths is characterized. The non-limiting example system described herein is composed of three transducers, each a square grid, consisting of 112×112 elements with a pitch of 150 μm. The use of multiple transducers as part of a neuromodulatory system allows for improved spatial specificity of the focal region, as well as increased intensity through the constructive summation of the beam profiles produced by each array. The approach described in the present example is to compute the I_spta for each single transducer and compute the result of the full system assuming linearity.

From the simulation modeling work depicted in FIG. 26, the electrical power budget is limited to 325 mW. Taking into account the conversion efficiency from electrical to acoustic power of 70%, the resulting acoustic power (Pa) produced by the CMUT array can be calculated as:

$$P a = Efficiency \times Pe = 0.7 \times 0.3 \; 25 W = 0.2275 W$$

The total active area (A) of the CMUT array is:

$$A = {( Number of elements along one side \times pitch )}^{2} = {( 112 elements \times 150 {e}^{- 4} )}^{2} = 2.82 {cm}^{2}$$

The resulting spatial-peak temporal-average intensity at the transducer surface (Is_spta) is:

$$Is\_spta = P a / A = 0.2275 W / 2.82 {cm}^{2} = 0.0 \; 806 W / {cm}^{2}$$

The above calculations assume a typical duty cycle of ˜50%. Thus, I_spta would correspond to a I_sppa of 0.16 W/cm² and a measured surface pressure of 0.071 MPa, which is far below the technical capabilities of commercial CMUT arrays, e.g., a hypothetical standard CMUT array is able to achieve 1 MPa of surface pressure for an RF voltage drive of 50V.

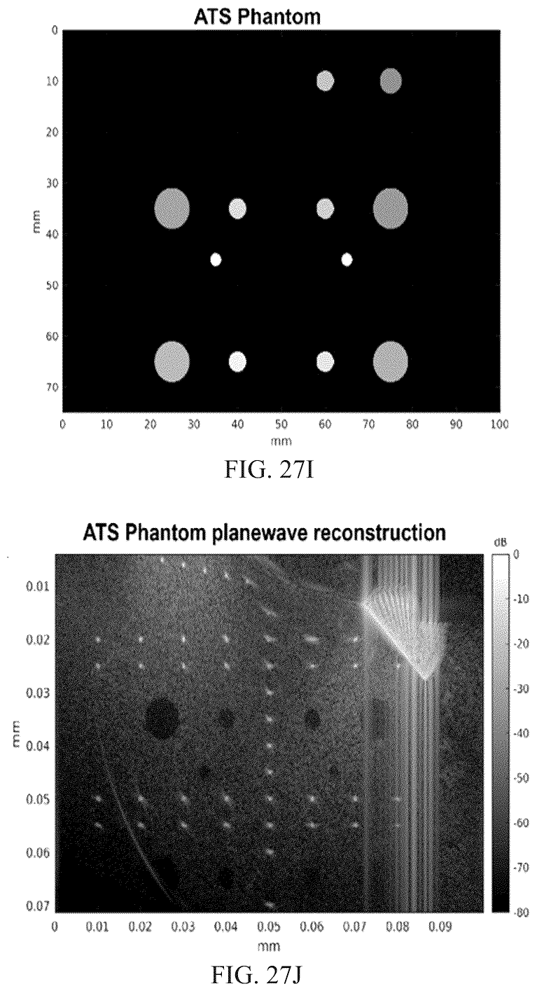

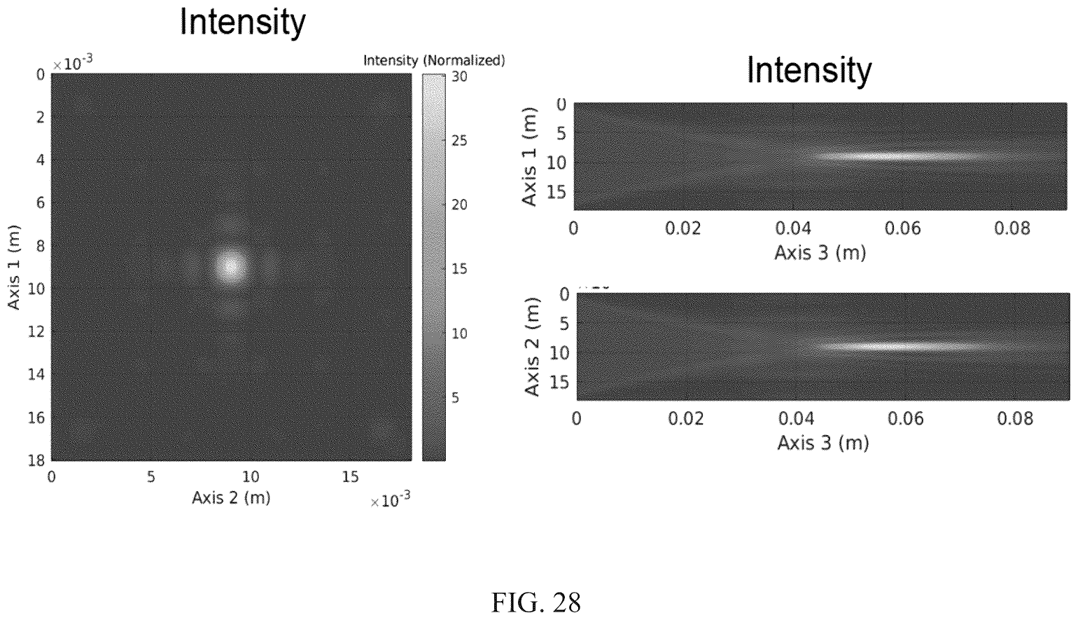

For neuromodulation, transmit timing of individual elements is used to focus the ultrasound energy to a specific location. This focusing leads to constructive interference increasing acoustic pressure (and hence intensity) that can be characterized by the focal gain of the system. To characterize this focal gain, numerical simulations of our 112×112 150-micron pitch transducer array were performed. A homogeneous medium with an attenuation coefficient of 0.5 dB/cm/MHz and a transmit frequency of 4 MHz was assumed. A focal gain, in terms of acoustic pressure, of 9.72, 6.15, and 4.38 for depths of 4, 6, and 8 cm, was computed. In the case of the 6 cm focal point, the focal gain indicates that the pressure at the focal point is 6.15 times the pressure at the transducer surface. FIG. 28 shows cross-sections of the simulation output illustrating the intensity profile of a single transducer for a focal depth of 6 cm.

The intensity of an ultrasound wave is proportional to the square of the pressure, thus assuming a focal gain for pressure of G, the focal intensity (If_spta) can be calculated as If_spta=G²×Is_spta, given that I=P²/(2×ρ×c), where I is intensity, P is pressure, ρ is density, and c is speed of sound. If the following terms are defined as the focal gain for pressure and the focal gain for intensity,

$$G\_P = P\_focused / P\_surface$$

$$G\_I = I\_focused / I\_surface$$

then we can substitute the intensity formula into the definition of G_I to find:

$$G\_I = ( {P\_focused}^{2} / 2 ρ v ) / ( {P\_surface}^{2} / 2 ρ v )$$

$$G\_I = {P\_focused}^{2} / {P\_surface}^{2}$$

$$G\_I = {G\_P}^{2}$$

Thus:

$$If\_spta ( 4 cm ) = {G}^{2} \times Is\_spta = 9.7 {2}^{2} \times 0.0806 W / {cm}^{2} = 7.6 W / {cm}^{2}$$

$$If\_spta ( 6 cm ) = {G}^{2} \times Is\_spta = 6.1 {5}^{2} \times 0.0806 W / {cm}^{2} = 3.1 W / {cm}^{2}$$

$$If\_spta ( 8 cm ) = {G}^{2} \times Is\_spta = 4.3 {8}^{2} \times 0.0806 W / {cm}^{2} = 1.5 W / {cm}^{2}$$

Taking into account that the system worked in the present example may involve three such arrays, the total focal intensity (If_spta) for these distances can be approximated as:

$$If\_spta ( 4 cm ) = 68.6$$

$$If\_spta ( 6 cm ) = 27.4$$

$$If\_spta ( 8 cm ) = 13.9$$

The above approximations are possible, given that when multiple pressure waves are incident on the same point and their phases are aligned, the resulting total pressure is the linear sum of the individual pressure amplitudes (through the same constructive interference that allows for focusing with phased arrays). Given this relationship, the net intensity of the three transducers can be computed as:

$$I\_total = {( P\_total )}^{2} / ( 2 \times ρ \times c )$$

where P_total is the total pressure due to the three transducers and is equal to P_trans1+P_trans2+P_trans3 based on the linearity assumption. Further, assuming the pressure due to each transducer is equal, the relationship can be written as 3×P_single_transducer. Note that the assumption of equal pressure is unlikely in practice, because transducers can most likely be at different distances, and can travel through different media, but this unlikely assumption represents a reasonable first order approximation. From these considerations:

$$I\_total = {( 3 \times P\_single \_transducer )}^{2} / ( 2 \times ρ \times c ) = 9 \times {( P\_single \_transducer )}^{2} / ( 2 \times ρ \times c )$$

The pressure at the single transducer is related to the intensity as

$$P\_single {\_trasnducer}^{2} = I\_single \_tansducer \times 2 \times rho \times c$$

Substitution and cancellation leaves:

$$I\_total = 9 \times I\_single \_transducer$$

Or, more generally: I_total=num_transducers²×I_single_transducer.

Comparing these values against our table of effective neuromodulation parameters demonstrates that at least twice the maximum intensity can be achieved, even at depths of 8 cm. Thus, the thermal budget of the implant allows for effective neuromodulation spatial peak temporal average intensities. Additional factors, such as heterogeneity of the media and relative spatial locations of the transducers with respect to the focal location can impact these values but given that we have a significant margin of error, these factors should not prove prohibitive.

Another aspect of feasibility is whether sufficient sustained power to the transducers to maintain neuromodulation intensity over the pulse durations (e.g., on the order of 0.5-30 ms, duty cycled) and total therapeutic window can be achieved. For this indicative feasibility calculation, the highest power neuromodulation parameter set, a challenging scenario with a pulse duration of 30 milliseconds (ms), a duty-cycle of 30%, a total therapeutic window of 40 s, and spatial peak pulse average intensity (I_sppa) of 25 W/cm² is used at our focal location. Inverting the calculations described above (e.g., moving from a desired focal intensity to an electric power level) yields a target peak power of 584 mW for each cranial implant. The per transducer electric power is:

$$If\_sppa = If\_sppa \_total / {num\_trasducers}^{2}$$

The per transducer surface If_sppa as:

$$I\_surface = If\_sppa / {Gp}^{2}$$

The acoustic power as:

$$Pa = I\_surface \times A$$

The electric power as:

$$Pe = P a / Eff ;$$

When a target If_sppa of 25 W/cm² is used, a required 584 mW of electric power is derived.

A single Li battery with capacity of 3000-4000 mAh powering up to three cranial implants could supply the neuromodulatory current without meaningful voltage droop or capacity degradation. Finally, additional parameters may affect ultrasound neuromodulation. For example, tissue heterogeneity may distort individual beams, with consequences on focal gain and beam alignment across transducers. This latter effect can also depend on multi-transducer alignment. Importantly, imaging and neuromodulation can be performed using the same arrays, and thus, whatever distortion is introduced by the heterogeneous acoustical path can be mapped to imaging space. Thus, when targeting a focal location defined in imaging space with the same array used for imaging, distortion is effectively automatically compensated for. In summary, the feasibility analysis articulated herein indicates that the disclosed systems can match even the most stringent acoustic parameters that have been demonstrated to provide effective neuromodulation.

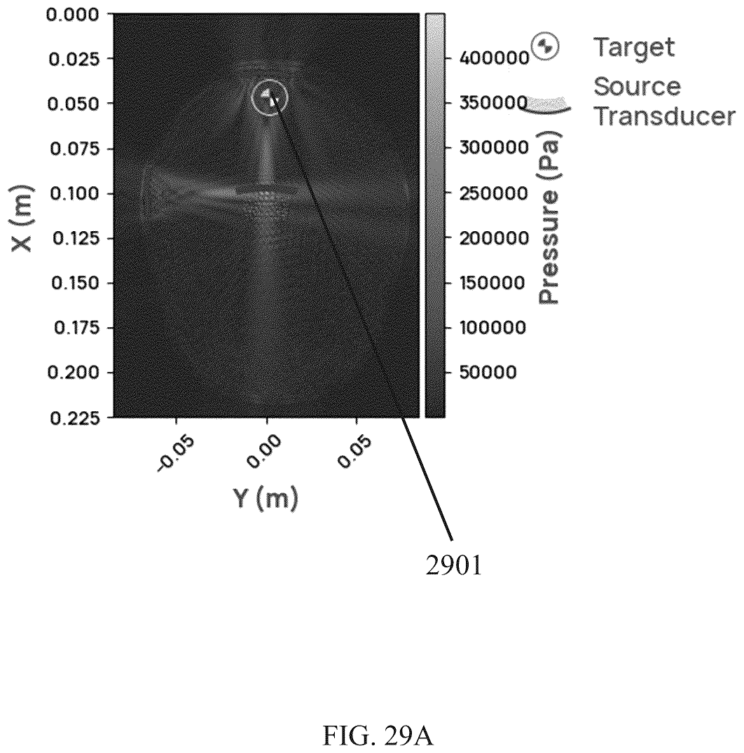

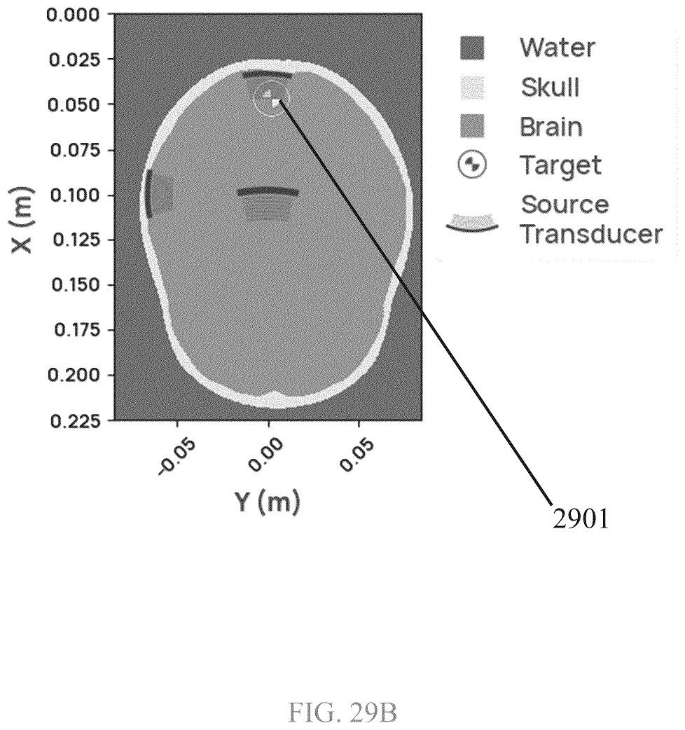

FIGS. 29A and 29B provide color-mapped plots of pressure, when one or more ultrasound transducers apply ultrasound waves to a skull of a human subject. FIG. 29A provides a simulated quantitative 2D plot that depicts the pressure applied from ultrasound waves emanating from an ultrasound transducer to a human skull. FIG. 29B provides a mapping of the materials used to generate the simulation results shown in FIG. 29A, such that the plot shown in FIG. 29B can be overlaid onto the plot shown in FIG. 29A. For both FIGS. 29A and 29B, the skull is depicted as a 2D projection from a top-down view, and a single 2D slice along the z-axis is depicted. For both FIGS. 29A and 29B, the y-axis of the plot depicts the x dimension in meters, and the x-axis of the plot depicts the y dimension in meters, such that the 0.00 m position of the y dimension in meters is centered on the midpoint of the y dimension in meters. For FIG. 29A, a color bar is shown on the right side of the plot, which quantifies the pressure in Pa across space, as a function of color. The color bar ranges from less than 50000 Pa to more than 400000 Pa. FIG. 29A depicts the pressure field during ultrasound neuromodulation of the subject's brain, when three ultrasound transducers are applying ultrasound waves to the brain of the subject. FIG. 29B depicts three different media through which the ultrasound waves transmit, to generate the pressure field mapping in FIG. 29A. The three different media are water, the subject's skull, and the subject's brain. A circle with two quadrants 2901 in the middle of the plot represents the target on which pressure is applied.

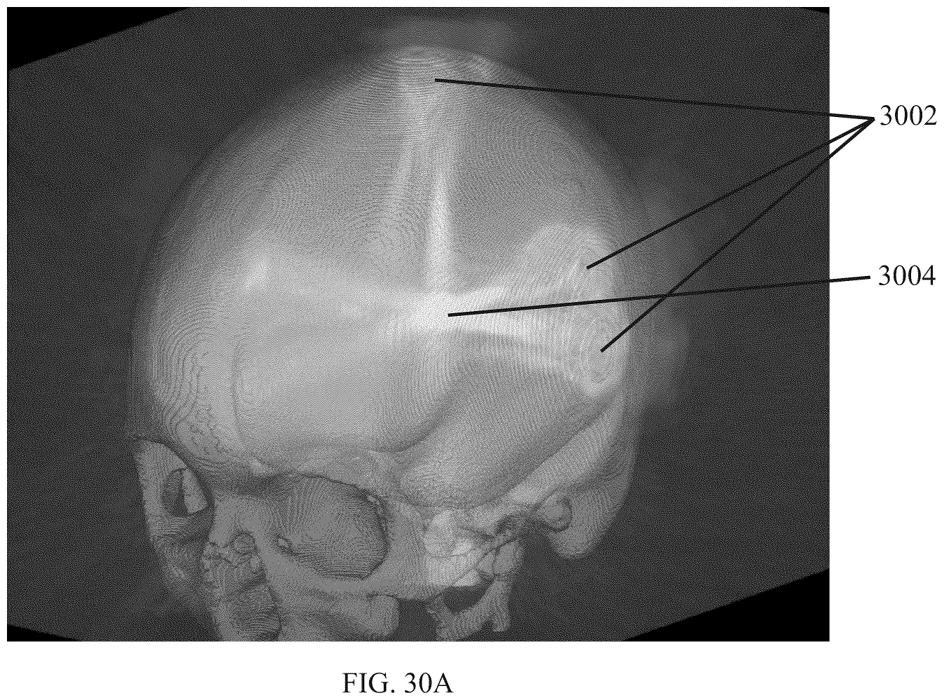

FIG. 30A provides a simulated 3D plot of pressures when applying ultrasound from non-implantable ultrasound transducer devices to a human subject. FIG. 30A depicts the strength of pressures relative to other regions of the plot. FIG. 30A shows three ultrasound emanation points 3002, each of which correspond to the position of one of three ultrasound transducers. The emanated ultrasound waves intersect at hotspot 3004, where constructive interference from the ultrasound waves emanating from the three emanation points occur. Such an intersection of the ultrasound waves is also depicted in FIG. 15D, which refers to the intersection of the ultrasound waves as beam intersection. The location of the hotspot 3004 is deep in the interior of the skull of the subject, which demonstrates the capacity of each ultrasound transducer to deliver ultrasound waves deep into the brain of the subject. The simulated 3D pressure plot shown in FIG. 30A was generated in Neurotech Development Kit (Los Angeles, California), an open-source simulation software.

FIG. 30B provides a simulated 3D plot of pressures when applying ultrasound from implantable transducer devices to a subject. FIG. 30B depicts the strength of pressures relative to other regions of the plot. FIG. 30B depicts the pressures for regions both interior and exterior to the skull of the subject (the brain is interior to the skull of the subject). FIG. 30B shows three implantable ultrasound transducers 3006, implanted into the skull of the subject. The spatial distribution of the pressure values from the ultrasound waves emanating from the three implantable ultrasound transducers were plotted. The three implantable transducers can, for example, comprise the miniature implantable ultrasound transducers depicted in FIG. 5. The strongest pressure values for the simulation were observed at the hotspot 3008, where each of three hotspots corresponding to each of three implantable transducers intersect, thereby achieving constructive interference across the ultrasound waves and generating hotspot 3008. Such an intersection of the ultrasound waves is also depicted in FIG. 15D, which refers to the intersection of the ultrasound waves as beam intersection. The location of the hotspot 3008 is deep in the interior of the skull of the subject, which demonstrates the capacity of each ultrasound transducer to deliver ultrasound waves deep into the brain of the subject. The simulated 3D pressure plot shown in FIG. 30B was generated in Neurotech Development Kit (Los Angeles, California), an open-source simulation software.

### Example 5—Imaging Compatibility Analysis for a Sonolucent Window

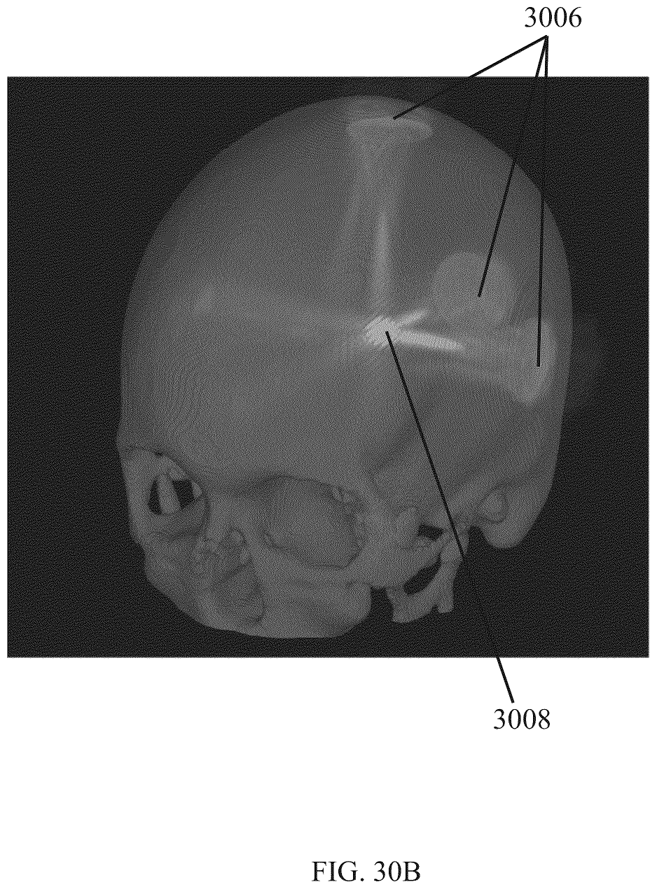

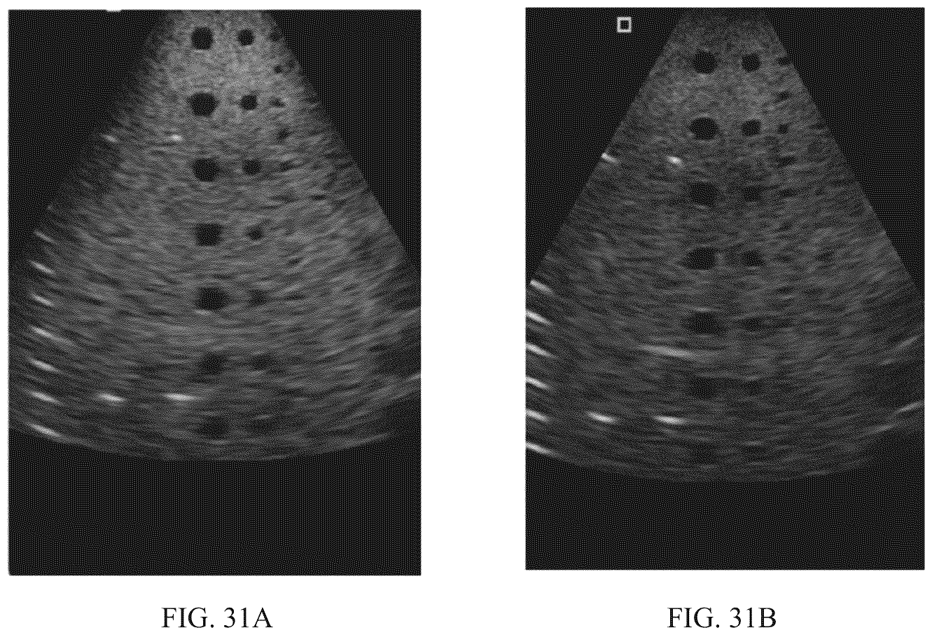

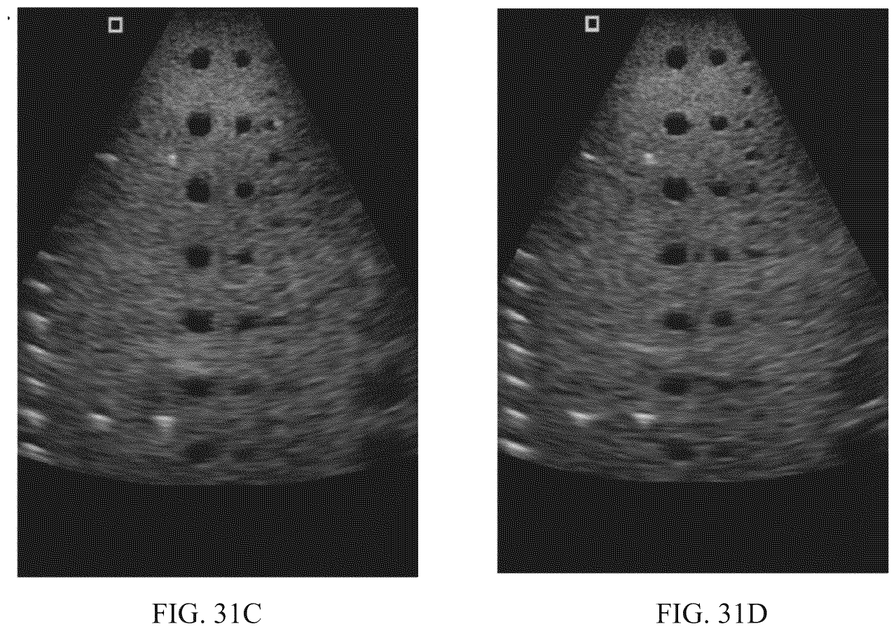

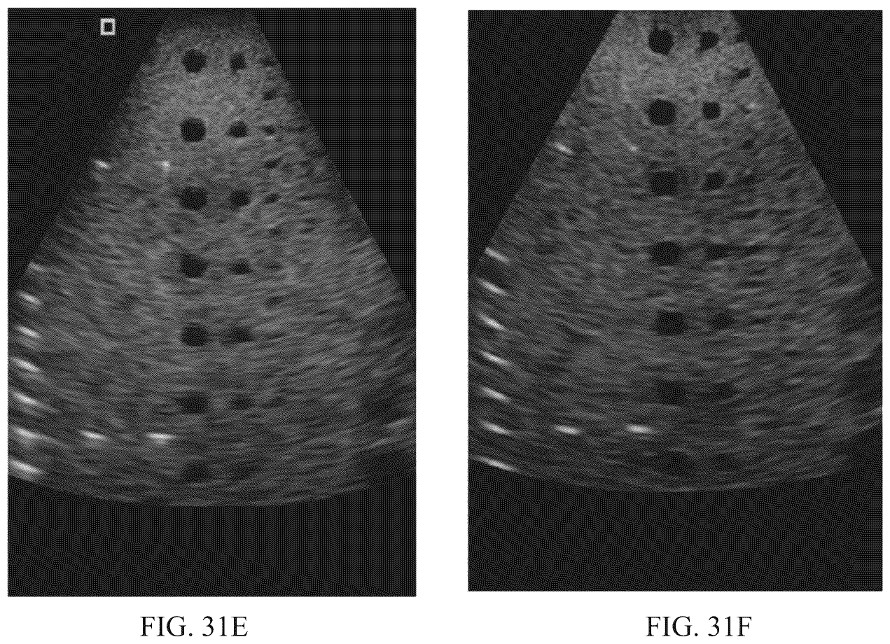

FIGS. 31A-31G provides data describing the ultrasound B-mode imaging quality and transparency of an ultrasound transducer comprising one of several sonolucent materials. Each figure of FIGS. 31A-31G demonstrates the imaging quality transparency of the ultrasound transducer through a lens made of a specific sonolucent material. The data shown in FIGS. 31A-31G are images of an imaging phantom configured for B-mode ultrasound imaging. The imaging phantom can serve as an object configured to operate as a substitute for human tissue, when imaged by an imaging modality, such as ultrasound imaging, e.g., focused ultrasound imaging. Thus, each figure in FIGS. 31A-31G showcases the efficacy of each of the sonolucent materials, with respect to maintaining clear ultrasound signal transmission. The ultrasound transducer used for generating the data depicted in FIGS. 31A-31G comprised a hermetically sealed housing designed to fit within the cranial area, with dimensions ranging from 3 mm to 20 mm in thickness, and 14 mm to 50 mm in width or length. The enclosure was designed such that it could accommodate a cable for unidirectional power and bidirectional data transmission.

FIG. 31A shows imaging data of the imaging phantom when the ultrasound transducer emanates ultrasound waves through ultra-high-molecular-weight polyethylene (UHMW-PE). FIG. 31B shows imaging data of the imaging phantom when the ultrasound transducer emanates ultrasound waves through PTFE. FIG. 31C shows imaging data of the imaging phantom when the ultrasound transducer emanates ultrasound waves through polymethyl methacrylate (PMMA). FIG. 31D shows imaging data of the imaging phantom when the ultrasound transducer emanates ultrasound waves through polyethylene terephthalate (PET). FIG. 31E shows imaging data of the imaging phantom when the ultrasound transducer emanates ultrasound waves through polyether ether ketone (PEEK). FIG. 31F shows imaging data of the imaging phantom when the ultrasound transducer emanates ultrasound waves through polyether block amide (PEBAX). FIG. 31G shows imaging data of the imaging phantom when the ultrasound transducer emanates ultrasound waves through high density polyethylene (HDPE).

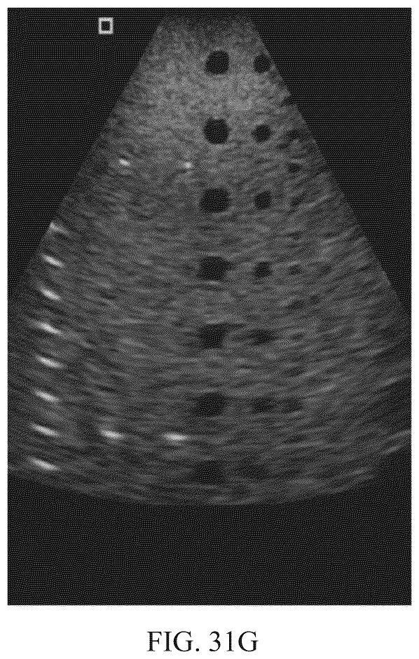

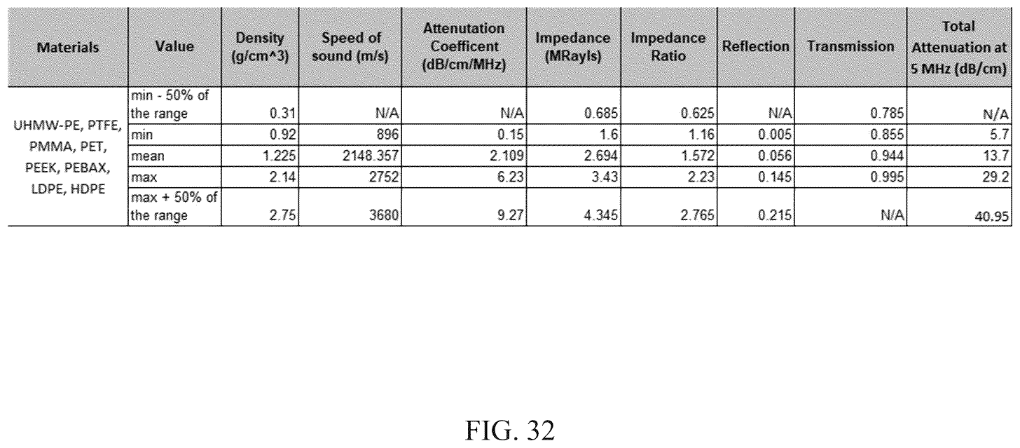

FIG. 32 provides tabular data describing the physical properties of ultrasound waves passing through one of several sonolucent materials. Both theoretical data and experimental data are shown for the following sonolucent materials: UHMW-PE, PTFE, PMMA, PET, PEEK, PEBAX, LDPE, HDPE. For each of the sonolucent materials, the table provides values for the density, speed of sound (e.g., the speed of sound when sound waves, such as ultrasound waves, transmit through the sonolucent material), attenuation coefficient, impedance (e.g., impedance coefficient), impedance ratio, reflection (e.g., reflection coefficient), transmission (e.g., transmission coefficient), and the total attenuation of ultrasound waves comprising a frequency of 5 MHz. The values provided for each of these properties comprise a) the minimum minus 50% of the range, where the range is the difference between the minimum and maximum values for a given property; b) the minimum; c) the mean; d) the maximum; and e) the maximum plus 50% of the range, where the range is the difference between the minimum and maximum values for the given property. The “N/A” value in the tabular data of FIG. 32 refers to the value being not applicable. The impedance ratio shown in FIG. 32 can represent the ratio of the acoustic impedance of a material to the acoustic impedance of a reference medium, which in the case of FIG. 32, is water. The total attenuation of ultrasound waves comprising a frequency of 5 MHz refers to the extent of attenuation of the ultrasound waves as they travel across a distance of the sonolucent material. The thickness of the sonolucent material is not accounted for, when determining the total attenuation of the ultrasound waves.

### Example 6—Feasibility Analysis for Modulating Nervous Systems

The present example describes experiments used for validating the use of ultrasound transducers configured for imaging and/or modulating the nervous system. The validating can comprise, for example, quantifying the physical properties of ultrasound waves emanating from one or more ultrasound transducers. Quantifying the physical properties of the ultrasound waves can be for ultrasound waves that emanate from one or more ultrasound transducers when the ultrasound transducers are configured for imaging or modulating the nervous system of a subject. The ultrasound waves from the ultrasound transducer configured to neuromodulate a subject can comprise the delivery of focused ultrasound waves.

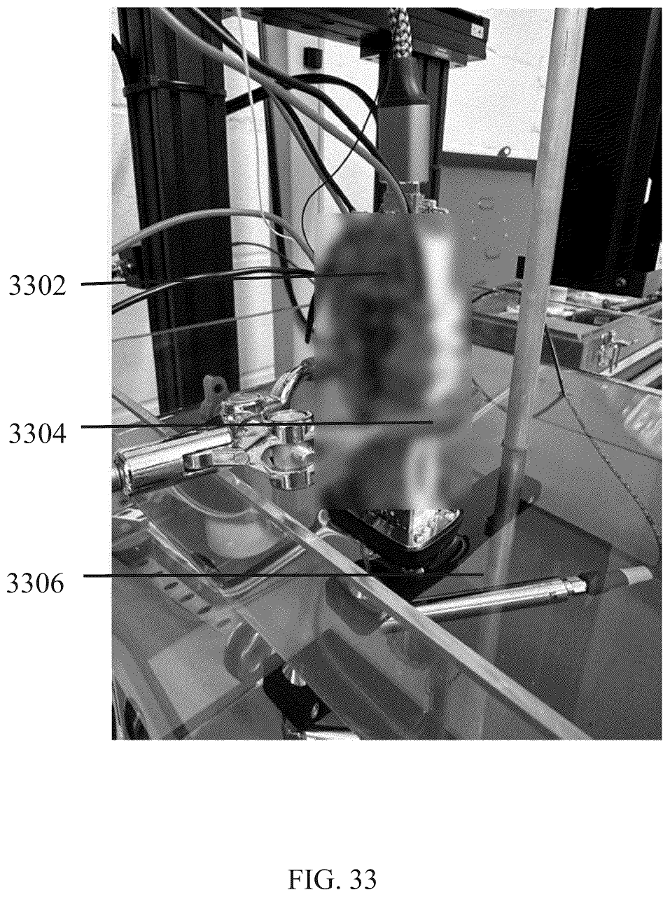

FIG. 33 depicts an image of an experimental chamber, where an ultrasound transducer is configured to apply ultrasound waves into a water tank 3306. The ultrasound transducer depicted in FIG. 33 was modified for modulating the nervous system, rather than imaging the nervous system, in part by adding additional capacitors not present in the circuitry for an ultrasound transducer configured for imaging the nervous system. The ultrasound transducer is not configured for implanting into the skull of a subject, but rather, is configured for use outside of the skull of the subject. The ultrasound transducer can be manually held and/or positioned by a clinician, e.g., a surgeon or a surgical assistant, or by a mechanical apparatus, such as the clamp stand depicted in FIG. 33. FIG. 33 depicts the ultrasound transducer 3302, secured by a clamp 3304. The ultrasound transducer 3302 is oriented such that the MEMS ultrasound array is pointed towards a water tank 3306. The water tank can be used to approximate the impedance encountered by ultrasound waves when the ultrasound waves are applied to a human brain. The ultrasound transducer can be in contact with the surface of the water tank, as depicted in FIG. 33.

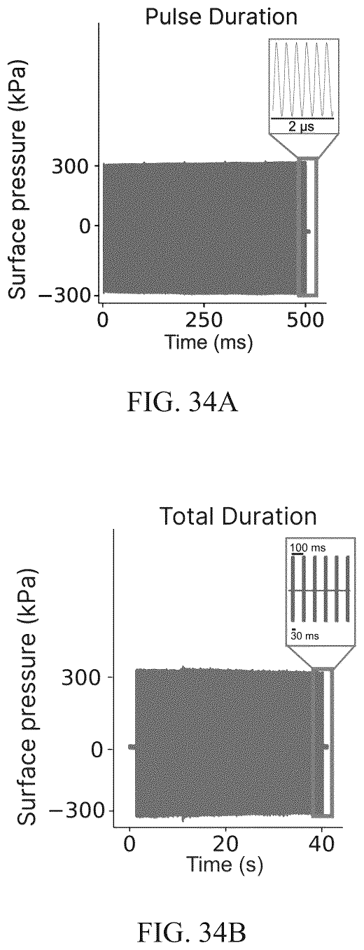

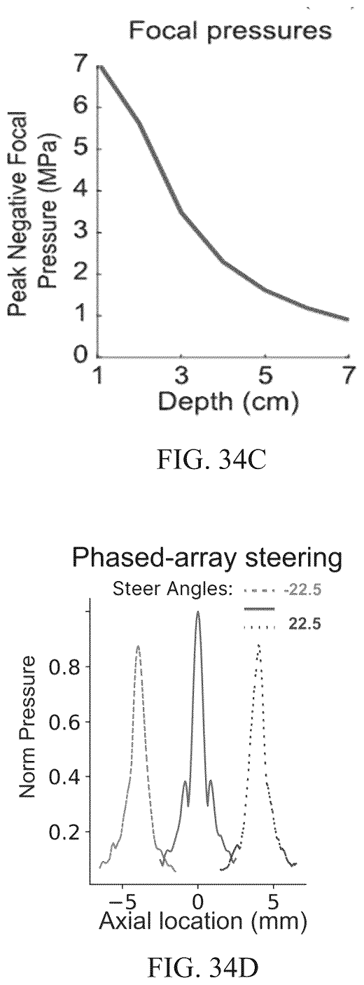

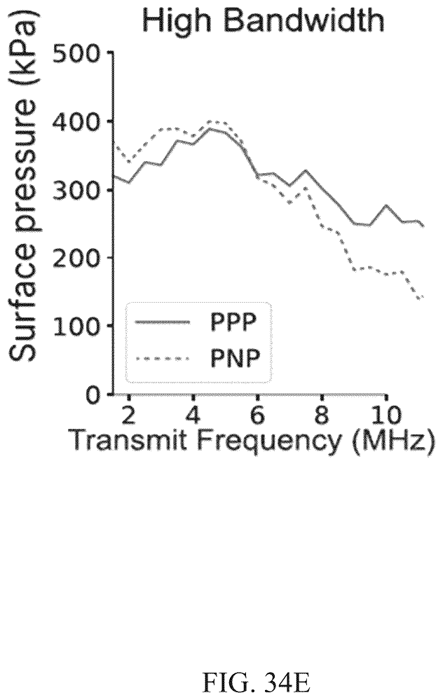

FIGS. 34A-34E demonstrates the ultrasound transducer's ability to provide ultrasound waves capable of modulating the nervous system of a subject. The data depicted in FIGS. 34A-34E was based on an experimental chamber for testing ultrasound transducer properties, such as the experimental chamber shown in FIG. 33. In addition, the ultrasound transducer was modified for modulating the nervous system, rather than imaging the nervous system, in part by adding additional capacitors not present in the circuitry for an ultrasound transducer configured for imaging the nervous system. A target set of physical ultrasound wave parameters for modulating the nervous system of a subject was selected. The target set of ultrasound wave parameters can comprise a pulse length of at least approximately 300 ms, a focused pressure of at least approximately 1 MPa, and a frequency of at least approximately 5 MHz. From the experimental chamber used to test the ultrasound waves, as shown in FIG. 33, the physical ultrasound wave parameters from the modified ultrasound transducer were measured using hydrophone surface pressure measurements with an unfocused transmit at 3 MHz.

FIG. 34A shows long pulse durations for ultrasound waves from an ultrasound transducer configured for neuromodulating a subject. FIG. 34A shows long pulse durations suitable for real-time, e.g., online, neuromodulation, as indicated, in part, by the sustained pressures over extended periods. FIG. 34B shows sequences with long total durations of ultrasound waves suitable for non-real-time, e.g., offline, neuromodulation. FIG. 34C shows the peak negative focal pressure in MPa, as a function of distance in cm. A phase array of the ultrasound transducer can focus energy to achieve high focal pressure as a function of depth, given an attenuation of 0.7 dB/MHz/cm. The ability for the ultrasound transducer to deliver high focal pressure permits effective neuromodulation for tissue at specific depths, such as those depths indicative of the distance between an implanted ultrasound transducer and an inferior region of a human brain. FIG. 34D depicts the normalized pressure at various phased-array steering angles for ultrasound waves emanating from an ultrasound transducer configured for neuromodulating a subject. The ultrasound transducer can deliver ultrasound waves, e.g., ultrasound beams, at steering angles of −22.5 degrees, 0 degrees, and 22.5 degrees. The normalized pressures are measured as a function of the axial location, in mm. FIG. 34E depicts the bandwidth at which the ultrasound waves can operate for an ultrasound transducer configured for neuromodulating a subject. The surface pressure in kPa across a range of transmit frequencies ranging from 2 to 10 MHz is depicted for both peak positive pressure (PPP) and peak negative pressure (PNP). FIG. 34E demonstrates that a broad range of ultrasound transmit frequencies can be used to achieve high surface pressures.

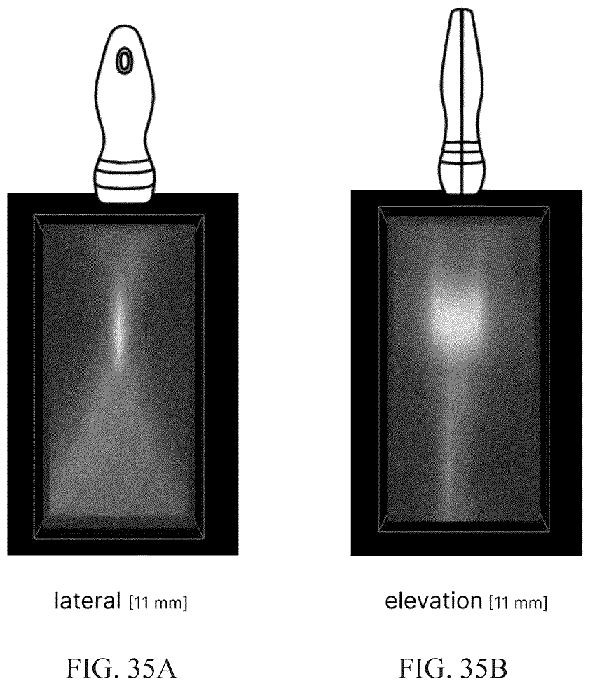

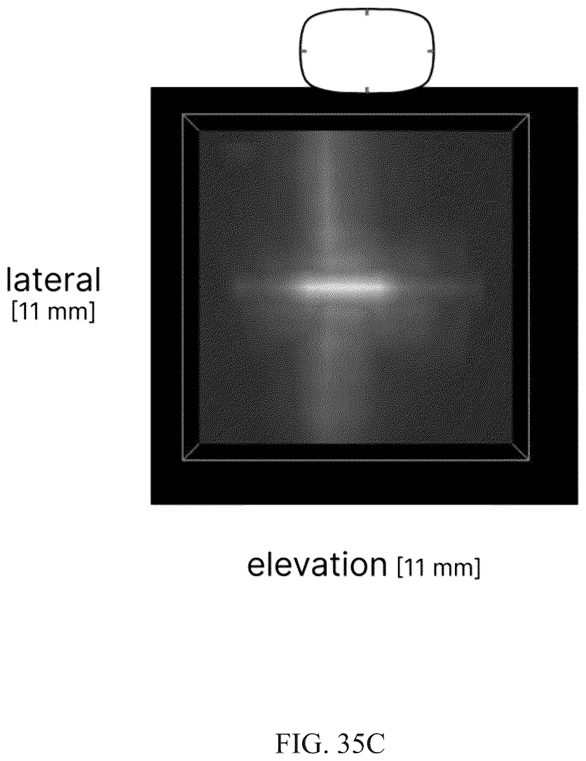

FIGS. 35A-35C provide 2D color-mapped plots of the ultrasound waves, e.g., ultrasound pressures, emanating from the modified ultrasound transducer, as a function of space, where the space maps onto a volume. The data depicted in FIGS. 35A-35C result from hydrophone data experimentally collected from a water tank. The color-mapped values represent the pressure value, e.g., in Pascals, of the delivered ultrasound waves. The data shown across FIGS. 35A-35C, when combined, provides a 3D depiction of the spatial distribution of ultrasound pressures, when an ultrasound wave is emanated from the ultrasound transducer. FIG. 35A depicts a spatial distribution of ultrasound pressures in the volume, where the lateral dimension is 11 mm and the depth dimension is 21 mm. FIG. 35B depicts a spatial distribution of ultrasound pressures in the volume, where the elevation is 11 mm and the depth is 21 mm. FIG. 35C depicts a spatial distribution of ultrasound pressures in the volume, where the lateral dimension is 11 m and the elevation dimension is also 11 mm. Taken together, FIGS. 35A-35C show that the spatial distribution of the highest pressure values of the ultrasound wave resembled a rectangular 3D-sheet structure, wherein the ‘thickness’ of the sheet can be seen across the lateral dimension, the ‘length’ of the sheet can be seen across the depth dimension, and the ‘width’ of the sheet can be seen across the elevation dimension. The hydrophone data shown in FIGS. 35A-35C provide feasibility analysis for not only ultrasound for neuromodulating a subject, but also for ultrasound imaging a subject.

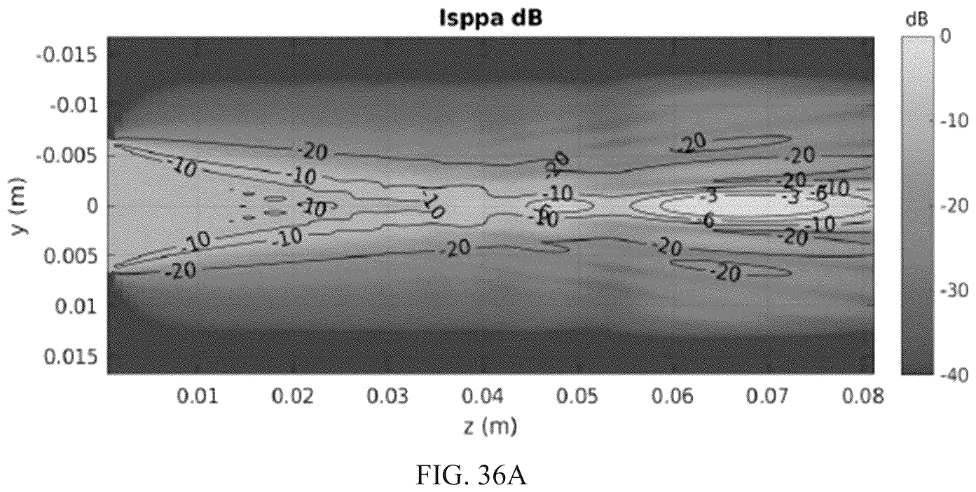

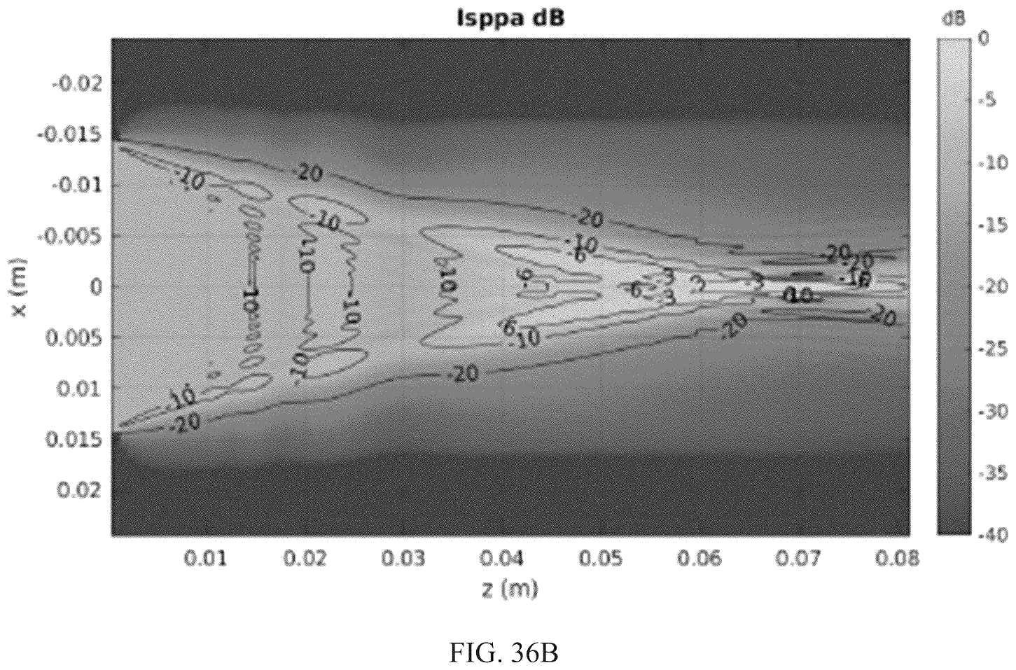

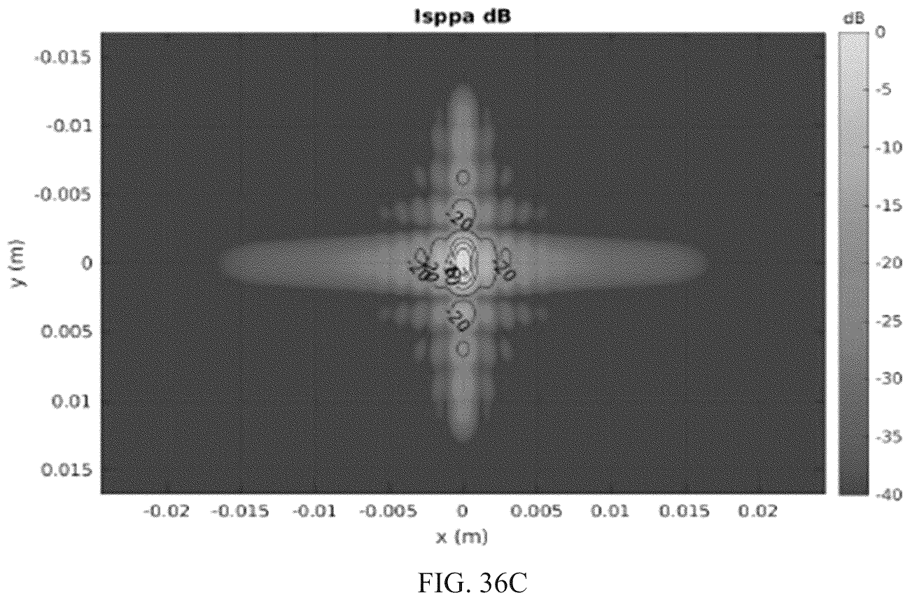

FIGS. 36A-36C provide simulation data of one or more ultrasound pulses as a function of space, in m, when delivered by an implantable ultrasound transducer device. For each of FIGS. 36A-36C, the spatial distribution of the intensity of spatial peak pulse average (ISPPA or I_sppa) is shown in dB for ultrasound waves. The simulation data shown in FIGS. 36A-36C provide feasibility analysis for not only ultrasound-based neuromodulating of a subject, but also for ultrasound-based imaging a subject. FIG. 36A depicts the spatial distribution of the ISPPA intensity across y and z length dimensions. FIG. 36B depicts the spatial distribution of the ISPPA intensity across x and z length dimensions. FIG. 36C depicts the spatial distribution of the ISPPA intensity across y and x length dimensions.

### Example 7—Correlating Stimulus Response and Ultrasound-Based Neural Activity of a Subject

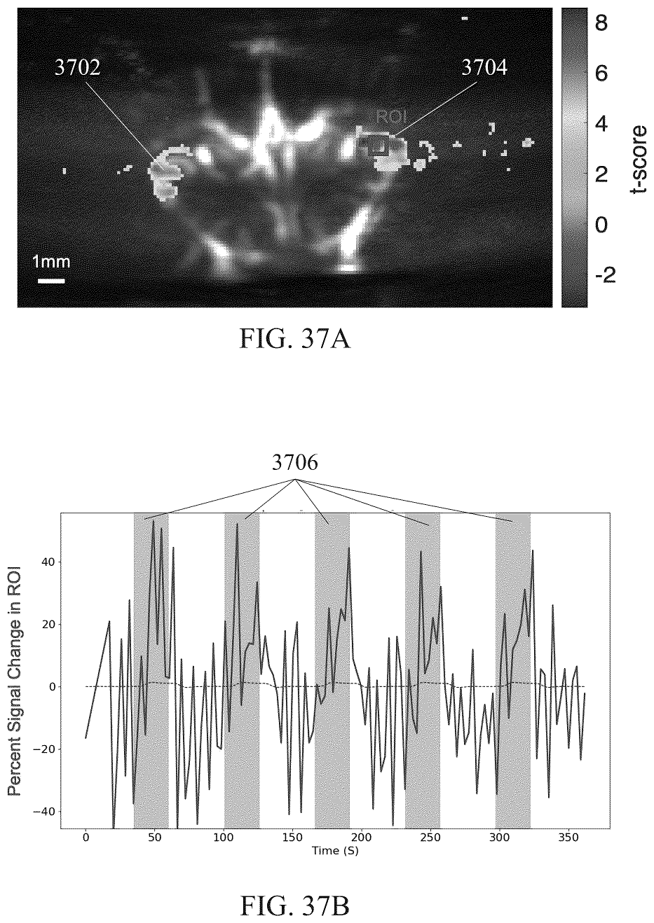

FIG. 37A depicts an activity map resulting from ultrasound imaging of a coronal section in the brain of a female Long-Evans rat, while the rat was presented visual stimuli. The results of FIG. 37A show the example advantages of the methods and systems disclosed herein over traditional neurotechnologies for imaging neural activity. The regions of the brain indicated by pixels on the activity map may show the extent to which the visual stimulation of the subject and ultrasound imaging are correlated. As illustrated, the x- and y-axes of the figure are in millimeters, and they depict the ultrasound imaging region of interest, of the brain of the subject. The color bar depicts the statistical t-score. A generalized linear model (GLM) was used to show the relationship between brain activity in the brain of a rat while the subject underwent periods of visual stimulation. The GLM was fitted to the timeseries of individual image pixels' magnitude derived from functional ultrasound imaging of the brain. The pixels that better predicted the task outcome of the subject, e.g., visual stimulus presentation regimen, received a higher t-score. t-scores were mapped in accordance with the color bar. The resulting hotspots 3702 and 3704 of high t-score values were consistent with electrophysiological and fMRI literature results indicating that the lateral geniculate nucleus was active during visual stimulation. The background image in black and white was a static vascular image overlaid against the visualized t-scores.

FIG. 37B depicts a timeseries trace for the region of interest (ROI) on the hotspot 3704 on FIG. 37A. The timeseries trace depicts the percent signal change in neural activity in the ROI on the y-axis, and the time, in seconds, on the x-axis, over the course of more than 350 seconds. The grey bars 3706 indicate the durations of time over which the visual stimulus was provided to the rat. The percent signal change in the ROI increases during the durations of visual stimulus presentation, relative to the durations of no visual stimulus presentation.

In some embodiments, a method for treating a nervous system of a subject comprised receiving, from an implantable transducer, ultrasound data of the nervous system. The ultrasound data indicate physiological state of the nervous system. The method further comprises processing the ultrasound data and transmitting the processed ultrasound data. Instructions for modulating neural activity of the nervous system are determined based on the processed ultrasound data. In some embodiments, the ultrasound data can comprise one or more ultrasound images.

### Example 8—Ultrasound-Based Imaging of a Human Nervous System

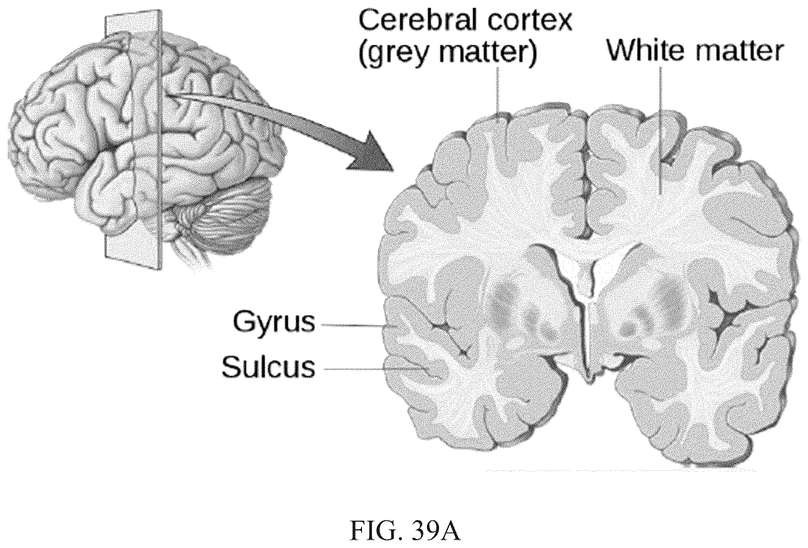

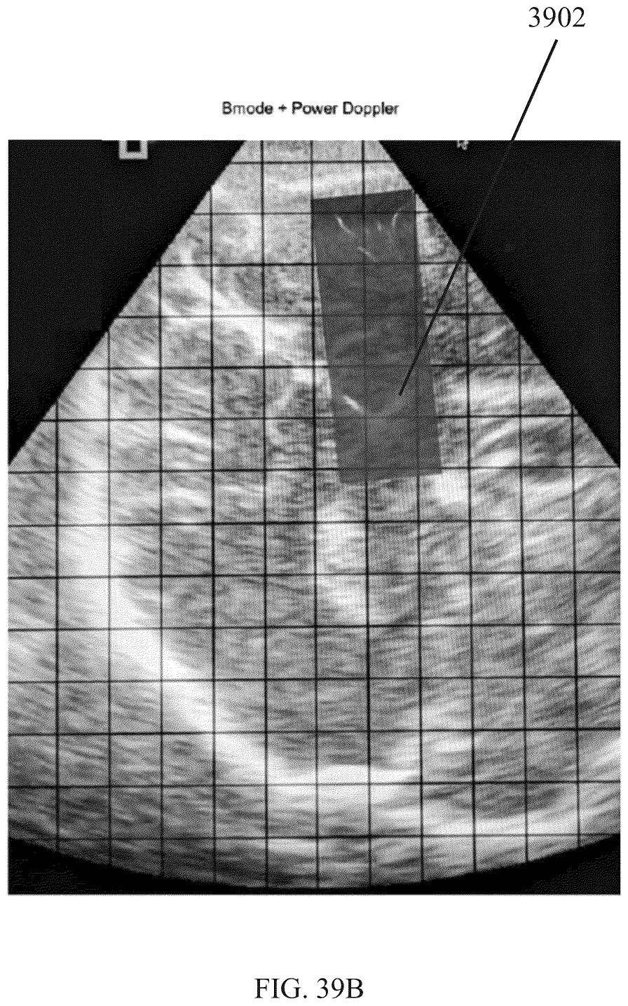

FIGS. 38-42B provide data demonstrating ultrasound-based imaging of nervous systems from humans, using one or more ultrasound transducers. FIGS. 38-42B demonstrate that ultrasound transducers can obtain ultrafast ultrasound sequences at frequencies of 1 kHz or higher, anatomical, e.g., B-mode, imaging in humans, power Doppler imaging in humans, and functional imaging in humans. FIG. 38 provides a schematic of a clinical set-up in which a non-implantable ultrasound transducer 3802, e.g., an ultrasound transducer, can be used by a surgeon and/or a mechanical implement 3804 to image neural activity from a human subject 3806. The schematic shown in FIG. 38 describes the experimental set-up used to obtain the data depicted in FIGS. 39B-42B. The intended anatomical target of the ultrasound waves emanating from the ultrasound transducer 3802 is depicted by a crosshair 3808 in the inset image of FIG. 38. That is, a superficial cortical region was the intended anatomical target of the ultrasound waves from the ultrasound transducer 3802. The ultrasound transducer 3802 can be configured to image the neural activity of the brain of the human subject 3806 and need not be configured to modulate the neural activity of the brain of the human subject 3806.

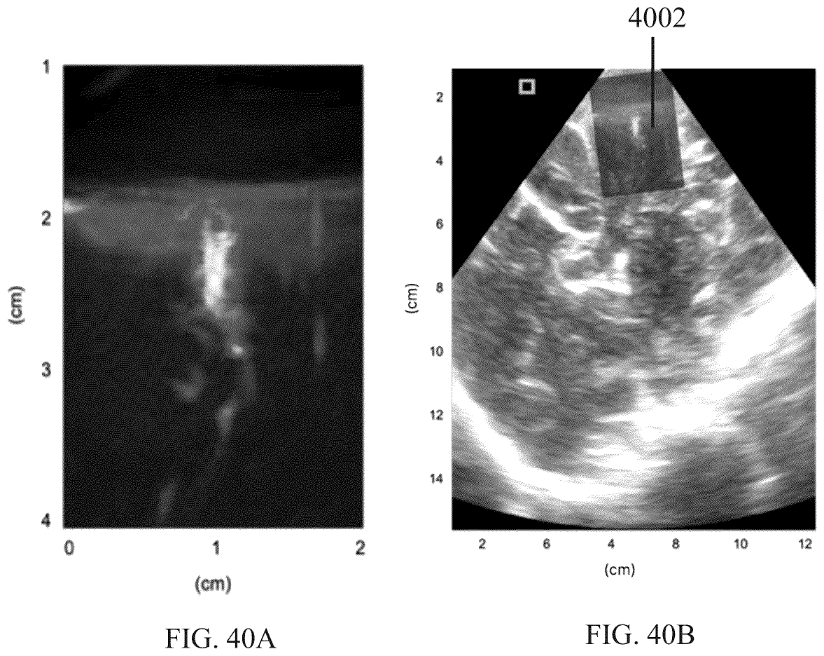

FIGS. 39A-39B provide images describing the intended anatomical target of the ultrasound waves from the ultrasound transducer 3802, based on the set-up described in FIG. 38. FIG. 39A provides a schematic of a coronal section of the human brain. Accordingly, a coronal section from the ultrasound imaging performed as shown in FIG. 38 is shown in FIG. 40B. The functional ultrasound imaging was targeted at approximately on the region of interest 3902. The rest of the image, e.g., the parts of FIG. 39B not comprised in 3902 is an anatomical image of the subject's human brain and skull, and was acquired via B-mode ultrasound imaging. The region of interest 3902 can resemble the volume geometry of functional ultrasound imaging depicted in FIGS. 36A-36C. The color-mapped intensity values shown in the region of interest 3902 correspond to intensity values that can represent the neural activity of the human subject at one or more approximate moments in time. In the case that multiple approximate moments in time are depicted on a static image, the data collected across the moments in time can be collapsed such that a compiled value (e.g., summed, averaged, or maxed value from the time range corresponding to the moments in time) is generated for the time range.

For FIG. 39B, as well as FIGS. 40A-43D, intensity values from ultrasound imaging, e.g., ultrasound imaging of humans, are depicted. The intensity values can represent neural activity data, which can be based on radiofrequency data that is read by the ultrasound transducer, and the radiofrequency data can be converted into in-phase and quadrature (IQ) data, which can be converted into beamformed IQ data, from which the magnitude of the power Doppler data can be determined, which can be equivalent to the intensity values representing changes in cerebral blood volume, which are correlated to neural activity data for images derived from ultrasound transducers. These data transformations also relate to the formation of compound images. That is, in planewave imaging, such as ultrasound imaging, multiple images are formed by transmitting acoustic energy into the medium at different angles. To form a compound image, backscattered echoes from the transmitted acoustic energy are received, and the backscattered echoes are then separately beamformed and summed together. The magnitude of the image can then be computed and log-compressed to form the B-mode image. An ensemble of compound images can then be filtered to extract the power Doppler image, such that noise such as motion data deriving from non-blood motion, e.g., tissue motion, can be parsed from the blood motion, which can represent cerebral blood volume changes, and by extension, neural activity. The power Doppler images shown in at least FIGS. 39B-43D, were computed based on the data transformations described herein.

FIGS. 40A and 40B provide imaging data of neural activity from a sedated human subject's brain, obtained from ultrasound imaging with an ultrasound transducer. FIG. 40A provides power Doppler imaging data, which indicates the neural activity of a region of the brain of the subject, as a function of cerebral blood flow. The brighter pixels in FIG. 40A correspond to more active blood flow within the anatomical region. The image was acquired using an ultrasound transducer. FIG. 40B provides an anatomical B-mode ultrasound image, in conjunction with a copy of the power Doppler functional ultrasound image shown in FIG. 40A, as indicated in the region of interest 4002. Both FIGS. 40A-40B were plotted in terms of lengths in cm, as indicated by the x and y axes for both FIGS. 40A-40B.

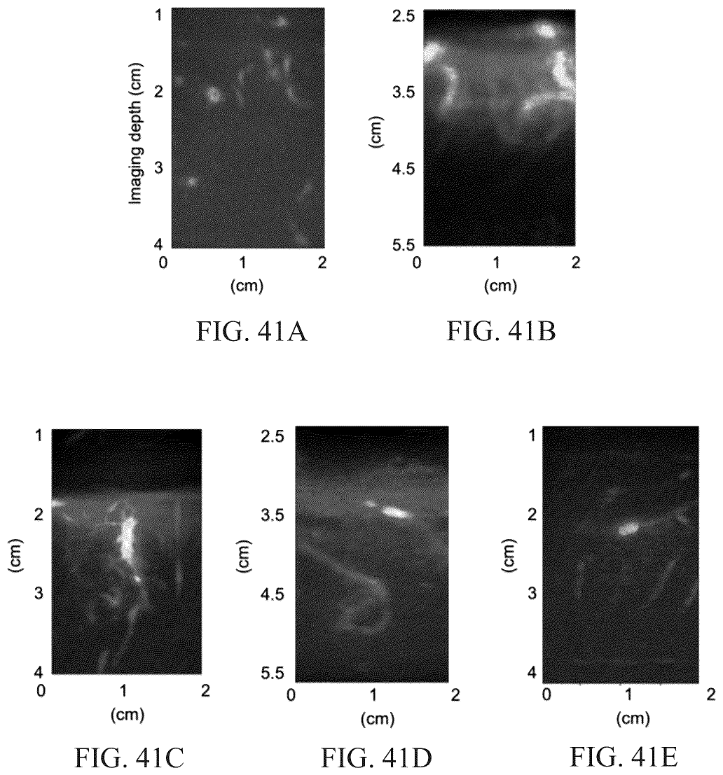

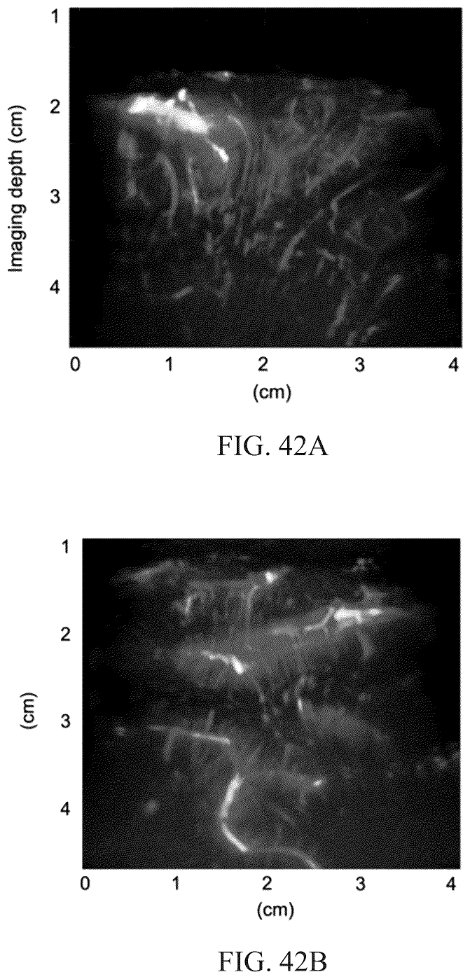

FIGS. 41A-42B provide example imaging data of neural activity from the brains of human subjects, obtained using the ultrasound transducers described in accordance with the embodiments herein. FIGS. 41A-41E provide power Doppler functional ultrasound images, which were determined according to the data transformations described herein. Each plot of power Doppler intensity values shown across FIGS. 41A-41E were derived from a distinct human subject. FIGS. 42A-42B were derived from the same human subject. The power Doppler intensity values are shown in length units of cm for both the x-axis and the y-axis, for FIGS. 41A-42B. FIGS. 41A-41E provide smaller fields of view of the power Doppler images, compared to the power Doppler intensity values shown in FIGS. 42A-42B.

### Example 9—Artificial Neural Network Reconstruction of Functional Ultrasound Images

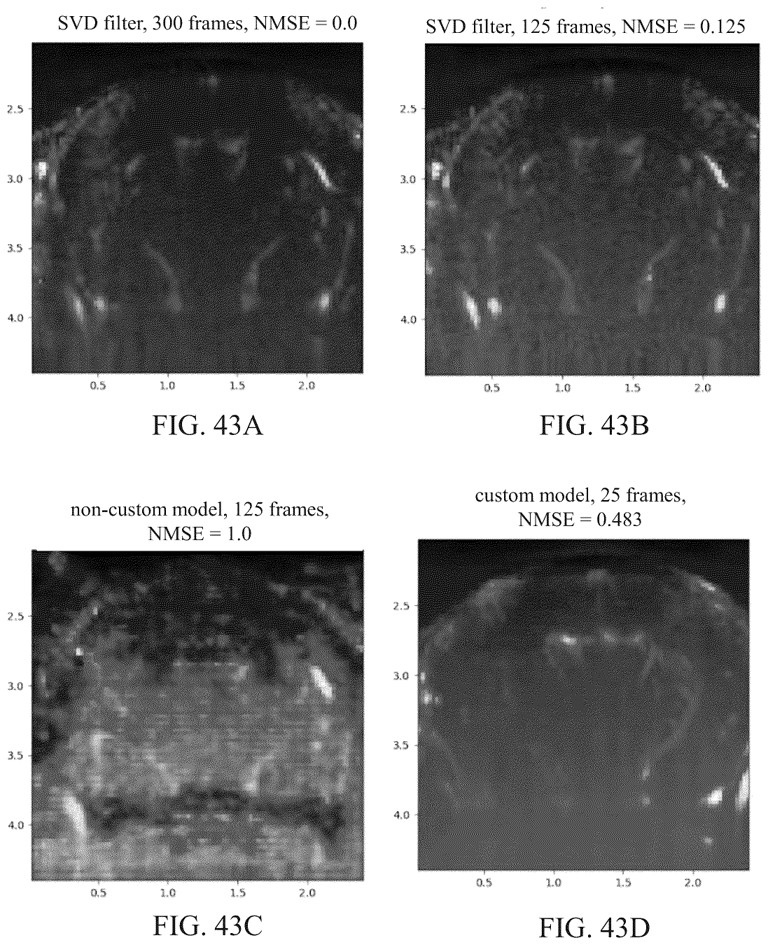

FIGS. 43A-43D provide Doppler imaging data of neural activity from a mouse brain, including reconstructed imaging data of the mouse brain, based on a trained artificial neural network (ANN) model. FIGS. 43A-43D demonstrate the use of a custom trained ANN model for reconstructing power Doppler images based on a subset of frames from a sequence of ultrasound images, e.g., a sequence of B-mode images. The custom trained ANN model can be scaled such that fewer frames from a sequence of functional ultrasound images—and accordingly, less computational resources—can be used for reconstructing the power Doppler images. FIG. 43A depicts a reference dataset that was reconstructed based on all frames, using traditional techniques—that is, techniques not comprising the use of a trained ANN model and requiring the use of all input frames relating to the power Doppler image reconstruction, which for FIG. 43A, comprised a total of 300 frames. The traditional techniques can include singular value decomposition filters. Accordingly, FIG. 43A served as a benchmark against which the results from the trained ANN model were compared. FIG. 43B shows a power Doppler image reconstructed based on the traditional techniques-namely SVD filters—using the first 125 frames of the total 300 frames. When comparing the pixel values of FIG. 43A to FIG. 43B, the normalized mean square error (NMSE) was 0.125. FIG. 43C shows a power Doppler image reconstructed based on the first 125 frames used for generating FIG. 43B, and a trained ANN described in Di Ianni and Airan, (2022), “Deep-fUS: A deep learning platform for functional ultrasound imaging of the brain using sparse data,” IEEE Transactions on Medical Imaging 41(7):1813-1825. The trained ANN described in Di Anni and Airan was trained on different training data compared to the ANN model described herein. Furthermore, the trained ANN described in Di Anni and Airan used a different cost function compared to the ANN model described herein. The first 125 frames used for generating FIG. 43B were not used to train the trained ANN as described in Di Ianni and Airan (2022). When comparing the pixel values of FIG. 43A to FIG. 43C, the NMSE was 1.0. The high NMSE indicates the poor performance of the ANN model from the Di Ianni and Airan (2022) publication. FIG. 43D shows a power Doppler image reconstructed based on 25 random frames from the 300 frames used for FIG. 43A, and a trained custom ANN. None of the first 125 frames used for generating FIGS. 43B and 44C were used to train the trained custom ANN. The trained custom ANN was trained based on imaging data from three mice subjects. When comparing the pixel values of FIG. 43A to FIG. 43D, the NMSE was 0.483. The intermediate NMSE indicates an intermediate level of performance of the ANN model. The custom ANN model provided the intermediate level of performance, as indicated by the NMSE of 0.483, using 5×fewer frames than the conventional SVD filter used for FIG. 43B (25 frames vs. 125 frames), and 5× fewer frames than the non-custom model used in FIG. 43C (25 frames vs. 125 frames). The trained custom ANN provides improved performance over the trained ANN model from the Di Anni and Airan (2022) publication.

It should be understood from the foregoing that, while particular implementations of the disclosed methods and systems have been illustrated and described, various modifications can be made thereto and are contemplated herein. It is also not intended that the disclosure be limited by the specific examples provided within the specification. While the disclosure has been described with reference to the aforementioned specification, the descriptions and illustrations of the preferable embodiments herein are not meant to be construed in a limiting sense. Furthermore, it shall be understood that all aspects of the disclosure are not limited to the specific depictions, configurations or relative proportions set forth herein which depend upon a variety of conditions and variables. Various modifications in form and detail of the embodiments of the disclosure may be apparent to a person skilled in the art. It is therefore contemplated that the disclosure shall also cover any such modifications, variations and equivalents.

What is claimed is:

1. An implantable transducer comprising:
a housing;
a sonolucent window disposed, at least in part, at a first end of the housing; and
an ultrasound array disposed within the housing proximate the first end, the ultrasound array configured to emit ultrasound waves to an outside environment via the sonolucent window.

2. The implantable transducer of claim 1, further comprising one or more one circuit boards disposed within the housing, the one or more circuit boards comprising one or more electronic components disposed thereon, the one or more electronic components and configured to send one or more signals to the ultrasound array.

3. The implantable transducer of claim 2, wherein the one or more electronic components disposed on the circuit board are configured to process data received from the ultrasound array, the data indicative of brain function in a subject.

4. The implantable transducer of claim 2, wherein the data comprises image data indicative of anatomical features of the subject.

5. The implantable transducer of claim 1, wherein the implantable transducer is configured to be disposed in a hole in a skull of the subject.

6. The implantable transducer of claim 1, wherein the implantable transducer is positioned in contact with a soft tissue of the subject.

7. The implantable transducer of claim 1, wherein the implantable transducer is positioned in contact with a dura mater of the subject.

8. The implantable transducer of claim 1, wherein the housing comprises a lip disposed at a second end of the housing, the lip configured to be mounted to an outer surface of a skull of a subject.

9. The implantable transducer of claim 1, wherein the implantable transducer comprises a cable configured to transmit power or data to or from the implantable transducer.

10. The implantable transducer of claim 1, wherein the sonolucent window comprises a biocompatible polymer, wherein the biocompatible polymer is polymethyl methacrylate (PMMA), or Poly(ether) ether ketone (PEEK), polychlorotrifluoroethylene (PCTFE), polytetrafluoroethylene (PTFE), ultra-high-molecular-weight polyethylene (UHMWPE), polyethylene terephthalate (PET), low density polyethylene (LDPE), polyether block amide (PEBAX), and/or high-density polyethylene (HDPE).

11. The implantable transducer of claim 1, wherein the ultrasound array is fabricated on a complementary metal-oxide semiconductor (CMOS) application specific integrated circuit (ASIC).

12. The implantable transducer of claim 1, wherein the ultrasound array comprises a capacitive micromachined ultrasonic transducer (CMUT), piezoelectric micromachined ultrasonic transducer (PMUT) array or a Lead Zirconate Titanate (PZT) array.

13. The implantable transducer of claim 1, wherein the implantable transducer is configured to couple to one or more wires, the implantable transducer configured to send and further configured to receive data via the one or more wires.

14. The implantable transducer of claim 1, wherein the ultrasound array comprises a plurality of transducer elements, wherein the plurality of transducer elements comprises 100-199, 200-399, 400-999, 1,000-1,499, 1,500-9,999, 10,000-11,999, 12,000-99,000, or 100,000-120,000 transducer elements.

15. The implantable transducer of claim 14, wherein the ultrasound array comprises an n×m matrix, wherein n is in a range of 16-256 transducer elements and m is in a range of 1-256 transducer elements.

16. A system for monitoring or modulating a physiological activity of the subject, comprising:
one or more implantable transducers, an implantable transducer of the one or more implantable transducers corresponding to the implantable transducer of claim 1; and
a controller coupled to each of the one or more implantable transducers, the controller comprising a power source and a processor,
wherein the power source is configured to power each of the one or more implantable transducers, and
wherein the processor is configured to execute a method, the method comprising:
sending one or more signals to the one or more implantable transducers; and
receiving data from the one or more implantable transducers.

17. The system of claim 16, wherein the method further comprises:
emitting, via the one or more implantable transducers, ultrasound waves, wherein the ultrasound waves are configured to modify the physiological activity of the subject.

18. The system of claim 16, wherein the one or more signals are configured to specify the amplitude or the timing of one or more transducer elements of the plurality of transducer elements.

19. The system of claim 16, comprising between one and ten implantable transducers.

20. The system of claim 16, wherein the implantable transducer and the controller are configured to communicate wirelessly.

21. The system of claim 16, wherein the one or more signals are configured to coordinate emission of ultrasound waves via the implantable transducer and further configured to coordinate receipt of the ultrasound waves.

22. The system of claim 20, wherein the controller comprises a clock, and wherein the one or more signals are sent based on predetermined intervals associated with the clock.

23. The system of claim 22, wherein the controller comprises a central clock and the one or more implantable transducers each comprise the clock, and wherein the signals correspond to reset signals associated with the central clock.

24. The system of claim 16, wherein the subject engages in a clinically relevant behavior while the implantable transducer obtains data.

25. The system of claim 24, wherein the clinically relevant behavior comprises activities of daily living, estimates of movement, motion capture, facial expression and response time, self-reported mood, self-reported cognitive state, heart rate, heart rate variability, breathing rate, oxygenation, galvanic skin response, inertial monitoring, or a combination thereof.

26. The system of claim 16, wherein the system is configured to modify the physiological activity of the subject based on the data.

27. The system of claim 26, wherein the modifying the physiological activity of the subject based on the data occurs in real-time.

28. The system of claim 16, wherein the physiological activity of the subject comprises neural activity.

29. A method for monitoring a physiological activity of a subject, the method comprising:
sending, via a controller, one or more signals to one or more implantable transducers,
wherein the controller is located remotely from the one or more implantable transducers and
wherein the one or more implantable transducers are mounted to the skull of the subject; and
receiving data from the one or more implantable transducers.

30. The method of claim 29, wherein the method further comprises emitting, via the one or more implantable transducers, ultrasound waves based on the one or more signals, wherein the ultrasound waves are configured to modify the physiological activity of the subject.
